(19) United States
(12) Patent Application Publication
(10) Pub. No.: US 2026/0224725 A1
(43) Pub. Date: Aug. 6, 2026

(54) POLYMER-OLIGONUCLEOTIDE CONJUGATES

(71) Applicant: PRIMELINK BIOTHERAPEUTICS (SUZHOU) CO., LTD., Suzhou, Jiangsu (CN)

(72) Inventors: Mao YIN, Needham, MA (US); Yingdong LU, Suzhou, Jiangsu (CN); Xiaohe SHI, Suzhou, Jiangsu (CN); Ganyuan XIAO, Suzhou, Jiangsu (CN); Ling XIN, Suzhou, Jiangsu (CN); Ya ZHANG, Suzhou, Jiangsu (CN); Qing ZHANG, Suzhou, Jiangsu (CN); Haitao PAN, Suzhou, Jiangsu (CN)

(21) Appl. No.: 19/152,005

(22) PCT Filed: Jan. 30, 2024
(86) PCT No.: PCT/CN2024/074723

§ 371 Date: Jul. 30, 2025

(30) Foreign Application Priority Data
Jan. 30, 2023 (WO) PCT/CN2023/073869

(51) Int. Cl. A61K 47/68; A61K 47/59; A61K 47/62; C07K 16/28

(52) U.S. Cl. CPC A61K 47/6807; A61K 47/593; A61K 47/62; A61K 47/6849; C07K 16/2839

(57) ABSTRACT

A polymeric scaffold useful for conjugating with a targeting moiety to form a targeting moiety-polymer-oligonucleotide conjugate is described herein. Also disclosed is a targeting moiety-polymer-oligonucleotide conjugate prepared from the scaffold. Compositions comprising the conjugates, methods of their preparation, and methods of treating various disorders with the conjugates or their composition are also described.

### TECHNICAL FIELD

The present disclosure generally relates to compounds, conjugates comprising these compounds, pharmaceutical compositions thereof, and method for the treatment of diseases or disorders with the conjugates or their pharmaceutical compositions.

### BACKGROUND OF THE DISCLOSURE

Traditionally, therapeutic agents are delivered to the body via oral/GI absorption or systemic injection and then to the action site by the blood circulation. However, many challenges still remain to be addressed. For example, many therapeutic agents exhibit limited or otherwise reduced potencies and therapeutic effects because they are either generally subject to partial degradation before they reach a desired target in the body, or accumulate in tissues other than the target, or both.

Bioactive macromolecules, including peptides, proteins and siRNAs, possess many desirable therapeutic features that provide unique opportunities to design precision medicine therapeutics to treat human disease. However, due to their size (>1,000 Da), macromolecules have no bioavailability to cross the cell membrane and enter cells, and therefore, require the use of an efficient delivery agent to access their site of action in the cytoplasm or nucleus.

There is a need to deliver oligonucleotides intact to specifically targeted areas of the body through a system that can stabilize the drug and control the in vivo transfer of the oligonucleotides such that maximum cytotoxicity for the oligonucleotides is achieved.

### SUMMARY OF THE DISCLOSURE

The present disclosure relates to a polymeric scaffold delivery system that exhibits high oligonucleotides load and strong binding to target antigen, thereby efficiently delivering and releasing the oligonucleotides to the target site. The present disclosure also relates to a polymeric scaffold useful to conjugate with a targeting moiety so as to obtain the polymeric scaffold delivery system.

In one aspect, the present disclosure provides a polymeric scaffold of Formula (I) or Formula (II) useful to conjugate with a targeting moiety:

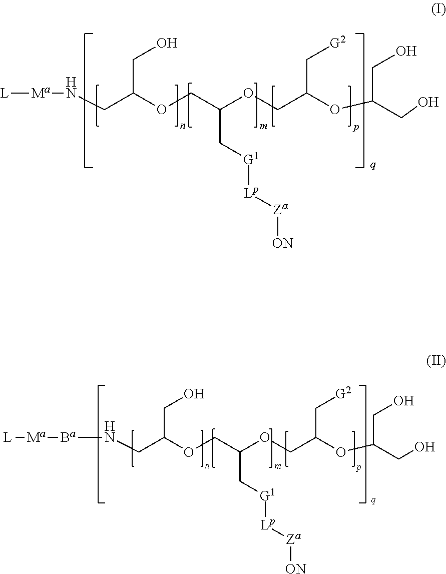

wherein:
the polymeric scaffold comprises linear polyglycerol;
L is a linking moiety comprising a functional group Wp that is capable of forming a covalent bond with the targeting moiety;
M^{a} is a stretcher connecting L to B^{a} or —NH— moiety;
B^{a} is a branching moiety comprising a functional group W_{M} connecting to —NH-moiety;
each G¹ is independently a functional group connecting L^{p} to the linear polyglycerol;
each L^{P} is independently a drug release mechanism between Z^{a} and G¹;
each Z^{a} is independently an endosomal-lysosomal escaping moiety or a conjugation moiety;
each ON is independently an oligonucleotide;
each G² is independently a functional group capable of converting into a charged state;
n is an integer from 0 to 1000;
m is an integer from 0 to 1000;
p is an integer from 0 to 1000; and
q is an integer from 0 to 1000.

In one another aspect, the present disclosure provides a polymeric scaffold of Formula (III) or Formula (IV):

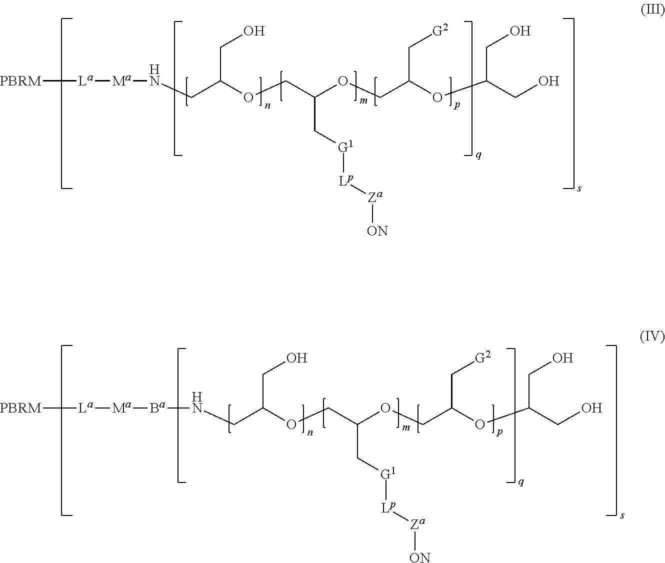

wherein,
the polymeric scaffold comprises linear polyglycerol;
PBRM is a targeting moiety;
each L^{a} is independently a divalent moiety connecting the targeting moiety to M^{a};
each M^{a} is independently a stretcher connecting L^{a} to B^{a} or —NH— moiety;
B^{a} is a branching moiety comprising a functional group W_{M} connecting to —NH-moiety;
each G¹ is independently a functional group connecting L^{p} to the linear polyglycerol;
each L^{P} is independently a drug release mechanism between Z^{a} and G¹;
each Z^{a} is independently an endosomal-lysosomal escaping moiety or a conjugation moiety;
each ON is independently an oligonucleotide;
each G² is independently a functional group capable of converting into a charged state;
n is an integer from 0 to 1000;
m is an integer from 0 to 1000;
p is an integer from 0 to 1000;
q is an integer from 0 to 1000; and
s is an integer from 1 to 8.

In a further aspect, the present disclosure provides a pharmaceutical composition comprising a polymeric scaffold or conjugate described herein and a pharmaceutically acceptable carrier.

In another aspect, the present disclosure provides a method of treating diseases in a subject in need thereof, comprising administering to the subject a therapeutic effective amount of the polymeric scaffold or conjugate described herein, or the pharmaceutical composition provided herein.

### DETAILED DESCRIPTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination.

It must be noted that, as used in the specification and the appended claims, the singular forms “a,” “an,” and “the” include plural forms of the same unless the context clearly dictates otherwise. In this specification and in the claims that follow, reference will be made to a number of terms that shall be defined to have the following meanings unless a contrary intention is apparent.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

At various places in the present disclosure, linking substituents are described. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When any variable (e.g., R) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, a dash “-” at the front or end of a chemical group is used, a matter of convenience, to indicate a point of attachment for a substituent. For example, —OH is attached through the carbon atom; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named. As used herein, a solid line coming out of the center of a ring indicates that the point of attachment for a substituent on the ring can be at any ring atom. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

The term “about”, when used in connection with a numerical value, means that a collection or range of values is included. For example, “about X” includes a range of values that are ±20%, ±10%, ±5%, ±2%, ±1%, 0.5%, ±0.2%, or ±0.1% of X, where X is a numerical value. In one embodiment, the term “about” refers to a range of values which are 5% more or less than the specified value. In another embodiment, the term “about” refers to a range of values which are 2% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the expressions “n is an integer between 1 and 6” and “n being an integer of 1 to 6” both mean “x being 1, 2, 3, 4, 5, or 6”.

As used herein, the term “Cᵢ₋ⱼ” indicates a range of the carbon atoms numbers, wherein i and j are integers and the range of the carbon atoms numbers includes the endpoints (i.e., i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms. In similar manner, the term “m-n membered” ring, wherein m and n are integers and n is greater than m, refers to a ring containing m to n atoms.

As used herein, the term “aliphatic” includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, alkynyl moieties.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to a linear or branched-chain alkyl having i to j carbon atoms. For example, alkyl groups contain 1 to 10, 1 to 9, 1 to 8, 1 to 7, 1 to 6, 1 to 5, 1 to 4, 1 to 3, or 1 to 2 carbon atoms. Examples of “C₁₋₆ alkyl” include, but are not limited to, methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 2-ethyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. Examples of alkenyl group include, but are not limited to, ethylenyl (or vinyl), propenyl (allyl), butenyl, pentenyl, 1-methyl-2 buten-1-yl, 5-hexenyl, and the like.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkenyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like.

As used herein, the term “amino” refers to the group —NR^{a}R^{b}, wherein R^{a} and R^{b} are independently selected from groups consisting of hydrogen, alkyl, haloalkyl, aryl, heteroaryl, cycloalkyl, and heterocyclyl and each of which may be optionally substituted.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused or bridged. Examples of polycyclic aryl include, but are not limited to, benzofuranyl, indanyl, phthalimidyl, naphthimidyl, phenanthridinyl, or tetrahydronaphthyl, and the like.

As used herein, the term “cycloalkyl”, whether as part of another term or used independently, refers to a monovalent non-aromatic, saturated or partially unsaturated monocyclic and polycyclic ring system, in which all the ring atoms are carbon and which contains at least three ring forming carbon atoms. In some embodiments, the cycloalkyl group may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. The cycloalkyl group may be saturated or partially unsaturated. In some embodiments, the cycloalkyl group may be a saturated cyclic alkyl group. In some embodiments, the cycloalkyl group may be a partially unsaturated cyclic alkyl group that contains at least one double bond or triple bond in its ring system.

In some embodiments, the cycloalkyl group may be saturated or partially unsaturated monocyclic carbocyclic ring system, examples of which include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl.

In some embodiments, the cycloalkyl group may be saturated or partially unsaturated polycyclic (e.g., bicyclic and tricyclic) carbocyclic ring system, which can be arranged as a fused, spiro or bridged ring system. As used herein, the term “fused ring” refers to a ring system having two rings sharing two adjacent atoms, the term “spiro ring” refers to a ring systems having two rings connected through one single common atom, and the term “bridged ring” refers to a ring system with two rings sharing three or more atoms. Examples of fused carbocyclyl include, but are not limited to, naphthyl, benzopyrenyl, anthracenyl, acenaphthenyl, fluorenyl and the like. Examples of spiro carbocyclyl include, but are not limited to, spiro[5.5]undecanyl, spiro-pentadienyl, spiro[3.6]-decanyl, and the like. Examples of bridged carbocyclyl include, but are not limited to bicyclo[1,1,1]pentenyl, bicyclo[2,2,1]heptenyl, bicyclo[2.2.1]heptanyl, bicyclo[2.2.2]octanyl, bicyclo[3.3.1]nonanyl, bicyclo[3.3.3]undecanyl, and the like.

As used herein, the term “halo” or “halogen” refers to an atom selected from fluorine (or fluoro), chlorine (or chloro), bromine (or bromo) and iodine (or iodo).

As used herein, the term “heteroatom” refers to nitrogen, oxygen, sulfur or phosphorus, and includes any oxidized form of nitrogen, sulfur or phosphorus, and any quaternized form of a basic nitrogen.

As used herein, the term “heteroaliphatic” refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be branched or linear unbranched. As will be appreciated by one of ordinary skill in the art, “heteroaliphatic” is intended herein to include, but is not limited to, heteroalkyl, heteroalkenyl, heteroalkynyl moieties. In certain embodiments, heteroaliphatic moieties are substituted (“substituted heteroaliphatic”) by independent replacement of one or more of the hydrogen atoms thereon with one or more moieties including, but not limited to aliphatic; heteroaliphatic; cycloalkyl; heterocycloalkyl; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; - or -GR^{G1}, wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}_, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})_-NR^{G2}C(═NR^{G3})—, —NR^{G2}SO2-, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, cycloalkyl, heterocycloalkyl, aryl, heteroaryl, alkylaryl, or alkylheteroaryl moiety. Additional examples of generally applicable substituents are illustrated by the specific embodiments shown in the Examples that are described herein.

As used herein, the term “heteroalkyl” refers to an alkyl, at least one of the carbon atoms of which is replaced with a heteroatom selected from N, O, or S. The heteroalkyl may be a carbon radical or heteroatom radical (i.e., the heteroatom may appear in the middle or at the end of the radical), and may be optionally substituted independently with one or more substituents described herein. The term “heteroalkyl” encompasses alkoxyl and heteroalkoxy radicals.

As used herein, the term “heteroalkenyl” refers to an alkenyl, at least one of the carbon atoms of which is replaced with a heteroatom selected from N, O, or S. The heteroalkenyl may be a carbon radical or heteroatom radical (i.e., the heteroatom may appear in the middle or at the end of the radical), and may be optionally substituted independently with one or more substituents described herein.

As used herein, the term “heteroalkynyl” refers to an alkynyl, at least one of the carbon atoms of which is replaced with a heteroatom selected from N, O, or S. The heteroalkynyl may be a carbon radical or heteroatom radical (i.e., the heteroatom may appear in the middle or at the end of the radical), and may be optionally substituted independently with one or more substituents described herein.

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, heteroaryl, cycloaliphatic, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocycloalkyl” refers to a saturated or partially unsaturated cycloalkyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocycloalkyl is a saturated heterocycloalkyl. In some embodiments, the heterocycloalkyl is a partially unsaturated heterocycloalkyl having one or more double bonds in its ring system. In some embodiments, the heterocycloalkyl may contains any oxidized form of carbon, nitrogen or sulfur, and any quaternized form of a basic nitrogen. The heterocycloalkyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

Heterocycloalkyl group may be monocyclic. Examples of monocyclic heterocycloalkyl include, but are not limited to oxetanyl, 1,1-dioxothietanylpyrrolidyl, tetrahydrofuryl, tetrahydropyranyl, tetrahydrothienyl, azetidinyl, pyrrolyl, furanyl, thienyl, pyrazolyl, imidazolyl, triazolyl, oxazolyl, thiazolyl, piperidyl, piperazinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like.

Heterocycloalkyl group may be polycyclic, including the fused, spiro and bridged ring systems. The fused heterocycloalkyl group includes radicals wherein the heterocycloalkyl radicals are fused with a saturated, partially unsaturated, or fully unsaturated (i.e., aromatic) carbocyclic or heterocyclic ring. Examples of fused heterocycloalkyl include, but are not limited to, phenyl fused ring or pyridinyl fused ring, such as quinolinyl, isoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, imidazo[1,2-a]pyridinyl, furo[3,4-d]pyrimidinyl, pyrrolo[3,4-d]pyrimidinyl, dihydrofuro[3,4-b]pyridinyl groups, and the like. Examples of spiro heterocycloalkyl include, but are not limited to, spiropyranyl, spirooxazinyl, 5-aza-spiro[2.4]heptanyl, 6-aza-spiro[2.5]octanyl, 6-aza-spiro[3.4]octanyl, 2-oxa-6-aza-spiro[3.3]heptanyl, 2-oxa-6-aza-spiro[3.4]octanyl, 6-aza-spiro[3.5]nonanyl, 7-aza-spiro[3.5]nonanyl, 1-oxa-7-aza-spiro[3.5]nonanyl, 3,8-dioxa-1-azaspiro[4.5]dec-1-enyl and the like. Examples of bridged heterocycloalkyl include, but are not limited to, 3-aza-bicyclo[3.1.0]hexanyl, 8-aza-bicyclo[3.2.1]octanyl, 1-aza-bicyclo[2.2.2]octanyl, 2-aza-bicyclo[2.2.1]heptanyl, 1,4-diazabicyclo[2.2.2]octanyl, and the like.

As used herein, the term “hydroxyl” refers to —OH.

As used herein, the term “leaving group” refers to a molecular fragment that departs with a pair of electrons in heterolytic bond cleavage. Leaving groups can be anions or neutral molecules. Leaving groups include, but are not limited to halides such as Cl⁻, Br⁻, and I⁻, sulfonate esters, such as para-toluenesulfonate (“tosylate”, TsO⁻), and RC(O)O⁻ in which R is hydrogen, aliphatic, heteroaliphatic, cycloalkyl, or heterocycloalkyl moiety.

As used herein, the term “optional” or “optionally” means that the subsequently described event or circumstance may or may not occur, and that the description includes instances where the said event or circumstance occurs and instances in which it does not.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “protecting group” means that a particular functional moiety, e.g., O, S, or N, is temporarily blocked so that a reaction can be carried out selectively at another reactive site in a multifunctional compound. In some embodiments, a protecting group reacts selectively in good yield to give a protected substrate that is stable to the projected reactions. The protecting group must be selectively removed in good yield by readily available, preferably nontoxic reagents that do not attack the other functional groups. The protecting group forms an easily separable derivative (more preferably without the generation of new stereogenic centers). The protecting group has a minimum of additional functionality to avoid further sites of reaction. As detailed herein, oxygen, sulfur, nitrogen and carbon protecting groups may be utilized. For example, in some embodiments, certain exemplary oxygen protecting groups may be utilized. These oxygen protecting groups include, but are not limited to methyl ethers, substituted methyl ethers (e.g., MOM (methoxymethyl ether), MTM (methylthiomethyl ether), BOM (benzyloxymethyl ether), and PMBM (p-methoxybenzyloxymethyl ether)), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, and TBDPS (t-butyldiphenyl silyl ether), esters (e.g., formate, acetate, benzoate (Bz), trifluoroacetate, and dichloroacetate), carbonates, cyclic acetals and ketals. In some other embodiments, nitrogen protecting groups are utilized. Nitrogen protecting groups, as well as protection and deprotection methods are known in the art. Nitrogen protecting groups include, but are not limited to, carbamates (including methyl, ethyl and substituted ethyl carbamates (e.g., Troc), amides, cyclic imide derivatives, N-Alkyl and N-Aryl amines, imine derivatives, and enamine derivatives. In yet other embodiments, certain exemplary sulphur protecting groups may be utilized. The sulfur protecting groups include, but are not limited to those oxygen protecting group describe above as well as aliphatic carboxylic acid (e.g., acrylic acid), maleimide, vinyl sulfonyl, and optionally substituted maleic acid. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

As used herein, the term “leaving group” refers to a molecular fragment that departs with a pair of electrons in heterolytic bond cleavage. Leaving groups can be anions or neutral molecules. Leaving groups include, but are not limited to halides such as Cl⁻, Br⁻, and I⁻, sulfonate esters, such as para-toluenesulfonate (“tosylate”, TsO⁻), and RC(O)O⁻ in which R is hydrogen, an aliphatic, heteroaliphatic, carbocyclic, or heterocycloalkyl moiety.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. The substituents may include, but not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

As used herein, the term “oligonucleotide” or “polynucleotide” refers to deoxyribonucleic acid (DNA) and ribonucleic acid (RNA) and combinations of DNA, RNA and other natural and synthetic nucleotides, including protein nucleic acid (PNA). DNA may be in form of cDNA, in vitro polymerized DNA, plasmid DNA, parts of a plasmid DNA, genetic material derived from a virus, linear DNA, vectors (P1, PAC, BAC, YAC, and artificial chromosomes), expression vectors, expression cassettes, chimeric sequences, recombinant DNA, chromosomal DNA, anti-sense DNA, or derivatives of these groups. RNA may be in the form of messenger RNA (mRNA), in vitro polymerized RNA, recombinant RNA, transfer RNA (tRNA), small nuclear RNA (snRNA), ribosomal RNA (rRNA), chimeric sequences, anti-sense RNA, interfering RNA, small interfering RNA (siRNA), microRNA (miRNA), ribozymes, external guide sequences, small non-messenger RNAs (snmRNA), untranslatedRNA (utRNA), snoRNAs (24-mers, modified snmRNA that act by an anti-sense mechanism), tiny non-coding RNAs (tncRNAs), small hairpin RNA (shRNA), or derivatives of these groups. In addition, DNA and RNA may be single, double, triple, or quadruple stranded. Double, triple, and quadruple stranded polynucleotide may contain both RNA and DNA or other combinations of natural and/or synthetic nucleic acids. Oligonucleotides can be chemically modified. The use of chemically modified oligonucleotides can improve various properties of the oligonucleotides including, but not limited to resistance to nuclease degradation in vivo, cellular uptake, activity, and sequence-specific hybridization. Chemical modifications can include modifications of all or some of the sugar groups, base groups, phosphate groups of the nucleic acid and 3′ and 5′ ends of the oligonucleotide. Non-limiting examples of such chemical modifications include: phosphorothioate internucleotide linkages, LNA, 2′-O-methyl ribonucleotides, 2′-deoxy-2′-fluoro ribonucleotides, 2′-deoxy ribonucleotides, “universal base” nucleotides, 5-C-methyl nucleotides, and inverted deoxyabasic residue incorporation. These chemical modifications, when used in various oligonucleotide constructs, are shown to preserve oligonucleotide activity in cells while at the same time, dramatically increasing the serum stability of these compounds. Chemically modified siRNA can also minimize the possibility of activating interferon activity in humans. An oligonucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. An oligonucleotide may be further modified after polymerization, such as by conjugation with a labeling component. The oligonucleotide preferably comprises from 8 to 30 nucleotide building blocks, more preferably 12 to 28 nucleotide building blocks, even more preferred 16 to 26 nucleotide building blocks, most preferred 18, 20 or 22 nucleotide building blocks. As used herein, the term “4′-O-methoxy” (also 4′-OMe and 4′-OCH₃) refers to an O-methoxy modification of the 4′ position of a furanose ring. As used herein, the term “2′-F” refers to an F modification of the 2′ position of a furanose ring. As used herein, the term “glycol nucleic acid (“GNA”)” refers to a nucleic acid similar to DNA or RNA but differing in the composition of its sugar-phosphodiester backbone, using propylene glycol in place of ribose or deoxyribose.

As used herein, the term “transforming growth factor (TGFβ)” refers to a disulfide linked dimer that is synthesized as a preproprotein of about 400 amino acids (aa) which is cleaved prior to secretion to produce mature TGFβ. The N-terminal cleavage fragment, known as the “latency-associated peptide” (LAP), may remain noncovalently bound to the dimer, thereby inactivating TGFβ. TGFβ isolated in vivo, is found predominantly in the inactive, “latent” form, i.e., associated with LAP. Latent TGFβ complex may be activated in several ways, for example, by binding to a cell surface receptor called the cation-independent mannose-6-phosphate/insulin-like growth factor II receptor. Binding occurs through mannose-6-phosphate residues attached at glycosylation sites within LAP. Upon binding to the receptor, TGFβ is released in its mature form. Mature, active TGFβ is then free to bind to its receptor and exert its biological functions. The major TGFβ binding domain in the type II TGFβ receptor has been mapped to a 19 amino acid sequence (Demetriou et al., J. Biol. Chem., 271:12755, 1996). See also U.S. Pat. No. 7,867,496. Currently, there are five known isoforms of TGFβ (TGFβ1 to TGFβ5; TGFβ1-3 are mammalian, TGFβ4 is found in chicken; and TGFβ5 found in frog), all of which are homologous among each other (60-80% identity), form homodimers of about 25 kDa, and act upon common TGFβ receptors (TGFβ-RI, TGFβ-RII, TGFβ-RIIB, and TGFβ-RIII). The structural and functional aspects of TGFβ as well as TGFβ receptors are well-known in the art (see, for example, Cytokine Reference, eds. Oppenheim et al., Academic Press, San Diego, Calif., 2001). TGFβ is well-conserved among species. For example, the amino acid sequences of rat and human mature TGFβ1s are nearly identical. See also U.S. Pat. No. 7,867,496.

TGFβ is one of the most potent regulators of the production and deposition of extracellular matrix. It stimulates the production and affects the adhesive properties of the extracellular matrix by two major mechanisms. First, TGFβ stimulates fibroblasts and other cells to produce extracellular-matrix proteins and cell-adhesion proteins, including collagen, fibronectin, and integrins. Second, TGFβ decreases the production of enzymes that degrade the extracellular matrix, including collagenase, heparinase, and stromelysin, and increases the production of proteins that inhibit enzymes that degrade the extracellular matrix, including plasminogen-activator inhibitor type 1 and tissue inhibitor of metalloprotease. The net effect of these changes is to increase the production of extracellular-matrix proteins and either to increase or to decrease the adhesive properties of cells in a cell-specific manner (see Blobe G C et al., May 2000, “Role of transforming growth factor beta in human disease”, N. Engl. J. Med. 342 (18), 1350-1358). Targeting TGFβ has been proposed as a potential therapeutic measure for example in glaucoma. Concerning various aspects of TGFβ in the pathogenesis of glaucoma, therapies should be directed to modulate its production, activation, interaction with receptors, downstream intracellular regulatory mechanisms and/or the final structural and ECM changes (see Prendes M A et al., Br J Ophthalmol (2013), 97, 680-686).

As used herein, the term “endosomal escape moiety”, “lysosomal escape moiety” or “endosomal-lysosomal escape moiety”, refers to a moiety which enhances the release of endosomal or lysosomal contents or allows for the escape of a molecule from an internal cellular compartment such as an endosome or lysosome. An endosomal escape moiety or a lysosomal escape moiety generally destabilizes an endosomal or lysosomal membrane. In some embodiments, the endosomal escape moiety is a hydrophobic domain or a cationic domain. Cationic domains include protein transduction domains (PTDs), guanidinium groups, primary amines, secondary amines, tertiary amines, complex amino groups, and ionizable amines. In some embodiments, a cationic domain (cationic charge domain) can comprise multiple cationic charges (e.g., 1-10, 11-20, 21-50 or more) on a single unit structure. In some embodiments, the endosomal escape moiety is endosomal escape domain (EED). In some embodiments, the endosomal escape moiety is a cationic polymer.

As used herein, the term “cell penetrating peptides” (“CPPs”) is generally used to designate short peptides that are able to transport different types of cargo molecules across plasma membrane, and, thus, facilitate cellular uptake of various molecular cargoes (from nanosize particles to small chemical molecules and large fragments of DNA). “Cellular internalization” of the cargo molecule linked to the cell penetrating peptide generally means transport of the cargo molecule across the plasma membrane and thus entry of the cargo molecule into the cell. Depending on the particular case, the cargo molecule can, then, be released in the cytoplasm, directed to an intracellular organelle, or further presented at the cell surface. Cell penetrating ability, or internalization, of the cell penetrating peptide or complex comprising the cell penetrating peptide, according to the present disclosure can be checked by standard methods known to one skilled in the art, including flow cytometry or fluorescence microscopy of live and fixed cells, immunocytochemistry of cells transduced with said peptide or complex, and Western blot. In some embodiments, CPP is selected from Tat, Penetratin, Pep-1, MPG, Polyarginine (R9, R8), R6/W3, SAP, SAP(E), CyLop-1, gH 625, GALA, TP10, CADY, L17E, MPPs, Ac-1, Ent Ac-1, Ac-2, Peptide 3, RR5-App, RR4-App, RR3-aPP, TATp-D, R4-R4, R5-R5, [WR]₄, Cyclic Tat, cFΦR4, Danamide D, Pro-(Xaa)₄-Tyr, Cyclic sC18, RRRRΦF, BIM SAHB9, SAH-SOS1, 4-R, 4-W, and Sp-CC-PEG₂₀₀₀.

As used herein, the term “endosomal escape domain (EED)” refers to a domain of 1-8 amino acids comprising from 2-6 aromatic groups (e.g., tryptophan has 2 aromatic groups) and a spacer of from 2-18 polyethylene glycol (PEG) moieties. In some embodiments, the EED comprises 4 aromatic groups. In some embodiments, the EED does not comprise more than 3 phenylalanines in series. In some embodiments, the EED comprises amino acids having aromatic rings that are spaced from one another by at least one non-aromatic containing amino acids. In some embodiments, the EED comprises a peptide selected from the group consisting of GG, GFFG, GFWFG, GWGGWG, GWG, GFWG, GWWG, GWWG, GWG, GFWG, and GWWG or unnatural amino acids having structure that correspond to G, W, or F. In some embodiments, the EED comprises from 1-18 PEG moieties. In some embodiments, the EED comprises 3-8 PEG moieties. In some embodiments, the EED comprises 6 PEG moieties.

As used herein, the term “cationic polymer” means a polymer having a net cationic charge. Furthermore, it is understood that the cationic polymers described herein are typically synthesized according to known methods from polymer-forming monomers (e.g., (meth)acrylamide monomers, DADMAS monomers, etc.). As used herein, the resulting polymer is considered the “polymerized portion” of the cationic polymer. However, after the synthesis reaction is complete, a portion of the polymer-forming monomers may remain unreacted and/or may form oligomers. As used herein, the unreacted monomers and oligomers are considered the “unpolymerized portion” of the cationic polymer. As used herein, the term “cationic polymer” includes both the polymerized portion and the unpolymerized portion unless stated otherwise. In some embodiments, the cationic polymer comprises an unpolymerized portion of the cationic polymer. In some embodiments, the cationic polymer comprises less than about 50%, or less than about 35%, or less than about 20%, or less than about 15%, or less than about 10%, or less than about 5%, or less than about 2%, by weight of the cationic polymer, of an unpolymerized portion. The unpolymerized portion may comprise polymer-forming monomers, cationic polymer-forming monomers, or DADMAC monomers, and/or oligomers thereof. In some embodiments, the cationic polymer comprises more than about 50%, or more than about 65%, or more than about 80%, or more than about 85%, or more than about 90%, or more than about 95%, or more than about 98%, by weight of the cationic polymer, of a polymerized portion. Furthermore, it is understood that the polymer-forming monomers, once polymerized, may be modified to form polymerized repeat/structural units. For example, polymerized vinyl acetate may be hydrolyzed to form vinyl alcohol.

As used herein, the term “conjugation moiety” refers to a moiety conjugating the oligonucleotide ON to the parent molecule. In some embodiments, the conjugation moiety conjugates the oligonucleotide ON to the drug release mechanism L^{p}. In some embodiments, the conjugation moiety is **—NH—(CH₂)ₙ₇—*, **—S—(CH₂)ₙ₇—*, or **—O—(CH₂)ₙ₇—*, wherein n7 is an integer from 1 to 6, * is the site covalently attached to ON, and ** is the site covalently attached to L^{p}.

As used herein, the term “targeting moiety” refers to a molecule that recognizes and binds to a cell surface marker or receptor such as, a transmembrane protein, surface immobilized protein, or protoglycan. Examples of targeting moiety include but are not limited to, antibodies or fragment thereof, lipocalins, proteins, peptides or peptide mimics, and the like. The targeting moiety, in addition to targeting the polymeric scaffold to a specific cell, tissue or location, may also have certain therapeutic effect such as antiproliferative (cytostatic and/or cytotoxic) activity against a target cell or pathway. The targeting moiety comprises or may be engineered to comprise at least one chemically reactive group such as, —COOR, —SH, amine or a chemically reactive amino acid moiety or side chains such as, for example, tyrosine, histidine, cysteine, or lysine. In some embodiments, a targeting moiety may be a ligand which specifically binds or complexes with a cell surface molecule, such as a cell surface receptor or antigen, for a given target cell population. Following specific binding or complexing of the ligand with its receptor, the cell is permissive for uptake of the ligand or ligand-oligonucleotide-conjugate, which is then internalized into the cell. As used herein, a ligand that “specifically binds or complexes with” or “targets” a cell surface molecule preferentially associates with a cell surface molecule via intermolecular forces.

As used herein, the term “ligand” refers to a variety of chemical or biological molecules, which can have a specific binding affinity to a selected target, wherein the selected target can be, for example, a cell surface receptor, a cell surface antigen, a cell, a tissue, an organ, etc. In some embodiments, the ligand can specifically bind to a protein or a marker expressed on the surface of a target cell. In some embodiments, the ligand of the present disclosure binds to a cell surface protein or marker with an affinity of 10⁻⁶-10⁻¹¹ M (K_{d} value). In some embodiments, the ligand of the present disclosure binds to a cell surface protein or marker with an affinity of at least 10⁻⁷, at least 10⁻⁸ and at least 10⁻⁹ M (K_{d} value). In some embodiments, the ligand of the present disclosure binds to a cell surface protein or marker with an affinity of less than 10⁻⁶, less than 10⁻⁷ and less than 10⁻⁸ M (K_{d} value). In some embodiments, the ligand of the present disclosure binds to a cell surface protein or marker with a certain affinity, wherein the certain affinity refers to the affinity of the ligand to a target cell surface protein or marker which is at least two, three, four, five, six, eight, ten, twenty, fifty, one hundred or more times higher than that to a non-target cell surface protein or marker. In some embodiments, the expression of the cell surface protein or marker of the present disclosure in target cells (e.g. cancer cells) is significantly higher than that in normal cells. The term “significantly” as used herein refers to statistically significant differences, or significant differences that can be recognized by a person skilled in the art.

As used herein, the term “antibody” includes any immunoglobulin, monoclonal antibody, polyclonal antibody, multivalent antibody, multispecific antibody, or bispecific (bivalent) antibody or a functional portion thereof that binds to a specific antigen. A native intact antibody comprises two heavy chains (H) and two light (L) chains inter-connected by disulfide bonds. Each heavy chain consists of a variable region (VH) and a first, second, and third constant region (CH1, CH2 and CH3, respectively), while each light chain consists of a variable region (VL) and a constant region (CL). Mammalian heavy chains are classified as α, δ, ε, γ, and μ, and mammalian light chains are classified as λ or κ. The variable regions of the light and heavy chains are responsible for antigen binding. The variables region in both chains are generally subdivided into three regions of hypervariability called the complementarity determining regions (CDRs) (light (L) chain CDRs including LCDR1, LCDR2, and LCDR3, heavy (H) chain CDRs including HCDR1, HCDR2, HCDR3). CDR boundaries for the antibodies and antigen-binding fragments disclosed herein may be defined or identified by the conventions of Kabat, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. Therefore, each VH and VL comprises of three CDRs and four FRs in the following order (amino acid residues N terminus to C terminus): FR1, CDR1, FR2, CDR2, FR3, CDR3, FR4. The constant regions of the heavy and light chains are not involved in antigen binding, but exhibit various effector functions. Antibodies are assigned to the five major classes based on the amino acid sequence of the constant region of their heavy chain: IgA, IgD, IgE, IgG, and IgM, which are characterized by the presence of α, δ, ε, γ, and μ heavy chains, respectively. Subclasses of several of the major antibody classes are such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

As used herein, the term “Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

As used herein, the term “Fab′” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

As used herein, the term “F(ab′)₂” refers to a dimer of Fab′ that comprises two light chains and part of two heavy chains.

As used herein, the term “Fc” with regard to an antibody refers to that portion of the antibody consisting of the second and third constant regions of a first heavy chain bound to the second and third constant regions of a second heavy chain via disulfide bond. IgG and IgM Fc regions contain three heavy chain constant regions (second, third and fourth heavy chain constant regions in each chain). It can be obtained by papain digestion of an antibody. The Fc portion of the antibody is responsible for various effector functions such as ADCC, and CDC, but does not function in antigen binding.

As used herein, the term “Fv” with regard to an antibody refers to the smallest fragment of the antibody to bear the complete antigen binding site. A Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

As used herein, the term “single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)). A “scFv dimer” refers to a single chain comprising two heavy chain variable regions and two light chain variable regions with a linker. An “scFv dimer” may be a bivalent diabody or bivalent ScFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or eptipoes) or different antigens (or eptipoes). A “scFv dimer” may also be a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

As used herein, the term “single-chain Fv-Fc antibody” or “scFv-Fc” refers to an engineered antibody consisting of a scFv connected to the Fc region of an antibody.

As used herein, the term “camelized single domain antibody,” “heavy chain antibody,” “nanobody” or “HCAb” refers to an antibody that contains two VH domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)). “Diabodies” include small antibody fragments with two antigen-binding sites, wherein the fragments comprise a V_{H} domain connected to a V_{L} domain in a single polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). The two domains on the same chain cannot be paired, because the linker is too short, thus, the domains are forced to pair with the complementary domains of another chain, thereby creating two antigen-binding sites. The antigen-binding sites may target the same of different antigens (or epitopes).

As used herein, the term “domain antibody” refers to an antibody fragment containing only the variable region of a heavy chain or the variable region of a light chain. In some embodiments, two or more V_{H} domains are covalently joined with a peptide linker to form a bivalent or multivalent domain antibody. The two V_{H} domains of a bivalent domain antibody may target the same or different antigens.

As used herein, the term “(dsFv)₂” refers to an antigen binding fragment consisting of three peptide chains: two V_{H} moieties linked by a peptide linker and bound by disulfide bridges to two V_{L} moieties.

As used herein, the term “bispecific ds diabody” refers to an antigen binding fragment consisting of V_{H1}-V_{L2} (linked by a peptide linker) bound to V_{L1}-V_{H2} (also linked by a peptide linker) via a disulfide bridge between V_{H1} and V_{L1}.

As used herein, the term “bispecific dsFv” or “dsFv-dsFv” refers to a antigen binding fragment consisting of three peptide chains: a V_{H1}-V_{H2} moiety wherein the heavy chains are bound by a peptide linker (e.g., a long flexible linker) and paired via disulfide bridges to V_{L1} and V_{L2} moieties, respectively. Each disulfide paired heavy and light chain has a different antigen specificity.

In some embodiment, the antibody or its antigen binding fragment is chimeric or humanized.

As used herein, the term “chimeric” refers to an antibody or antigen-binding fragment that has a portion of heavy and/or light chain derived from one species, and the rest of the heavy and/or light chain derived from a different species. In an illustrative example, a chimeric antibody may comprise a constant region derived from human and a variable region derived from a non-human species, such as from mouse.

As used herein, the term “humanized”, with reference to antibody or antigen-binding fragment, refers to the antibody or the antigen-binding fragment comprises CDRs derived from non-human animals (e.g., a rodent, rabbit, dog, goat, horse, or chicken), FR regions derived from human, and when applicable, the constant regions derived from human. In some embodiments, the constant regions from a human antibody are fused to the non-human variable regions. A humanized antibody or antigen-binding fragment is useful as human therapeutics. In some embodiments, the non-human animal is a mammal, for example, a mouse, a rat, a rabbit, a goat, a sheep, a guinea pig, a hamster, or a non-human primate (for example, a monkey (e.g., cynomolgus or rhesus monkey) or an ape (e.g., chimpanzee, gorilla, simian or affen)). In some embodiments, the humanized antibody or antigen-binding fragment is composed of substantially all human sequences except for the CDR sequences which are non-human. In some embodiments, the humanized antibody or antigen-binding fragment is modified to improve the antibody performance, such as binding or binding affinity. For example, one or more amino acid residues in one or more non-human CDRs are altered to reduce potential immunogenicity in human, wherein the altered amino acid residues either are not critical for immunospecific binding or the alterations are conservative changes, such that the binding of the humanized antibody to the antigen is not significantly affected. In some embodiments, the FR regions derived from human may comprise the same amino acid sequence as the human antibody from which it is derived, or it may comprise some amino acid changes, for example, no more than 10, 9, 8, 7, 6, 5, 4, 3, 2, or 1 changes of amino acid. In some embodiments, such change in amino acid could be present in heavy chain FR regions only, in light chain FR regions only, or in both chains.

As used herein, the term “natural amino acid” refers to any one of the common, naturally occurring L-amino acids found in naturally occurring proteins: glycine (Gly), alanine (Ala), valine (Val), leucine (Leu), isoleucine (Ile), lysine (Lys), arginine (Arg), histidine (His), proline (Pro), serine (Ser), threonine (Thr), phenylalanine (Phe), tyrosine (Tyr), tryptophan (Trp), aspartic acid (Asp), glutamic acid (Glu), asparagine (Asn), glutamine (Gln), cysteine (Cys) and methionine (Met).

As used herein, the term “non-natural amino acid” as used herein refers to any amino acid which is not a natural amino acid. This includes, for example, amino acids that comprise α-, β-, ω-, D-, L-amino acyl residues. More generally, the non-natural amino acid comprises a residue of the general formula

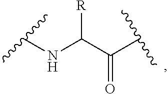

wherein the side chain R is other than the amino acid side chains occurring in nature. Exemplary unnatural amino acids, include, but are not limited to, sarcosine (N-methylglycine), citrulline (cit), homocitrulline, β-ureidoalanine, thiocitrulline, hydroxyproline, allothreonine, pipecolic acid (homoproline), α-aminoisobutyric acid, tert-butylglycine, tert-butylalanine, allo-isoleucine, norleucine, α-methylleucine, cyclohexylglycine, β-cyclohexylalanine, β-cyclopentylalanine, α-methylproline, phenylglycine, α-methylphenylalanine and homophenylalanine.

As used herein, the term “polypeptide”, “protein” or “peptide” can be a single amino acid or a polymer of amino acids. The polypeptide, protein or peptide as described in the present disclosure may contain naturally-occurring amino acids and non-naturally-occurring amino acids, or analogs and mimetics thereof. The polypeptide, protein or peptide can be obtained by any method well known in the art, for example, but not limited to, by an isolation and a purification from natural materials, a recombinant expression, a chemical synthesis, etc.

As used herein, the term “biocompatible” as used herein is intended to describe compounds that exert minimal destructive or host response effects while in contact with body fluids or living cells or tissues. Thus, a biocompatible group, as used herein, refers to an aliphatic, cycloalkyl, heteroaliphatic, heterocycloalkyl, aryl, or heteroaryl moiety, which falls within the definition of the term biocompatible, as defined above and herein. The term “biocompatibility” as used herein, is also taken to mean that the compounds exhibit minimal interactions with recognition proteins, e.g., naturally occurring antibodies, cell proteins, cells and other components of biological systems, unless such interactions are specifically desirable. Thus, substances and functional groups specifically intended to cause the above minimal interactions, e.g., drugs and prodrugs, are considered to be biocompatible. In some embodiments, compounds are “biocompatible” if their addition to normal cells in vitro, at concentrations similar to the intended systemic in vivo concentrations, results in less than or equal to 1% cell death during the time equivalent to the half-life of the compound in vivo (e.g., the period of time required for 50% of the compound administered in vivo to be eliminated/cleared), and their administration in vivo induces minimal and medically acceptable inflammation, foreign body reaction, immunotoxicity, chemical toxicity and/or other such adverse effects. As used herein, the term “normal cells” refers to cells that are not intended to be destroyed or otherwise significantly affected by the compound being tested.

As used herein, “biodegradable” polymers are polymers that are susceptible to biological processing in vivo. As used herein, “biodegradable” compounds or moieties are those that, when taken up by cells, can be broken down by the lysosomal or other chemical machinery or by hydrolysis into components that the cells can either reuse or dispose of without significant toxic effect on the cells. The term “biocleavable” as used herein has the same meaning of “biodegradable”. The degradation fragments preferably induce little or no organ or cell overload or pathological processes caused by such overload or other adverse effects in vivo. Examples of biodegradation processes include enzymatic and non-enzymatic hydrolysis, oxidation and reduction. Suitable conditions for non-enzymatic hydrolysis of the biodegradable protein-polymer-oligonucleotide conjugates (or their components, e.g., the biodegradable polymeric carrier and the linkers between the carrier and the antibody or the oligonucleotide molecule) described herein, for example, include exposure of the biodegradable conjugates to water at a temperature and a pH of lysosomal intracellular compartment. Biodegradation of some protein-polymer-oligonucleotide conjugates (or their components, e.g., the biodegradable polymeric carrier and the linkers between the carrier and the antibody or the oligonucleotide molecule), can also be enhanced extracellularly, e.g. in low pH regions of the animal body, e.g. an inflamed area, in the close vicinity of activated macrophages or other cells releasing degradation facilitating factors. In certain embodiments, the effective size of the polymer carrier at pH~7.5 does not detectably change over 1 to 7 days, and remains within 50% of the original polymer size for at least several weeks. At pH-5, on the other hand, the polymer carrier preferably detectably degrades over 1 to 5 days, and is completely transformed into low molecular weight fragments within a two-week to several-month time frame. Polymer integrity in such tests can be measured, for example, by size exclusion HPLC. Although faster degradation may be in some cases preferable, in general it may be more desirable that the polymer degrades in cells with the rate that does not exceed the rate of metabolization or excretion of polymer fragments by the cells. In certain embodiments, the polymers and polymer biodegradation byproducts are biocompatible.

As used herein, the term “bioavailability” refers to the systemic availability (i.e., blood/plasma levels) of a given amount of oligonucleotide or compound administered to a subject. Bioavailability is an absolute term that indicates measurement of both the time (rate) and total amount (extent) of oligonucleotide or compound that reaches the general circulation from an administered dosage form.

As used herein, the term “drug release mechanism” refers to a linking moiety that is biocleavable/biodegradable under intracellular conditions, such that the cleavage of the linking moiety release the drug (e.g., oligonucleotides) in the intracellular environment. In some embodiments, the linking moiety is hydrolytically labile in water or in aqueous solutions including for example, body fluid such as blood, i.e., sensitive to hydrolysis at certain pHs. In some embodiments, the linking moiety is enzymatically labile, i.e., degradable by one or more enzymes. In some embodiments, the linking moiety is photo labile and is useful at the body surface and in many body cavities that are accessible to light. In some embodiments, the linking moiety is biocleavable under reducing conditions under which the activity of drug (e.g., oligonucleotides) is not affected.

Classes of drug molecules that can be used in the present disclosure include, but are not limited to, anti-cancer substances, radionuclides, vitamins, anti-AIDS substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, imaging agents.

As used herein the term “cytotoxic” means toxic to cells or a selected cell population (e.g., cancer cells). The toxic effect may result in cell death and/or lysis. In certain instances, the toxic effect may be a sublethal destructive effect on the cell, e.g., slowing or arresting cell growth. In order to achieve a cytotoxic effect, the drug or prodrug may be selected from a group consisting of a DNA damaging agent, a microtubule disrupting agent, or a cytotoxic protein or polypeptide, amongst others.

As used herein, the term “specific binding” or “specifically binds” refers to a non-random binding reaction between two molecules, such as for example between an antibody and an antigen. In some embodiments, the antibodies or antigen-binding fragments provided herein specifically bind to a target antigen with a binding affinity (K_{D}) of about 0.01 nM to about 100 nM, about 0.1 nM to about 100 nM, 0.01 nM to about 10 nM, about 0.1 nM to about 10 nM, 0.01 nM to about 1 nM, about 0.1 nM to about 1 nM or about 0.01 nM to about 0.1 nM) at pH 7.4. K_{D} as used herein refers to the ratio of the dissociation rate to the association rate (k_{off}/kₒₙ), may be determined using surface plasmon resonance methods for example using instrument such as Biacore.

As used herein, the term “tumor antigen” refers to an antigenic substance produced in tumor cells, i.e., it triggers an immune response in the host. Normal proteins in the body are not antigenic because of self-tolerance, a process in which self-reacting cytotoxic T lymphocytes (CTLs) and autoantibody-producing B lymphocytes are culled “centrally” in primary lymphatic tissue (BM) and “peripherally” in secondary lymphatic tissue (mostly thymus for T-cells and spleen/lymph nodes for B cells). Thus, any protein that is not exposed to the immune system triggers an immune response. This may include normal proteins that are well sequestered from the immune system, proteins that are normally produced in extremely small quantities, proteins that are normally produced only in certain stages of development, or proteins whose structure is modified due to mutation.

As used herein, the term “effective amount” refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, etc. For example, the effective amount of microparticles containing an antigen to be delivered to immunize an individual is the amount that results in an immune response sufficient to prevent infection with an organism having the administered antigen.

As used herein, “molecular weight” or “MW” of a polymer or polymeric carrier/scaffold or polymer conjugates refers to the weight average molecular weight unless otherwise specified.

The present disclosure is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C¹³ and C¹⁴.

The present disclosure is intended to include all isomers of the compound, which refers to and includes, optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers.

### Polymeric Carrier

The conjugates of the present disclosure are useful in biomedical applications, such as drug delivery and tissue engineering, and the polymeric carrier used in the conjugate of the present disclosure is biocompatible and biodegradable. In some embodiments, the polymeric carrier is a soluble polymer, nanoparticle, gel, liposome, micelle, suture, implant, etc.

In some embodiments, the polymeric carrier can have a weight average molecular weight Mw of from about 400 to about 3,000,000 Da, for example, from about 1,000 to about 2,000,000 Da, from about 1,000 to about 1,000,000 Da, from about 1,000 to about 900,000 Da, from about 1,000 to about 800,000 Da, from about 1,000 to about 700,000 Da, from about 1,000 to about 600,000 Da, from about 1,000 to about 500,000 Da, from about 1,000 to about 400,000 Da, from about 1,000 to about 300,000 Da, from about 1,000 to about 200,000 Da, from about 1,000 to about 100,000 Da, from about 1,000 to about 90,000 Da, from about 1,000 to about 80,000 Da, from about 1,000 to about 70,000 Da, from about 1,000 to about 60,000 Da, from about 1,000 to about 50,000 Da, from about 1,000 to about 40,000 Da, from about 1,000 to about 30,000 Da, from about 1,000 to about 20,000 Da, from about 1,000 to about 10,000 Da, from about 2,000 to about 10,000 Da, from about 3,000 to about 10,000 Da, from about 4,000 to about 10,000 Da, from about 5,000 to about 10,000 Da.

In some embodiments, the polymeric carrier used in the present disclosure is polyglycerol. In certain embodiments, the polymeric carrier used in the present disclosure is linear polyglycerol. In certain embodiments, the linear polyglycerol can have a weight average molecular weight Mw of from about 400 to about 3,000,000 Da, for example, from about 1,000 to about 2,000,000 Da, from about 1,000 to about 1,000,000 Da, from about 1,000 to about 900,000 Da, from about 1,000 to about 800,000 Da, from about 1,000 to about 700,000 Da, from about 1,000 to about 600,000 Da, from about 1,000 to about 500,000 Da, from about 1,000 to about 400,000 Da, from about 1,000 to about 300,000 Da, from about 1,000 to about 200,000 Da, from about 1,000 to about 100,000 Da, from about 1,000 to about 90,000 Da, from about 1,000 to about 80,000 Da, from about 1,000 to about 70,000 Da, from about 1,000 to about 60,000 Da, from about 1,000 to about 50,000 Da, from about 1,000 to about 40,000 Da, from about 1,000 to about 30,000 Da, from about 1,000 to about 20,000 Da, from about 1,000 to about 10,000 Da, from about 2,000 to about 10,000 Da, from about 3,000 to about 10,000 Da, from about 4,000 to about 10,000 Da, from about 5,000 to about 10,000 Da.

### Oligonucleotide

The term “oligonucleotide (ON)” is herein used interchangeably with “polynucleotide”, “nucleotide sequence”, and “nucleic acid”, and refers to a polymer containing at least two nucleotides. Nucleotides are the monomeric units of polynucleotide polymers. Natural nucleic acids have a deoxyribose- or ribose-phosphate backbone. A non-natural or synthetic polynucleotide is a polynucleotide that is polymerized in vitro or in a cell free system and contains the same or similar bases but may contain a backbone of a type other than the natural ribose or deoxyribose-phosphate backbone. Polynucleotides can be synthesized using any known technique in the art. Polynucleotide backbones known in the art include PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups on the nucleotide such as, but not limited to, amines, alcohols, thiols, carboxylates, and alkylhalides. The term base encompasses any of the known base analogs of DNA and RNA. The term polynucleotide includes deoxyribonucleic acid (DNA) and ribonucleic acid (RNA) and combinations of DNA, RNA and other natural and synthetic nucleotides.

DNA may be in form of cDNA, in vitro polymerized DNA, plasmid DNA, parts of a plasmid DNA, genetic material derived from a virus, linear DNA, vectors (P1, PAC, BAC, YAC, and artificial chromosomes), expression vectors, expression cassettes, chimeric sequences, recombinant DNA, chromosomal DNA, an oligonucleotide, anti-sense DNA, or derivatives of these groups. RNA may be in the form of messenger RNA (mRNA), in vitro polymerized RNA, recombinant RNA, oligonucleotide RNA, transfer RNA (tRNA), small nuclear RNA (snRNA), ribosomal RNA (rRNA), chimeric sequences, anti-sense RNA, interfering RNA, small interfering RNA (siRNA), microRNA (miRNA), ribozymes, external guide sequences, small non-messenger RNAs (snmRNA), untranslatedRNA (utRNA), snoRNAs (24-mers, modified snmRNA that act by an anti-sense mechanism), tiny non-coding RNAs (tncRNAs), small hairpin RNA (shRNA), or derivatives of these groups. In addition, DNA and RNA may be single, double, triple, or quadruple stranded. Double, triple, and quadruple stranded polynucleotide may contain both RNA and DNA or other combinations of natural and/or synthetic nucleic acids.

A blocking polynucleotide is a polynucleotide that interferes with the function or expression of DNA or RNA. Blocking polynucleotides are not translated into protein but their presence or expression in a cell alters the expression or function of cellular genes or RNA. Blocking polynucleotides cause the degradation of or inhibit the function or translation of a specific cellular RNA, usually an mRNA, in a sequence-specific manner. Inhibition of an RNA can thus effectively inhibit expression of a gene from which the RNA is transcribed. As used herein, a blocking polynucleotide may be selected from the list comprising: anti-sense oligonucleotide, RNA interference polynucleotide, dsRNA, siRNA, miRNA, hRNA, ribozyme, hammerhead ribozyme, external guide sequence (U.S. Pat. No. 5,962,426), snoRNA, triple-helix forming oligonucleotide RNA Polymerase II transcribed DNA encoding a blocking polynucleotide, RNA Polymerase III transcribed DNAs encoding a blocking polynucleotide. Blocking polynucleotide can be DNA, RNA, combination of RNA and DNA, or may contain non-natural or synthetic nucleotides. Blocking polynucleotides may be polymerized in vitro, they may be recombinant, contain chimeric sequences, or derivatives of these groups. A blocking polynucleotide may contain ribonucleotides, deoxyribonucleotides, synthetic nucleotides, or any suitable combination such that the target RNA or gene is inhibited.

An RNA interference (RNAi) polynucleotide is a molecule capable inducing RNA interference through interaction with the RNA interference pathway machinery of mammalian cells to degrade or inhibit translation of messenger RNA (mRNA) transcripts of a transgene in a sequence specific manner. Two primary RNAi polynucleotides are small (or short) interfering RNAs (siRNAs) and micro RNAs (miRNAs). However, other polynucleotides have been shown to mediate RNA interference. RNAi polynucleotides may be selected from the group comprising: siRNA, microRNA, double-strand RNA (dsRNA), short hairpin RNA (shRNA), and expression cassettes encoding RNA capable if inducing RNA interference. SiRNA comprises a double stranded structure typically containing 15-50 base pairs and preferably 21-25 base pairs and having a nucleotide sequence identical (perfectly complementary) or nearly identical (partially complementary) to a coding sequence in an expressed target gene or RNA within the cell. An siRNA may have dinucleotide 3′ overhangs. An siRNA may be composed of two annealed polynucleotides or a single polynucleotide that forms a hairpin structure. An siRNA molecule of the present disclosure comprises a sense region and an antisense region. In one embodiment, the siRNA of the conjugate is assembled from two oligonucleotide fragments, wherein one fragment comprises the nucleotide sequence of the antisense strand of the siRNA molecule and a second fragment comprises nucleotide sequence of the sense region of the siRNA molecule. In another embodiment, the sense strand is connected to the antisense strand via a linker molecule, such as a polynucleotide linker or a non-nucleotide linker. MicroRNAs (miRNAs) are small noncoding RNA gene products about 22 nt long that direct destruction or translational repression of their mRNA targets. If the complementarity between the miRNA and the target mRNA is partial, then translation of the target mRNA is repressed, whereas if complementarity is extensive, the target mRNA is cleaved. For miRNAs, the complex binds to target sites usually located in the 3′ UTR of mRNAs that typically share only partial homology with the miRNA. A “seed region”—a stretch of about 7 consecutive nucleotides on the 5′ end of the miRNA that forms perfect base pairing with its target—plays a key role in miRNA specificity. Binding of the RISC/miRNA complex to the mRNA can lead to either the repression of protein translation or cleavage and degradation of the mRNA. Recent data indicate that mRNA cleavage happens preferentially if there is perfect homology along the whole length of the miRNA and its target instead of showing perfect base-pairing only in the seed region (Pillai et al. 2007).

Antisense oligonucleotide comprises a polynucleotide containing sequence that is complimentary to a sequence present in a target mRNA. The antisense oligonucleotide binds to (base pairs with) mRNA in a sequence specific manner. This binding can prevent other cellular enzymes from binding to the mRNA, thereby leading to inhibition of translation of the mRNA or degradation of the mRNA.

External guide sequences are short antisense oligoribonucleotides that induce RNase P-mediated cleavage of a target RNA by forming a precursor tRNA-like complex (U.S. Pat. No. 5,624,824).

Ribozymes are typically RNA oligonucleotides that contain sequence complementary to the target messenger RNA and an RNA sequence that acts as an enzyme to cleave the messenger RNA. Cleavage of the mRNA prevents translation.

An oligonucleotide that forms the blocking polynucleotide can include a terminal cap moiety at the 5′-end, the 3′-end, or both of the 5′ and 3′ ends. The cap moiety can be, but is not limited to, an inverted deoxy abasic moiety, an inverted deoxy thymidine moiety, a thymidine moiety, or 3′ glyceryl modification.

RNA polymerase II and III transcribed DNAs can be transcribed in the cell to produce small hairpin RNAs that can function as siRNA, separate sense and anti-sense strand linear siRNAs, ribozymes, or linear RNAs that can function as antisense RNA. RNA polymerase III transcribed DNAs contain promoters selected from the list comprising: U6 promoters, H1 promoters, and tRNA promoters. RNA polymerase II promoters include U1, U2, U4, and U5 promoters, snRNA promoters, microRNA promoters, and mRNA promoters.

Lists of known miRNA sequences can be found in databases maintained by research organizations such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Pei et al. 2006, Reynolds et al. 2004, Khvorova et al. 2003, Schwarz et al. 2003, Ui-Tei et al. 2004, Heale et al. 2005, Chalk et al. 2004, Amarzguioui et al. 2004).

The polynucleotides of the present disclosure can be chemically modified. The use of chemically modified polynucleotide can improve various properties of the polynucleotide including, but not limited to: resistance to nuclease degradation in vivo, cellular uptake, activity, and sequence-specific hybridization. Non-limiting examples of such chemical modifications include: phosphorothioate internucleotide linkages, 2′-O-methyl ribonucleotides, 2′-deoxy-2′-fluoro ribonucleotides, 2′-deoxy ribonucleotides, “universal base” nucleotides, 5-C-methyl nucleotides, and inverted deoxyabasic residue incorporation. These chemical modifications, when used in various polynucleotide constructs, are shown to preserve polynucleotide activity in cells while at the same time, dramatically increasing the serum stability of these compounds. Chemically modified siRNA can also minimize the possibility of activating interferon activity in humans.

In one embodiment, the chemically-modified RNAi polynucleotide of the present disclosure comprises a duplex having two strands, one or both of which can be chemically-modified, wherein each strand is about 19 to about 29 (e.g., about 19, 20, 21, 22, 23, 24, 25, 26, 27, 28, or 29) nucleotides. In one embodiment, an RNAi polynucleotide of the present disclosure comprises one or more modified nucleotides while maintaining the ability to mediate RNAi inside a cell or reconstituted in vitro system. An RNAi polynucleotide can be modified wherein the chemical modification comprises one or more (e.g., about 1, 2, 3, 4, 5, 6, 7, 8, 9, 10, or more) of the nucleotides. An RNAi polynucleotide of the present disclosure can comprise modified nucleotides as a percentage of the total number of nucleotides present in the RNAi polynucleotide. As such, an RNAi polynucleotide of the present disclosure can generally comprise modified nucleotides from about 5 to about 100% of the nucleotide positions (e.g., 5%, 10%, 15%, 20%, 25%, 30%, 35%, 40%, 45%, 50%, 55%, 60%, 65%, 70%, 75%, 80%, 85%, 90%, 95% or 100% of the nucleotide positions). The actual percentage of modified nucleotides present in a given RNAi polynucleotide depends on the total number of nucleotides present in the RNAi polynucleotide. If the RNAi polynucleotide is single stranded, the percent modification can be based upon the total number of nucleotides present in the single stranded RNAi polynucleotide. Likewise, if the RNAi polynucleotide is double stranded, the percent modification can be based upon the total number of nucleotides present in the sense strand, antisense strand, or both the sense and antisense strands. In addition, the actual percentage of modified nucleotides present in a given RNAi polynucleotide can also depend on the total number of purine and pyrimidine nucleotides present in the RNAi polynucleotide. For example, wherein all pyrimidine nucleotides and/or all purine nucleotides present in the RNAi polynucleotide are modified.

An RNAi polynucleotide modulates expression of RNA encoded by a gene. Because multiple genes can share some degree of sequence homology with each other, an RNAi polynucleotide can be designed to target a class of genes with sufficient sequence homology. Thus, an RNAi polynucleotide can contain sequence that has complementarity to sequences that are shared amongst different gene targets or are unique for a specific gene target. Therefore, the RNAi polynucleotide can be designed to target conserved regions of an RNA sequence having homology between several genes thereby targeting several genes in a gene family (e.g., different gene isoforms, splice variants, mutant genes etc.). In another embodiment, the RNAi polynucleotide can be designed to target a sequence that is unique to a specific RNA sequence of a single gene.

The term “complementarity” refers to the ability of a polynucleotide to form hydrogen bond(s) with another polynucleotide sequence by either traditional Watson-Crick or other non-traditional types. In reference to the polynucleotide molecules of the present disclosure, the binding free energy for a polynucleotide molecule with its target (effector binding site) or complementary sequence is sufficient to allow the relevant function of the polynucleotide to proceed, e.g., enzymatic mRNA cleavage or translation inhibition. Determination of binding free energies for nucleic acid molecules is well known in the art (Frier et al. 1986, Turner et al. 1987). A percent complementarity indicates the percentage of bases, in a contiguous strand, in a first polynucleotide molecule which can form hydrogen bonds (e.g., Watson-Crick base pairing) with a second polynucleotide sequence (e.g., 5, 6, 7, 8, 9, 10 out of 10 being 50%, 60%, 70%, 80%, 90%, and 100% complementary). Perfectly complementary means that all the bases, in a contiguous strand of a polynucleotide sequence will hydrogen bond with the same number of contiguous bases in a second polynucleotide sequence.

By “inhibit, down-regulate, or knockdown gene expression”, it is meant that the expression of the gene, as measured by the level of RNA transcribed from the gene, or the level of polypeptide, protein or protein subunit translated from the RNA, is reduced below that observed in the absence of the polymeric scaffold provided herein.

Inhibition, down-regulation, or knockdown of gene expression, with an oligonucleotide delivered by the polymeric scaffold provided herein, is preferably below that level observed in the presence of a control inactive nucleic acid, a nucleic acid with scrambled sequence or with inactivating mismatches, or in absence of conjugation of the polynucleotide to the masked polymer.

A delivered oligonucleotide can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

An oligonucleotide can be delivered to a cell to express an exogenous nucleotide sequence, to inhibit, eliminate, augment, or alter expression of an endogenous nucleotide sequence, or to affect a specific physiological characteristic not naturally associated with the cell.

Oligonucleotides may contain an expression cassette coded to express a whole or partial protein, or RNA. An expression cassette refers to a natural or recombinantly produced polynucleotide that is capable of expressing a sequence. The term recombinant as used herein refers to a polynucleotide molecule that is comprised of segments of polynucleotide joined together by means of molecular biological techniques. The cassette contains the coding region of the gene of interest along with any other sequences that affect expression of the sequence of interest. An expression cassette typically includes a promoter (allowing transcription initiation), and a transcribed sequence. Optionally, the expression cassette may include, but is not limited to: transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include, but is not limited to, translation termination signals, a polyadenosine sequence, internal ribosome entry sites (IRES), and non-coding sequences.

The oligonucleotide may contain sequences that do not serve a specific function in the target cell but are used in the generation of the polynucleotide. Such sequences include, but are not limited to, sequences required for replication or selection of the polynucleotide in a host organism.

The term “gene” generally refers to a polynucleotide sequence that comprises coding sequences necessary for the production of a therapeutic polynucleotide (e.g., ribozyme) or a polypeptide or precursor. The polypeptide can be encoded by a full-length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term gene encompasses both cDNA and genomic forms of a gene. A genomic form or clone of a gene contains the coding region interrupted with non-coding sequences termed introns, intervening regions, or intervening sequences. Introns are segments of a gene which are transcribed into nuclear RNA. Introns may contain regulatory elements such as enhancers. Introns are removed or spliced out from the nuclear or primary transcript; introns therefore are absent in the mature RNA transcript. The messenger RNA (mRNA) functions during translation to specify the sequence or order of amino acids in a nascent polypeptide. A gene may also includes other regions or sequences including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotides) or at distant sites (more than 10,000 nucleotides). These non-coding sequences influence the level or rate of transcription and/or translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, nuclear transport, and immunogenicity. One example of covalent modification of nucleic acid involves the action of LABELIT® reagents (Minis Corporation, Madison, WI).

As used herein, the term “gene expression” refers to the process of converting genetic information encoded in a gene into RNA (e.g., small RNA, siRNA, mRNA, rRNA, tRNA, or snRNA) through transcription of a deoxyribonucleic gene (e.g., via the enzymatic action of an RNA polymerase), and for protein encoding genes, into protein through translation of mRNA. Gene expression can be regulated at many stages in the process. Up-regulation or activation refers to regulation that increases the production of gene expression products (i.e., RNA or protein), while down-regulation or repression refers to regulation that decrease production. Molecules (e.g., transcription factors) that are involved in up-regulation or down-regulation are often called activators and repressors, respectively.

The oligonucleotide may be used can be therapeutic. The delivery of a oligonucleotide for therapeutic purposes is commonly called gene therapy. The delivery of a oligonucleotide can lead to modification of the genetic material present in the target cell. The term “stable transfection” or “stably transfected” generally refers to the introduction and integration of an exogenous polynucleotide into the genome of the transfected cell. The term “stable transfectant” refers to a cell which has stably integrated the oligonucleotide into the genomic DNA. Stable transfection can also be obtained by using episomal vectors that are replicated during the eukaryotic cell division (e.g., plasmid DNA vectors containing a papilloma virus origin of replication, artificial chromosomes). The term “transient transfection” or “transiently transfected” refers to the introduction of a polynucleotide into a cell where the polynucleotide does not integrate into the genome of the transfected cell. If the oligonucleotide contains an expressible gene, then the expression cassette is subject to the regulatory controls that govern the expression of endogenous genes in the chromosomes. The term “transient transfectant” refers to a cell which has taken up a polynucleotide but has not integrated the polynucleotide into its genomic DNA.

The oligonucleotide-polymer conjugate is formed by covalently linking the oligonucleotide to the polymer. The polymer is polymerized or modified such that it contains a reactive group A. The oligonucleotide is also polymerized or modified such that it contains a reactive group B. Reactive groups A and B are chosen such that they can be linked via a reversible covalent linkage using methods known in the art.

In certain embodiment, the oligonucleotide is siRNA. In certain embodiment, the oligonucleotide is antisense oligonucleotides. In certain embodiments, the oligonucleotide is mRNA. In certain embodiments, the oligonucleotide is saRNA. In certain embodiment, the oligonucleotide is miRNA. In certain embodiment, the oligonucleotide is shRNA. In certain embodiment, the oligonucleotide is CRISPER/Cas9 complex.

In some embodiment, the oligonucleotide is siRNA. In certain embodiments, the siRNA is selected from the group consisting of siRNA_1-100 shown as below.

| | | | SEQ | | | SEQ |
|---|---|---|---|---|---|---|
| siRNA_# | SS No. | Sequence (5′-3′) | ID NO. | AS No. | Sequence (5′-3′) | ID NO. |
| 1 | SS1 | CACACUGCAAGUGGACAUCAA | 1 | AS1 | GAUGUCCACUUGCAGUGUGUU | 101 |
| 2 | SS2 | CACAUCAGAGCUCCGAGAAGC | 2 | AS2 | UUCUCGGAGCUCUGAUGUGUU | 102 |
| 3 | SS3 | CACUACUGUAGUUAGAUCUAU | 3 | AS3 | AGAUCUAACUACAGUAGUGUU | 103 |
| 4 | SS4 | GCCUGUCUGCACUAUUCCUUU | 4 | AS4 | AGGAAUAGUGCAGACAGGCAG | 104 |
| 5 | SS5 | GAGACGGAUCUCUCUCCGACC | 5 | AS5 | UCGGAGAGAGAUCCGUCUCCU | 105 |
| 6 | SS6 | GGCCCUGUACAACCAGCAUAA | 6 | AS6 | AUGCUGGUUGUACAGGGCCAG | 106 |
| 7 | SS7 | GAACACUACUGUAGUUAGAUC | 7 | AS7 | UCUAACUACAGUAGUGUUCCC | 107 |
| 8 | SS8 | GCCCAUCUAGGUUAUUUCCGU | 8 | AS8 | GGAAAUAACCUAGAUGGGCGC | 108 |
| 9 | SS9 | GGACACCAACUAUUGCUUCAG | 9 | AS9 | GAAGCAAUAGUUGGUGUCCAG | 109 |
| 10 | SS10 | GCGGUACCUGAACCCGUGUUG | 10 | AS10 | ACACGGGUUCAGGUACCGCUU | 110 |
| 11 | SS11 | GCCCCUACAUUUGGAGCCUGG | 11 | AS11 | AGGCUCCAAAUGUAGGGGCAG | 111 |
| 12 | SS12 | GCAAGACUAUCGACAUGGAGC | 12 | AS12 | UCCAUGUCGAUAGUCUUGCAG | 112 |
| 13 | SS13 | GACUCGCCAGAGUGGUUAUCU | 13 | AS13 | AUAACCACUCUGGCGAGUCGC | 113 |
| 14 | SS14 | GCCACAGAUCCCCUAUUCAAG | 14 | AS14 | UGAAUAGGGGAUCUGUGGCAG | 114 |
| 15 | SS15 | GGUGGAAACCCACAACGAAAU | 15 | AS15 | UUCGUUGUGGGUUUCCACCAU | 115 |
| 16 | SS16 | GCCGUGGAGGGGAAAUUGAGG | 16 | AS16 | UCAAUUUCCCCUCCACGGCUC | 116 |
| 17 | SS17 | GCCCCAUUAAAGAUGGAGAGA | 17 | AS17 | UCUCCAUCUUUAAUGGGGCCC | 117 |
| 18 | SS18 | GUGGGGAACACUACUGUAGUU | 18 | AS18 | CUACAGUAGUGUUCCCCACUG | 118 |
| 19 | SS19 | GCAGCUGUCCAACAUGAUCGU | 19 | AS19 | GAUCAUGUUGGACAGCUGCUC | 119 |
| 20 | SS20 | GCCCUGUACAACCAGCAUAAC | 20 | AS20 | UAUGCUGGUUGUACAGGGCCA | 120 |
| 21 | SS21 | GAGAGGACUGCGGAUCUCUGU | 21 | AS21 | AGAGAUCCGCAGUCCUCUCUC | 121 |
| 22 | SS22 | GGGAACACUACUGUAGUUAGA | 22 | AS22 | UAACUACAGUAGUGUUCCCCA | 122 |
| 23 | SS23 | GUGGAAACCCACAACGAAAUC | 23 | AS23 | UUUCGUUGUGGGUUUCCACCA | 123 |
| 24 | SS24 | GCAACACUCUGAGAUGCAGGG | 24 | AS24 | CUGCAUCUCAGAGUGUUGCUA | 124 |
| 25 | SS25 | CGGCAGCUGUACAUUGACUUC | 25 | AS25 | AGUCAAUGUACAGCUGCCGCA | 125 |
| 26 | SS26 | GACACCAACUAUUGCUUCAGC | 26 | AS26 | UGAAGCAAUAGUUGGUGUCCA | 126 |
| 27 | SS27 | GCAGUACAGCAAGGUCCUGGC | 27 | AS27 | CAGGACCUUGCUGUACUGCGU | 127 |
| 28 | SS28 | GCUGUCCAACAUGAUCGUGCG | 28 | AS28 | CACGAUCAUGUUGGACAGCUG | 128 |
| 29 | SS29 | CCAUAGCAACACUCUGAGAUG | 29 | AS29 | UCUCAGAGUGUUGCUAUGGUG | 129 |
| 30 | SS30 | GUUGUGCGGCAGUGGUUGAGC | 30 | AS30 | UCAACCACUGCCGCACAACUC | 130 |
| 31 | SS31 | GACCUGGCCACCAUUCAUGGC | 31 | AS31 | CAUGAAUGGUGGCCAGGUCAC | 131 |
| 32 | SS32 | GCAGAGUACACACAGCAUAUA | 32 | AS32 | UAUGCUGUGUGUACUCUGCUU | 132 |
| 33 | SS33 | GCGGCAGCUGUACAUUGACUU | 33 | AS33 | GUCAAUGUACAGCUGCCGCAC | 133 |
| 34 | SS34 | GUCUGCACUAUUCCUUUGCCC | 34 | AS34 | GCAAAGGAAUAGUGCAGACAG | 134 |
| 35 | SS35 | GAGGUUAAGGGAGGAGUUCCU | 35 | AS35 | GAACUCCUCCCUUAACCUCUC | 135 |
| 36 | SS36 | GCAUCCUAGACCCUUUCUCCU | 36 | AS36 | GAGAAAGGGUCUAGGAUGCGC | 136 |
| 37 | SS37 | GCAACAAUUCCUGGCGAUACC | 37 | AS37 | UAUCGCCAGGAAUUGUUGCUG | 137 |
| 38 | SS38 | GCAGGGAUAACACACUGCAAG | 38 | AS38 | UGCAGUGUGUUAUCCCUGCUG | 138 |
| 39 | SS39 | GCACUGUUGAAGUGCCUUACA | 39 | AS39 | UAAGGCACUUCAACAGUGCCC | 139 |
| 40 | SS40 | CACAACGAAAUCUAUGACAAG | 40 | AS40 | UGUCAUAGAUUUCGUUGUGGG | 140 |
| 41 | SS41 | GACCUCAGCUUUCCCUCGAGG | 41 | AS41 | UCGAGGGAAAGCUGAGGUCCU | 141 |
| 42 | SS42 | GAGGACUGCGGAUCUCUGUGU | 42 | AS42 | ACAGAGAUCCGCAGUCCUCUC | 142 |
| 43 | SS43 | GAGUGGUUAUCUUUUGAUGUC | 43 | AS43 | CAUCAAAAGAUAACCACUCUG | 143 |
| 44 | SS44 | GGAACACUACUGUAGUUAGAU | 44 | AS44 | CUAACUACAGUAGUGUUCCCC | 144 |
| 45 | SS45 | GAUCGUGCGCUCCUGCAAGUG | 45 | AS45 | CUUGCAGGAGCGCACGAUCAU | 145 |
| 46 | SS46 | GCACCUUGGGCACUGUUGAAG | 46 | AS46 | UCAACAGUGCCCAAGGUGCUC | 146 |
| 47 | SS47 | GCUUCAGCUCCACGGAGAAGA | 47 | AS47 | UUCUCCGUGGAGCUGAAGCAA | 147 |
| 48 | SS48 | GCAGCUGUACAUUGACUUCCG | 48 | AS48 | GAAGUCAAUGUACAGCUGCCG | 148 |
| 49 | SS49 | GUUCUUCAACACAUCAGAGCU | 49 | AS49 | CUCUGAUGUGUUGAAGAACAU | 149 |
| 50 | SS50 | CUCCUCCAGGAGACGGAUCUC | 50 | AS50 | GAUCCGUCUCCUGGAGGAGAA | 150 |
| 51 | SS51 | CCGCGUGCUAAUGGUGGAAAC | 51 | AS51 | UUCCACCAUUAGCACGCGGGU | 151 |
| 52 | SS52 | CGCAUCCUAGACCCUUUCUCC | 52 | AS52 | AGAAAGGGUCUAGGAUGCGCG | 152 |
| 53 | SS53 | CUGGCCACCAUUCAUGGCAUG | 53 | AS53 | UGCCAUGAAUGGUGGCCAGGU | 153 |
| 54 | SS54 | CUCCAGGAGACGGAUCUCUCU | 54 | AS54 | AGAGAUCCGUCUCCUGGAGGA | 154 |
| 55 | SS55 | CCACAGAUCCCCUAUUCAAGA | 55 | AS55 | UUGAAUAGGGGAUCUGUGGCA | 155 |
| 56 | SS56 | CCAGGAGACGGAUCUCUCUCC | 56 | AS56 | AGAGAGAUCCGUCUCCUGGAG | 156 |
| 57 | SS57 | CCUGGCGAUACCUCAGCAACC | 57 | AS57 | UUGCUGAGGUAUCGCCAGGAA | 157 |
| 58 | SS58 | GGCUGUAUUUAAGGACACCCG | 58 | AS58 | GGUGUCCUUAAAUACAGCCCC | 158 |
| 59 | SS59 | GGUGAGGAAACAAGCCCAGAG | 59 | AS59 | CUGGGCUUGUUUCCUCACCUU | 159 |
| 60 | SS60 | GGAUAGUGAAGAAGACAAUAA | 60 | AS60 | AUUGUCUUCUUCACUAUCCCC | 160 |
| 61 | SS61 | GGAGAGAGGACUGCGGAUCUC | 61 | AS61 | GAUCCGCAGUCCUCUCUCCAU | 161 |
| 62 | SS62 | GGACUAUCCACCUGCAAGACU | 62 | AS62 | UCUUGCAGGUGGAUAGUCCCG | 162 |
| 63 | SS63 | GAGAGAGGACUGCGGAUCUCU | 63 | AS63 | AGAUCCGCAGUCCUCUCUCCA | 163 |
| 64 | SS64 | GAUGCAGGGACUCUGAUAACA | 64 | AS64 | UUAUCAGAGUCCCUGCAUCUC | 164 |
| 65 | SS65 | GACUAUCCACCUGCAAGACUA | 65 | AS65 | GUCUUGCAGGUGGAUAGUCCC | 165 |
| 66 | SS66 | GACACGCAGUACAGCAAGGUC | 66 | AS66 | CCUUGCUGUACUGCGUGUCCA | 166 |
| 67 | SS67 | GGGGCCCCAUUAAAGAUGGAG | 67 | AS67 | CCAUCUUUAAUGGGGCCCCAG | 167 |
| 68 | SS68 | GUGACAGCAGGGAUAACACAC | 68 | AS68 | GUGUUAUCCCUGCUGUCACAG | 168 |
| 69 | SS69 | GCAGGGACUCUGAUAACACCC | 69 | AS69 | GUGUUAUCAGAGUCCCUGCAU | 169 |
| 70 | SS70 | CGCGUGCUAAUGGUGGAAACC | 70 | AS70 | UUUCCACCAUUAGCACGCGGG | 170 |
| 71 | SS71 | GUGGAUCCACGAGCCCAAGGG | 71 | AS71 | CUUGGGCUCGUGGAUCCACUU | 171 |
| 72 | SS72 | GCGUGCUAAUGGUGGAAACCC | 72 | AS72 | GUUUCCACCAUUAGCACGCGG | 172 |
| 73 | SS73 | CUGAUAACACCCAUUUUAAAG | 73 | AS73 | UUAAAAUGGGUGUUAUCAGAG | 173 |
| 74 | SS74 | AACCCACAACGAAAUCUAUGA | 74 | AS74 | AUAGAUUUCGUUGUGGGUUUC | 174 |
| 75 | SS75 | CUACUGUAGUUAGAUCUAUUU | 75 | AS75 | AUAGAUCUAACUACAGUAGUG | 175 |
| 76 | SS76 | CCUGAACCCGUGUUGCUCUCC | 76 | AS76 | AGAGCAACACGGGUUCAGGUA | 176 |
| 77 | SS77 | CACCAUAGCAACACUCUGAGA | 77 | AS77 | UCAGAGUGUUGCUAUGGUGAC | 177 |
| 78 | SS78 | CACCCACCUUCUGGUACCAGA | 78 | AS78 | UGGUACCAGAAGGUGGGUGGU | 178 |
| 79 | SS79 | CACUGCAAGUGGACAUCAACG | 79 | AS79 | UUGAUGUCCACUUGCAGUGUG | 179 |
| 80 | SS80 | GCACUAUUCCUUUGCCCGGCA | 80 | AS80 | CCGGGCAAAGGAAUAGUGCAG | 180 |
| 81 | SS81 | CGAGAAGCGGUACCUGAACCC | 81 | AS81 | GUUCAGGUACCGCUUCUCGGA | 181 |
| 82 | SS82 | UGGCCACCAUUCAUGGCAUGA | 82 | AS82 | AUGCCAUGAAUGGUGGCCAGG | 182 |
| 83 | SS83 | UACCUGAACCCGUGUUGCUCU | 83 | AS83 | AGCAACACGGGUUCAGGUACC | 183 |
| 84 | SS84 | CCUCCCGCAAAGACUUUUCCC | 84 | AS84 | GAAAAGUCUUUGCGGGAGGCC | 184 |
| 85 | SS85 | CUGUUGAAGUGCCUUACAUUA | 85 | AS85 | AUGUAAGGCACUUCAACAGUG | 185 |
| 86 | SS86 | CACCAUUCAUGGCAUGAACCG | 86 | AS86 | GUUCAUGCCAUGAAUGGUGGC | 186 |
| 87 | SS87 | GUACAUUGACUUCCGCAAGGA | 87 | AS87 | CUUGCGGAAGUCAAUGUACAG | 187 |
| 88 | SS88 | UGACAGCAGGGAUAACACACU | 88 | AS88 | UGUGUUAUCCCUGCUGUCACA | 188 |
| 89 | SS89 | CCUGGACACCAACUAUUGCUU | 89 | AS89 | GCAAUAGUUGGUGUCCAGGGC | 189 |
| 90 | SS90 | GACUUCCGCAAGGACCUCGGC | 90 | AS90 | CGAGGUCCUUGCGGAAGUCAA | 190 |
| 91 | SS91 | CACGUGGAGCUGUACCAGAAA | 91 | AS91 | UCUGGUACAGCUCCACGUGCU | 191 |
| 92 | SS92 | CCAGAAAUACAGCAACAAUUC | 92 | AS92 | AUUGUUGCUGUAUUUCUGGUA | 192 |
| 93 | SS93 | CGCAGUACAGCAAGGUCCUGG | 93 | AS93 | AGGACCUUGCUGUACUGCGUG | 193 |
| 94 | SS94 | CCAUCUAGGUUAUUUCCGUGG | 94 | AS94 | ACGGAAAUAACCUAGAUGGGC | 194 |
| 95 | SS95 | CAGAGUGGUUAUCUUUUGAUG | 95 | AS95 | UCAAAAGAUAACCACUCUGGC | 195 |
| 96 | SS96 | CCUACCUUUUGCCGGGAGACC | 96 | AS96 | UCUCCCGGCAAAAGGUAGGAG | 196 |
| 97 | SS97 | CAGCAUAUAUAUGUUCUUCAA | 97 | AS97 | GAAGAACAUAUAUAUGCUGUG | 197 |
| 98 | SS98 | GCGGAUCUCUGUGUCAUUGGG | 98 | AS98 | CAAUGACACAGAGAUCCGCAG | 198 |
| 99 | SS99 | CCCCUACAUUUGGAGCCUGGA | 99 | AS99 | CAGGCUCCAAAUGUAGGGGCA | 199 |
| 100 | SS100 | CCCACAACGAAAUCUAUGACA | 100 | AS100 | UCAUAGAUUUCGUUGUGGGUU | 200 |

In some embodiments, the oligonucleotide is a modified siRNA. In certain embodiments, the sense strand sequence of the siRNA is shown as SEQ ID NO: 201, and antisense strand sequence of the siRNA is shown as SEQ ID NO: 202. In certain embodiments, the sense strand sequence of the siRNA is shown as SEQ ID NO: 203, and antisense strand sequence of the siRNA is shown as SEQ ID NO: 204. In certain embodiments, the sense strand sequence of the siRNA is shown as SEQ ID NO: 205, and antisense strand sequence of the siRNA is shown as SEQ ID NO: 206. In certain embodiments, the sense strand sequence of the siRNA is shown as SEQ ID NO: 207, and antisense strand sequence of the siRNA is shown as SEQ ID NO: 208. In certain embodiments, the sense strand sequence of the siRNA is shown as SEQ ID NO: 209, and antisense strand sequence of the siRNA is shown as SEQ ID NO: 210. In certain embodiments, the sense strand sequence of the siRNA is shown as SEQ ID NO: 211, and antisense strand sequence of the siRNA is shown as SEQ ID NO: 212. In certain embodiments, the sense strand sequence of the siRNA is shown as SEQ ID NO: 213, and antisense strand sequence of the siRNA is shown as SEQ ID NO: 214. In certain embodiments, the sense strand sequence of the siRNA is shown as SEQ ID NO: 215, and antisense strand sequence of the siRNA is shown as SEQ ID NO: 216. In certain embodiments, the sense strand sequence of the siRNA is shown as SEQ ID NO: 217, and antisense strand sequence of the siRNA is shown as SEQ ID NO: 218. In certain embodiments, the sense strand sequence of the siRNA is shown as SEQ ID NO: 219, and antisense strand sequence of the siRNA is shown as SEQ ID NO: 220. In certain embodiments, the sense strand sequence of the siRNA is shown as SEQ ID NO: 221, and antisense strand sequence of the siRNA is shown as SEQ ID NO: 222. In certain embodiments, the sense strand sequence of the siRNA is shown as SEQ ID NO: 223, and antisense strand sequence of the siRNA is shown as SEQ ID NO: 224. In certain embodiments, the sense strand sequence of the siRNA is shown as SEQ ID NO: 225, and antisense strand sequence of the siRNA is shown as SEQ ID NO: 226. In certain embodiments, the sense strand sequence of the siRNA is shown as SEQ ID NO: 227, and antisense strand sequence of the siRNA is shown as SEQ ID NO: 228. In certain embodiments, the sense strand sequence of the siRNA is shown as SEQ ID NO: 229, and antisense strand sequence of the siRNA is shown as SEQ ID NO: 230. In certain embodiments, the sense strand sequence of the siRNA is shown as SEQ ID NO: 231, and antisense strand sequence of the siRNA is shown as SEQ ID NO: 232. In certain embodiments, the sense strand sequence of the siRNA is shown as SEQ ID NO: 233, and antisense strand sequence of the siRNA is shown as SEQ ID NO: 234.

In some embodiments, the oligonucleotide is an antisense oligonucleotide (ASO). In certain embodiments, the sequence of the antisense oligonucleotide is shown as any one of SEQ ID Nos: 235-257.

In some embodiments, the oligonucleotide is a phosphorodiamidate morpholino oligomer (PMO). In certain embodiments, the sequence of the phosphorodiamidate morpholino oligomer is shown as any one of SEQ ID Nos: 258-263.

In some embodiments, the oligonucleotide silences the TGFβ. In certain embodiment, the oligonucleotide is siRNA silencing the TGFβ. In certain embodiments, the oligonucleotide is an antisense oligonucleotide (ASO) silencing the TGFβ. In certain embodiments, the oligonucleotide is mRNA silencing the TGFβ. In certain embodiments, the oligonucleotide is saRNA silencing the TGFβ. In certain embodiments, the oligonucleotide is miRNA silencing the TGFβ. In certain embodiments, the oligonucleotide is shRNA silencing the TGFβ. In certain embodiments, the oligonucleotide is CRISPER/Cas9 complex silencing the TGFβ. In certain embodiments, the oligonucleotide is phosphorodiamidate morpholino oligomer (PMO) silencing the TGFβ.

The modified oligonucleotides show a significantly increased inhibition on TGFβ expression and activity, respectively, which results in an improved prevention and/or treatment of a malignant or benign tumor, an immunologic disease, fibrosis, eye disease such as glaucoma or posterior capsular opacification (PCO), CNS disease hair loss etc. The oligonucleotides of the present disclosure target TGFβ linked diseases either by hybridization with TGFβ mRNA, preferably TGFβ 1, TGFβ2, or TGFβ3, alternatively, TGFβl, TGFβ2, and/or TGFβ3 mRNAs. An oligonucleotide inhibiting the expression of TGFβ1, TGFβ2, and TGFβ3 is defined as pan-specific oligonucleotide.

### Endosomal-Lysosomal Escape Moiety

### Cell Penetrating Peptide (CPP)

In some embodiments, endosomal-lysosomal escape moiety described herein may comprise one or more cell penetrating peptides (CPPs).

The term “cell penetrating peptides” is generally used to designate short peptides that are able to transport different types of cargo molecules across plasma membrane, and, thus, facilitate cellular uptake of various molecular cargoes (from nanosize particles to small chemical molecules and large fragments of DNA). “Cellular internalization” of the cargo molecule linked to the cell penetrating peptide generally means transport of the cargo molecule across the plasma membrane and thus entry of the cargo molecule into the cell. Depending on the particular case, the cargo molecule can, then, be released in the cytoplasm, directed to an intracellular organelle, or further presented at the cell surface. Cell penetrating ability, or internalization, of the cell penetrating peptide or conjugate comprising said cell penetrating peptide, can be checked by standard methods known to one skilled in the art, including flow cytometry or fluorescence microscopy of live and fixed cells, immunocytochemistry of cells transduced with said peptide or complex, and Western blot.

Cell penetrating peptides typically have an amino acid composition that either contains a high relative abundance of positively charged amino acids such as lysine or arginine or have a sequence that contains an alternating pattern of polar/charged amino acids and non-polar, hydrophobic amino acids. These two types of structures are referred to as polycationic or amphipathic, respectively. Cell penetrating peptides are of different sizes, amino acid sequences, and charges but all CPPs have a common characteristic that is the ability to translocate the plasma membrane and facilitate the delivery of various molecular cargoes to the cytoplasm or to an organelle of a cell. At present, the theories of CPP translocation distinguish three main entry mechanisms: direct penetration in the membrane, endocytosis-mediated entry, and translocation through the formation of a transitory structure. Cell penetrating peptides have found numerous applications in medicine as drug delivery agents in the treatment of different diseases including cancer and virus inhibitors, as well as contrast agents for cell labeling and imaging.

Typically, cell penetrating peptides are peptides of 8 to 50 residues that have the ability to cross the cell membrane and enter into most cell types. Alternatively, they are also called protein transduction domain (PTDs) reflecting their origin as occurring in natural proteins. Frankel and Pabo simultaneously to Green and Lowenstein described the ability of the trans-activating transcriptional activator from the human immunodeficiency virus 1 (HIV-TAT) to penetrate into cells (Frankel, A. D. and C. O. Pabo, Cellular uptake of the tat protein from human immunodeficiency virus. Cell, 1988. 55(6): p. 1189-93). In 1991, transduction into neural cells of the Antennapedia homeodomain (DNA-binding domain) from Drosophila melanogaster was described (Joliot, A., et al., Antennapedia homeobox peptide regulates neural morphogenesis. Proc Natl Acad Sci USA, 1991. 88(5): p. 1864-8). In 1994, the first 16-mer peptide CPP called Penetratin, having the amino acid sequence RQIKIYFQNRRMKWKK was characterized from the third helix of the homeodomain of Antennapedia (Derossi, D., et al., The third helix of the Antennapedia homeodomain translocates through biological membranes. J Biol Chem, 1994. 269(14): p. 10444-50), followed in 1998 by the identification of the minimal domain of TAT, having the amino acid sequence YGRKKRRQRRR required for protein transduction (Vives, E., P. Brodin, and B. Lebleu, A truncated HIV-1 Tat protein basic domain rapidly translocates through the plasma membrane and accumulates in the cell nucleus. J Biol Chem, 1997. 272(25): p. 16010-7). Over the past two decades, dozens of peptides were described from different origins including viral proteins, e.g. VP22 (Elliott, G. and P. O'Hare, Intercellular trafficking and protein delivery by a herpesvirus structural protein. Cell, 1997. 88(2): p. 223-33) and ZEBRA (Rothe, R., et al., Characterization of the cell-penetrating properties of the Epstein-Barr virus ZEBRA trans-activator. J Biol Chem, 2010. 285(26): p. 20224-33), or from venoms, e.g. melittin (Dempsey, C. E., The actions of melittin on membranes. Biochim Biophys Acta, 1990. 1031(2): p. 143-61), mastoporan (Konno, K., et al., Structure and biological activities of eumenine mastoparan-AF (EMP-AF), a new mast cell degranulating peptide in the venom of the solitary wasp (Anterhynchium flavomarginatum micado). Toxicon, 2000. 38(11): p. 1505-15), maurocalcin (Esteve, E., et al., Transduction of the scorpion toxin maurocalcine into cells. Evidence that the toxin crosses the plasma membrane. J Biol Chem, 2005. 280(13): p. 12833-9), crotamine (Nascimento, F. D., et al., Crotamine mediates gene delivery into cells through the binding to heparan sulfate proteoglycans. J Biol Chem, 2007. 282(29): p. 21349-60) or buforin (Kobayashi, S., et al., Membrane translocation mechanism of the antimicrobial peptide buforin 2. Biochemistry, 2004. 43(49): p. 15610-6). Synthetic CPPs were also designed including the poly-arginine (R8, R9, R10 and R12) (Futaki, S., et al., Arginine-rich peptides. An abundant source of membrane-permeable peptides having potential as carriers for intracellular protein delivery. J Biol Chem, 2001. 276(8): p. 5836-40) or transportan (Pooga, M., et al., Cell penetration by transportan. FASEB J, 1998. 12(1): p. 67-77). Any of the above described CPPs may be used as cell penetrating peptide according to the present disclosure.

The CPPs which can be used as cell penetrating peptide according to the present disclosure include but are not limited to cationic CPPs, amphipatic CPPs, and hydrophobic CPPs as well as CPPs derived from heparan-, RNA- and DNA-binding proteins, CPPs derived from signal peptides, CPPs derived from antimicrobial peptides, CPPs derived from viral proteins, CPPs derived from various natural proteins, and Designed CPPs and CPPs derived from peptide libraries (see, e.g., CPPs disclosed in Tables 1-6 of Milletti, F., 2012, Cell-penetrating peptides: classes, origin, and current landscape. Drug Discov Today 17 (15-16): 850-60).

In some embodiments, CPPs are linear peptides. In certain embodiments, CPPs used as an endosomal-lysosomal escaping moiety according to the present disclosure comprises the minimal domain of Penetratin, having the amino acid sequence RQIKIYFQNRRMKWKK. In certain embodiments, CPPs used as an endosomal-lysosomal escaping moiety according to the present disclosure comprises the minimal domain of Tat, having the amino acid sequence GRKKRRQRRRPQ. In some embodiments, CPPs used as an endosomal-lysosomal escaping moiety according to the present disclosure comprises the minimal domain of Pep-1, having the amino acid sequence KETWWETWWTEWSQP-KKKRKV. In certain embodiments, CPPs used as an endosomal-lysosomal escaping moiety according to the present disclosure comprises the minimal domain of MPG, having the amino acid sequence GALFLGFLGAAGSTMGAWSQP-KKKRKV. In certain embodiments, CPPs used as an endosomal-lysosomal escaping moiety according to the present disclosure comprises the minimal domain of Polyarginine (R₉, R₈), having the amino acid sequence RRRRRRRRR. In certain embodiments, CPPs used as an endosomal-lysosomal escaping moiety according to the present disclosure comprises the minimal domain of R₆/W₃, having the amino acid sequence RRWWRRWRR. In certain embodiments, CPPs used as an endosomal-lysosomal escaping moiety according to the present disclosure comprises the minimal domain of SAP, having the amino acid sequence (VRLPPP)₃ or (vrlppp)₃. In certain embodiments, CPPs used as an endosomal-lysosomal escaping moiety according to the present disclosure comprises the minimal domain of SAPI, having the amino acid sequence Ac-CGGW(VELPPP)₃. In certain embodiments, CPPs used as an endosomal-lysosomal escaping moiety according to the present disclosure comprises the minimal domain of CyLop-1, having the amino acid sequence CRWRWKCCKK. In certain embodiments, CPPs used as an endosomal-lysosomal escaping moiety according to the present disclosure comprises the minimal domain of gH 625, having the amino acid sequence HGLASTLTRWAHYNALIRAF. In certain embodiments, CPPs used as an endosomal-lysosomal escaping moiety according to the present disclosure comprises the minimal domain of GALA, having the amino acid sequence WEAALAEALAEALAEHLAEALAEALEALAA. In certain embodiments, CPPs used as an endosomal-lysosomal escaping moiety according to the present disclosure comprises the minimal domain of TP10, having the amino acid sequence AGYLLGKINLKALAALAKKIL. In certain embodiments, CPPs used as an endosomal-lysosomal escaping moiety according to the present disclosure comprises the minimal domain of CADY, having the amino acid sequence Ac-GLWRALWRLLRSLWRLLWRA-cysteamide. In certain embodiments, CPPs used as an endosomal-lysosomal escaping moiety according to the present disclosure comprises the minimal domain of L17E, having the amino acid sequence IWLTALKFLGKHAAKHEAKQQLSKL. In certain embodiments, CPPs used as an endosomal-lysosomal escaping moiety according to the present disclosure comprises the minimal domain of MPPs, having the amino acid sequence Mitochondria-penetrating peptides, such as FXrFXKFXrFXK. In some embodiments, CPPs used as an endosomal-lysosomal escaping moiety according to the present disclosure comprises the minimal domain of Ac-1, having the amino acid sequence Ac-(RR-Aib)₃. In certain embodiments, CPPs used as an endosomal-lysosomal escaping moiety according to the present disclosure comprises the minimal domain of Ent Ac-1, having the amino acid sequence Ac-(rr-Aib)₃. In certain embodiments, CPPs used as an endosomal-lysosomal escaping moiety according to the present disclosure comprises the minimal domain of Ac-2, having the amino acid sequence Ac-(Rr-Aib)₃. In certain embodiments, CPPs used as an endosomal-lysosomal escaping moiety according to the present disclosure comprises the minimal domain of Peptide 3, having the amino acid sequence FAM-β-Ala-(RRP^{Gu})₃G₃. In certain embodiments, CPPs used as an endosomal-lysosomal escaping moiety according to the present disclosure comprises the minimal domain of RR5-App, having the amino acid sequence RRPRRPRRPRRPGRRAPVEDLIRFYNDLQQYLNVVTRHRYC. In certain embodiments, CPPs used as an endosomal-lysosomal escaping moiety according to the present disclosure comprises the minimal domain of RR4-App, having the amino acid sequence RRPRRPRRPGRRAPVEDLIRFYNDLQQYLNVVTRHRYC. In certain embodiments, CPPs used as an endosomal-lysosomal escaping moiety according to the present disclosure comprises the minimal domain of RR3-aPP, having the amino acid sequence GPRRPRRPGRRAPVEDLIRFYNDLQQYLNVVTRHRYC. In certain embodiments, CPPs used as an endosomal-lysosomal escaping moiety according to the present disclosure comprises the minimal domain of TATp-D, having the amino acid sequence Ac-GRKKRRQRRRPPQ-K(K)-QPPRRRQRRKKRG-Ac. In certain embodiments, CPPs used as an endosomal-lysosomal escaping moiety according to the present disclosure comprises the minimal domain of R4-R4, having the amino acid sequence RRRRC-CRRRR. In certain embodiments, CPPs used as an endosomal-lysosomal escaping moiety according to the present disclosure comprises the minimal domain of R5-R5, having the amino acid sequence RRRRRC-CRRRRR.

In some embodiments, CPPs are cyclic peptides. In certain embodiments, CPPs used as an endosomal-lysosomal escaping moiety according to the present disclosure comprises the minimal domain of [WR]₄, having the amino acid sequence c[WRWRWRWR]c. In certain embodiments, CPPs used as an endosomal-lysosomal escaping moiety according to the present disclosure comprises the minimal domain of Cyclic Tat, having the amino acid sequence c[K-rRrQrRkKrG-E]c. In certain embodiments, CPPs used as an endosomal-lysosomal escaping moiety according to the present disclosure comprises the minimal domain of cFΦR₄, having the amino acid sequence c[FFRRRRQ]c. In certain embodiments, CPPs used as an endosomal-lysosomal escaping moiety according to the present disclosure comprises the minimal domain of Danamide D, having the amino acid sequence c-[I(Thz)-tBuGly-FPIP]. In certain embodiments, CPPs used as an endosomal-lysosomal escaping moiety according to the present disclosure comprises the minimal domain of Pro-(Xaa)₄-Tyr, having the amino acid sequence c[D-Pro-L-MeLeu-D-MeLeu-D/L-MeLeu-D/L-Leu-L-Tyr]c. In certain embodiments, CPPs used as an endosomal-lysosomal escaping moiety according to the present disclosure comprises the minimal domain of Cyclic sC18, having the amino acid sequence c[GLRKRLRKFRNK]c-IKEK. In some embodiments, CPPs used as an endosomal-lysosomal escaping moiety according to the present disclosure comprises the minimal domain of RRRRΦF, having the amino acid sequence

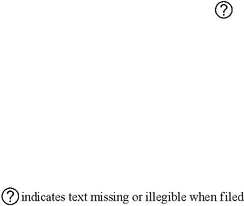

In certain embodiments, CPPs used as an endosomal-lysosomal escaping moiety according to the present disclosure comprises the minimal domain of BIM SAHB9, having the amino acid sequence IWIAQELRXIGDXFNAYYARR. In certain embodiments, CPPs used as an endosomal-lysosomal escaping moiety according to the present disclosure comprises the minimal domain of SAH-SOS1, having the amino acid sequence ZFGIYLTXILKTEEGN. In certain embodiments, CPPs used as an endosomal-lysosomal escaping moiety according to the present disclosure comprises the minimal domain of 4-R, having the amino acid sequence FITC-β-A (iso-DRRX)WRRW. In certain embodiments, CPPs used as an endosomal-lysosomal escaping moiety according to the present disclosure comprises the minimal domain of 4-W, having the amino acid sequence FITC-β-A (iso-DWWX)RWWR.

In some embodiments, CPPs are supramolecular CPPs. In some embodiments, CPPs used as an endosomal-lysosomal escaping moiety according to the present disclosure comprises the minimal domain of Sp-CC-PEG₂₀₀₀, having the amino acid sequence Sp-REGVAKALRAVANALHYNASALEEVADALQKVKM-PEG. In certain embodiments, CPPs used as an endosomal-lysosomal escaping moiety according to the present disclosure comprises the minimal domain of K₁₀(QW)₆, having the amino acid sequence KKKKKKKKKKQWQWQWQWQWQW. In certain embodiments, CPPs used as an endosomal-lysosomal escaping moiety according to the present disclosure comprises the minimal domain of YTA4, having the amino acid sequence C16-IAWVKAFIRKLRKGPLGGPLGIAGQ-RGDS. In certain embodiments, CPPs used as an endosomal-lysosomal escaping moiety according to the present disclosure comprises the minimal domain of V2, having the amino acid sequence FITC-AβGG-POGPOGPOGPOGPOGPOGPOGPOGRRRRRR. In certain embodiments, CPPs used as an endosomal-lysosomal escaping moiety according to the present disclosure comprises the minimal domain of W3, having the amino acid sequence WWW.

In some embodiments, CPPs a62eptideido mimetics. In certain embodiments, CPPs used as an endosomal-lysosomal escaping moiety according to the present disclosure comprises the minimal domain of CPPMs, which is synthetic mimics of CPPs. In certain embodiments, CPPs used as an endosomal-lysosomal escaping moiety according to the present disclosure comprises the minimal domain of Peptoids (CPPos), which is peptidomimetic molecules. In certain embodiments, CPPs used as an endosomal-lysosomal escaping moiety according to the present disclosure comprises the minimal domain of CPDs, which is cell-penetrating disulfides.

Exemplary CPPs useful in the present disclosure are disclosed in Daniela Kalafatovic et al., “Cell-Penetrating Peptides: Design Strategies beyond Primary Structure and Amphipathicity”, Molecules 2017, 22, 1929.

### Endosomal Escape Domain (EED)

According to the embodiments described herein, endosomal-lysosomal escape moiety described herein may comprise one or more endosomal escape domain or lysosomal escape domain, which are signals that cause the internalized biotag to escape from endocytotic and lysosomal pathways, resulting in permanently tagging the target cancer cell with the biotag, acting as a reporter. EEDs address the critical rate-limiting step of endosomal escape in delivery of macromolecular biologic peptide, protein and siRNA therapeutics into cells. In some embodiments, the endosomal escape domain may include, but is not limited to GG, GFFG, GFWFG, GWGGWG, GWG, GFWG, GWWG, and GWWG. In some embodiments, an endosomal-lysosomal escape moiety has a nuclear localization sequence, which is the signal guiding the entry of the biotag into the cell nucleus.

In one embodiment, the endosomal escape domain containing two aromatic indole rings or one indole ring and two aromatic phenyl groups at a fixed distance of six polyethylene glycol (PEG) units from the TAT-PTD-cargo. In some embodiments, the EED comprises 1 to 8 amino acids and comprises from 2-5 aromatic rings. In certain embodiments, the EEDs further comprises a hydrophilic polymer spacer between the PTD and the aromatic-rich peptide domain. In certain embodiments, the hydrophilic polymer spacer comprises polyethylene glycol having 1-18 (e.g., 1, 2, 3, 4, 5, 6, 7, 8, 9, 10, 11, 12, 13, 14, 15, 16, 17 or 18) PEG moieties. In some embodiments, an EED comprises the aromatic-rich peptide domain and a PEG linker. In certain embodiments, the EED comprises 1 to 8 amino acids comprising from 3-5 aromatic groups and a spacer of 2-18 PEG moieties. In yet another embodiment, the EED comprises 4 aromatic groups. In some embodiments, the EED does not comprise more than 3 phenylalanines in series (e.g., no more than 2 adjacent phenylalanines). In some embodiments, the aromatic-rich peptide domain comprises a peptide selected from the group consisting of GFFG, GWG, GFWG, GFWFG, GWWG and GWGGWG.

Exemplary EDDs useful in the present disclosure are disclosed in Peter Lonn et al., “Enhancing Endosomal Escape for Intracellular Delivery of Macromolecular Biologic Therapeutics”, Sci. Rep. 2016, 6, 32301.

### Cationic Polymer

Polymers have emerged as a viable alternative endosomal-lysosomal escape moiety because of their excellent molecular flexibility allows for complex modifications and incorporation of novel chemistries. The transfection efficiency of the cationic polymers is influenced by their molecular weight. In some embodiments, the cation polymers according to the present disclosure can be selected from any suitable cationic polymers which have been widely studied as gene delivery candidates due to their ability to condense DNA, and promote DNA stability and transmembrane delivery. In some embodiments, the polymers have high molecular weight, e.g., greater than 20 kD.

In some embodiments, the cationic polymers according to the present disclosure is selected from poly(L-lysine) (PLL), poly(L-arginine) (PLA), polyethyleneimine (PEI), diethylaminoethyl-dextran (DEAE-DEX), and polyamidoamine (PAMAM) dendrimers. In certain embodiments, the cationic polymer is a polyamine, such as a polyethyleneimine, which condenses DNA into small narrowly distributed positively charged spherical complexes and can transfect cells in vitro and in vivo. PEI is similar to other cationic polymers in that the transfection activity of PEI increases with increasing polymer/DNA ratios. Commercial branched PEI is composed of 25% primary amines, 50% secondary amines and 25% tertiary amines.

### Linker-Polymer-Oligonucleotide Compound

In one aspect, there is provided a polymeric scaffold of Formula (I) or Formula (II) useful to conjugate with a targeting moiety:

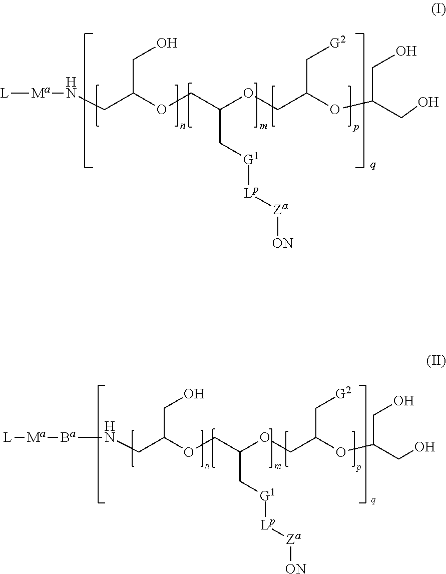

wherein:
the polymeric scaffold comprises linear polyglycerol;
L is a linking moiety comprising a functional group Wₚ that is capable of forming a covalent bond with the targeting moiety;
M^{a} is a stretcher connecting L to B^{a} or —NH— moiety;
B^{a} is a branching moiety comprising a functional group W_{M} connecting to —NH-moiety;
each G¹ is independently a functional group connecting L^{p} to the linear polyglycerol;
each L^{P} is independently a drug release mechanism between Z^{a} and G¹;
each Z^{a} is independently an endosomal-lysosomal escaping moiety or a conjugation moiety;
each ON is independently an oligonucleotide;
each G² is independently a functional group capable of converting into a charged state;
n is an integer from 0 to 1000;
m is an integer from 0 to 1000;
p is an integer from 0 to 1000; and
q is an integer from 0 to 1000.

In some embodiments, n is an integer from 0 to 1000, from 0 to 500, from 0 to 400, from 0 to 300, from 0 to 200, from 0 to 100, from 0 to 90, from 0 to 80, from 0 to 70, from 0 to 60, from 0 to 50, from 0 to 40, from 0 to 30, from 0 to 20, from 0 to 10, from 0 to 9, from 0 to 8, from 0 to 7, from 0 to 6, from 0 to 5, from 0 to 4, from 0 to 3, or from 0 to 2.

In some embodiments, m is an integer from 0 to 1000, from 0 to 500, from 0 to 400, from 0 to 300, from 0 to 200, from 0 to 100, from 0 to 90, from 0 to 80, from 0 to 70, from 0 to 60, from 0 to 50, from 0 to 40, from 0 to 30, from 0 to 20, from 1 to 10, from 1 to 9, from 1 to 8, from 1 to 7, from 1 to 6, from 1 to 5, from 1 to 4, from 1 to 3, or from 1 to 2.

In some embodiments, p is an integer from 0 to 1000, from 0 to 500, from 0 to 400, from 0 to 300, from 0 to 200, from 0 to 100, from 0 to 90, from 0 to 80, from 0 to 70, from 0 to 60, from 0 to 50, from 0 to 40, from 0 to 30, from 0 to 20, from 1 to 10, from 1 to 9, from 1 to 8, from 1 to 7, from 1 to 6, from 1 to 5, from 1 to 4, from 1 to 3, or from 1 to 2.

In some embodiments, q is an integer from 0 to 1000, from 0 to 500, from 0 to 400, from 0 to 300, from 0 to 200, from 0 to 100, from 0 to 90, from 0 to 80, from 0 to 70, from 0 to 60, from 0 to 50, from 0 to 40, from 0 to 30, from 0 to 20, from 1 to 10, from 1 to 9, from 1 to 8, from 1 to 7, from 1 to 6, from 1 to 5, from 1 to 4, from 1 to 3, or from 1 to 2.

In some embodiments, Wₚ is capable of reacting with a functional group on the targeting moiety with a click reaction.

In certain embodiments, Wₚ is selected from the group consisting of:

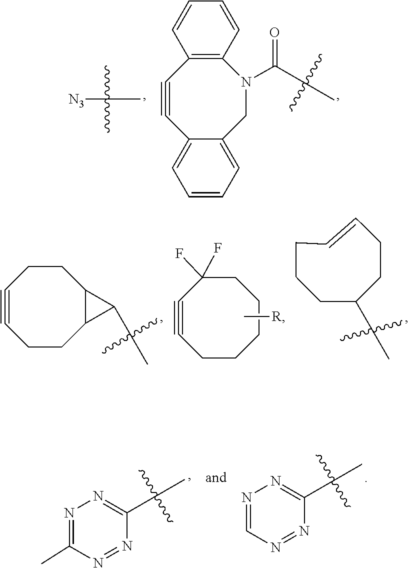

In some embodiments, Wₚ is capable of reacting with amino acids on the targeting moiety.

In certain embodiments, Wₚ is capable of reacting with amino acids on the targeting moiety, and the amino acids are natural amino acids, non-natural amino acids or combination thereof. In certain embodiments, the natural amino acid may comprise cysteine, lysine, tyrosine, aspartic acid and glutamic acid.

In some embodiments, Wₚ is capable of reacting with one or more cysteines on the targeting moiety.

In certain embodiments, Wₚ is capable of reacting with one or more cysteines on the targeting moiety and each Wₚ is selected from the group consisting of:

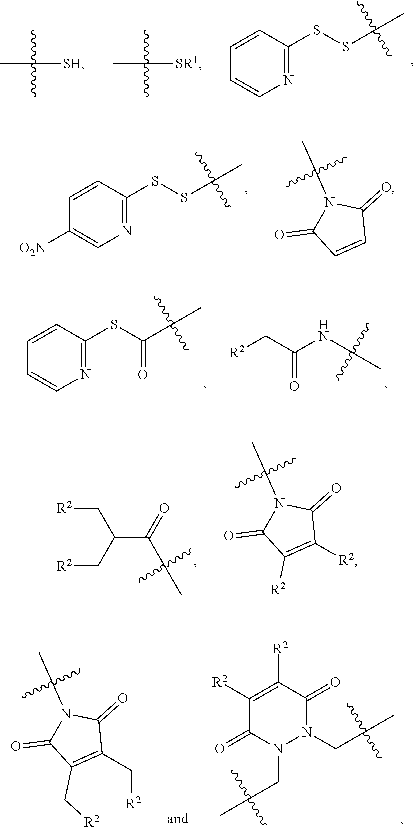

wherein R¹ is a sulfur protecting group, and each R² is independently a leaving group.

In certain embodiments, each R² is independently selected from halo or R^{2a}C(O)O—, in which R^{2a} is hydrogen, aliphatic, heteroaliphatic, cycloalkyl, heterocycloalkyl, aryl or heteroaryl.

In some embodiments, Wₚ is capable of reacting with one or more lysines on the targeting moiety.

In certain embodiments, Wₚ is capable of reacting with one or more lysines on the targeting moiety, and each Wₚ is independently selected from the group consisting of:

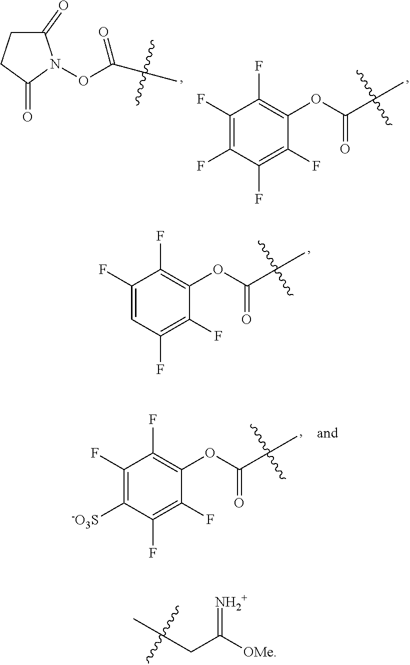

In some embodiments, Wₚ is capable of reacting with one or more non-natural amino acids on the targeting moiety.

In certain embodiments, Wₚ is capable of reacting with one or more non-natural amino acids on the targeting moiety and each Wₚ is independently selected from:

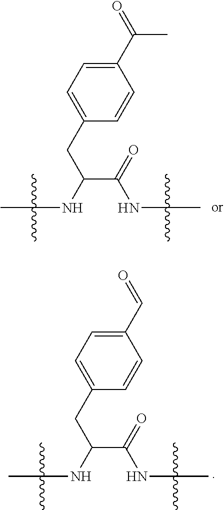 [SMILES: *NC(=O)C(Cc1ccc(C(C)=O)cc1)N* ; *NC(=O)C(Cc1ccc(C=O)cc1)N*]

In some embodiments, L is

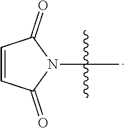 [SMILES: *N1C(=O)C=CC1=O]

In some embodiments, M^{a} is selected from the group consisting of

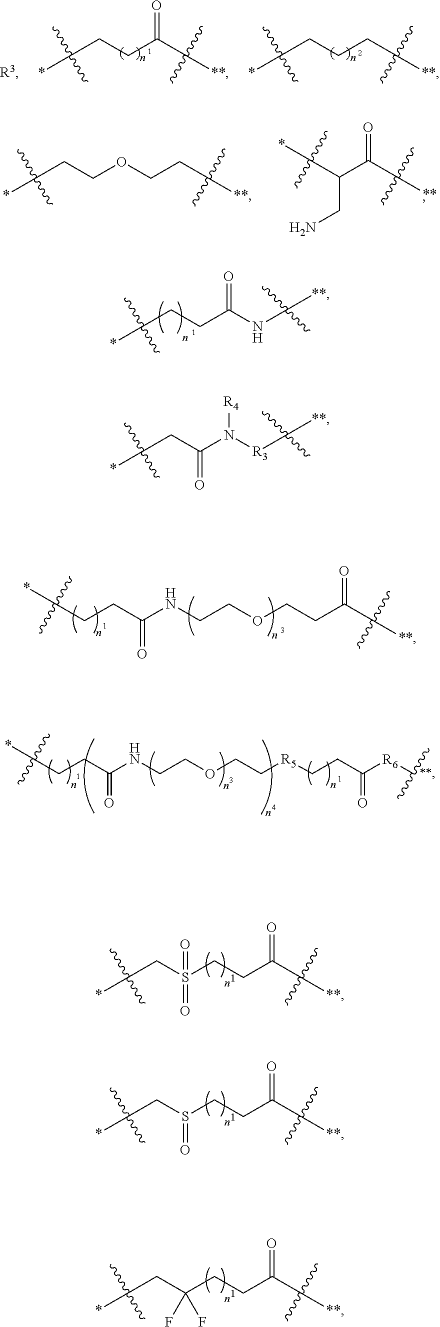

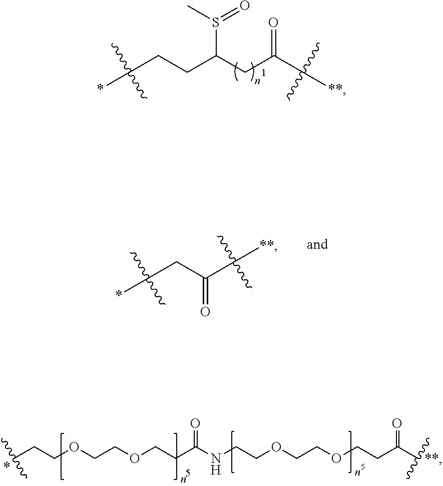

wherein
* is the site covalently attached to L, ** is the site covalently attached to B^{a} or —NH— moiety;
R³ is C₁₋₁₀ alkyl, C₁₋₁₀ heteroalkyl, C₃₋₈ cycloalkyl, —O—(C₁₋₈ alkyl)-, aryl, —C₁₋₁₀ alkyl-aryl-, -aryl-C₁₋₁₀ alkyl-, —C₁₋₁₀ alkyl-(C₃₋₈ cycloalkyl)-, —(C₃₋₈ cycloalkyl-C₁₋₁₀ alkyl)-, 4- to 14-membered heterocycloalkyl, —C₁₋₁₀ alkyl-(4- to 14-membered heterocycloalkyl)-, -(4- to 14-membered heterocycloalkyl)-C₁₋₁₀ alkyl-, —C₁₋₁₀ alkyl-C(═O)—, —C₁₋₁₀ heteroalkyl-C(═O)—, —C₃₋₈ cycloalkyl-C(═O)—, —O—(C₁₋₈alkyl)-C(═O)—, -aryl-C(═O)—, —C₁₋₁₀ alkyl-aryl-C(═O)—, -aryl-C₁₋₁₀ alkyl-C(═O)—, —C₁₋₁₀ alkyl-(C₃₋₈ cycloalkyl)-C(═O)—, —(C₃₋₈ cycloalkyl)-C₁₋₁₀ alkyl-C(═O)—, -4- to 14-membered heterocycloalkyl-C═(O)—, —C₁₋₁₀ alkyl-(4- to 14-membered heterocycloalkyl)-C(═O)—, -(4- to 14-membered heterocycloalkyl)-C₁₋₁₀ alkyl-C(═O)—, —C₁₋₁₀ alkyl-NH—, —C₁₋₁₀ heteroalkyl-NH—, —C₃₋₈ cycloalkyl-NH—, —O—(C₁₋₈ alkyl)-NH—, -aryl-NH—, —C₁₋₁₀ alkyl-aryl-NH—, -aryl-C₁₋₁₀ alkyl-NH—, —C₁₋₁₀ alkyl-(C₃₋₈ cycloalkyl)-NH—, —(C₃₋₈ cycloalkyl)-C₁₋₁₀ alkyl-NH—, -4- to 14-membered heterocycloalkyl-NH—, —C₁₋₁₀ alkyl-(4- to 14-membered heterocycloalkyl)-NH—, -(4- to 14-membered heterocycloalkyl)-C₁₋₁₀ alkyl-NH—, —C₁₋₁₀ alkyl-S—, —C₁₋₁₀ heteroalkyl-S—, —C₃₋₈ cycloalkyl-S—, —O—C₁₋₈ alkyl-S—, -aryl-S—, —C₁₋₁₀ alkyl-aryl-S—, -aryl-C₁₋₁₀ alkyl-S—, —C₁₋₁₀ alkyl-(C₃₋₈cycloalkyl)-S—, —(C₃₋₈ cycloalkyl)-C₁₋₁₀ alkyl-S—, -4- to 14-membered heterocycloalkyl-S—, —C₁₋₁₀ alkyl-(4- to 14-membered heterocycloalkyl)-S—, -r-(4- to 14-membered heterocycloalkyl)-C₁₋₁₀ alkyl-S—;
each R⁴ independently is hydrogen, C₁₋₆ alkyl, C₆₋₁₀ aryl, C₃₋₈ cycloalkyl, —COOH or —COO—C₁₋₆ alkyl;
R⁵ is —C(O)—NR^{5a} or —NR^{5a}—C(O)—;
R^{5a} is hydrogen, C₁₋₆ alkyl, C₆₋₁₀ aryl, C₃₋₈ cycloalkyl, —COOH or —COO—C₁₋₆ alkyl;
R⁶ is a bond or —NR^{6a}—(CR^{6b}R^{6c})—C(O)—;
R^{6a} is hydrogen, C₁₋₆ alkyl, C₆₋₁₀ aryl, C₃₋₈ cycloalkyl, —COOH or —COO—C₁₋₆ alkyl;
each R^{6b} and R^{6c} independently is hydrogen, C₁₋₆ alkyl, C₆₋₁₀ aryl, hydroxylated C₆₋₁₀ aryl, polyhydroxylated C₆₋₁₀ aryl, 5- to 12-membered heterocycloalkyl, C₃₋₈ cycloalkyl, hydroxylated C₃₋₈ cycloalkyl, polyhydroxylated C₃₋₈ cycloalkyl or a side chain of a natural or unnatural amino acid;
each n¹ independently is an integer from 0 to 6;
n² is an integer from 0 to 8;
each n³ independently is an integer from 1 to 6;
n⁴ is an integer from 1 to 4; and
n⁵ is an integer from 1 to 4.

In certain embodiments, M^{a} is selected from the group consisting of:

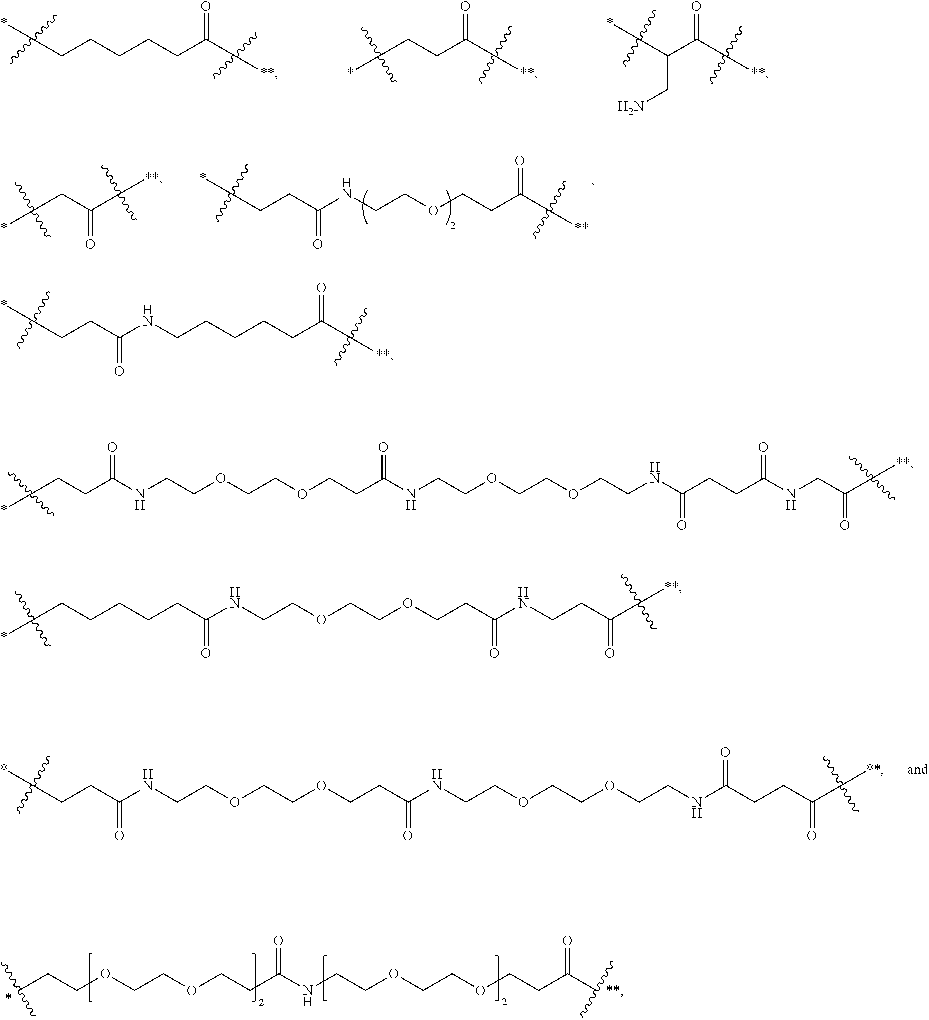

wherein * is the site covalently attached to L, ** is the site covalently attached to Bm or —NH— moiety.

In some embodiments, G¹ is selected from the group consisting of:

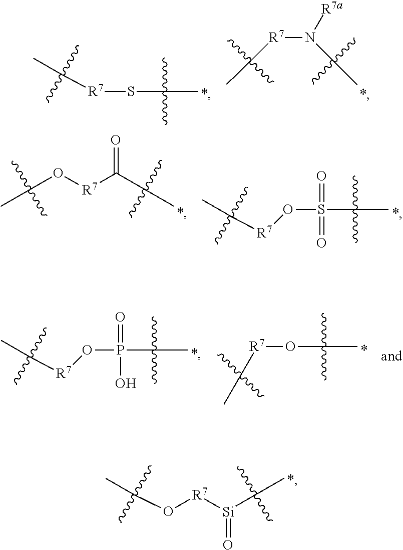

wherein * is the site covalently attached to L^{P}, each R⁷ is independently selected from a direct bond, alkyl, cycloalkyl, heterocycloalkyl, aryl, or heteroaryl, and R^{7a} is selected from hydrogen, an aliphatic, heteroaliphatic, cycloalkyl, or heterocycloalkyl moiety.

In some embodiments, each L^{p} independently comprises a labile structure.

In certain embodiments, the labile structure is selected from redox labile structures, hydrolytically labile structures or enzymatic labile structures.

In certain embodiments, the labile structure is a redox labile structure.

In certain embodiments, the labile structure is a redox labile structure having a structure of

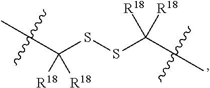

wherein each of R¹⁸ is independently selected from hydrogen, aliphatic, heteroaliphatic, cycloalkyl or heterocycloalkyl.

In certain embodiments, the labile structure is a hydrolytically labile structure is selected from the group consisting of:

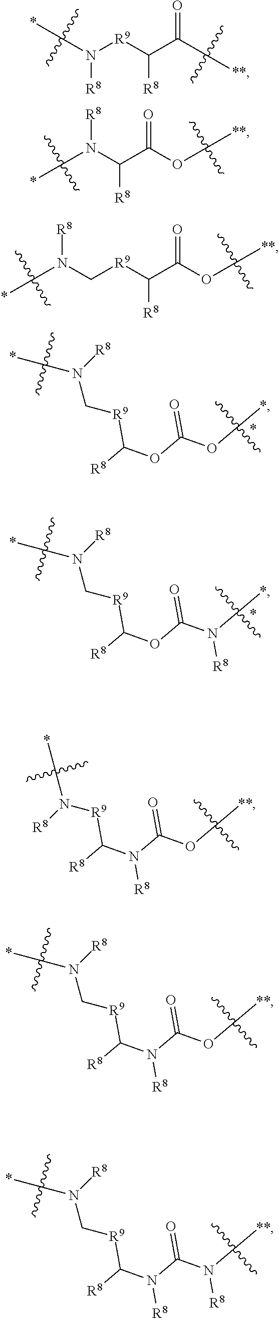

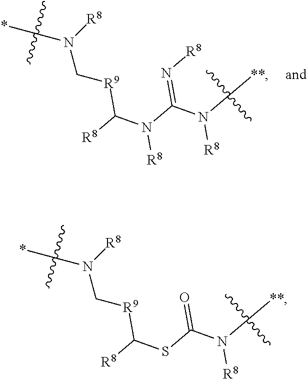

wherein * is the site covalently attached to G¹, ** is the site covalently attached to Z^{a}, R⁸ is selected from hydrogen, alkyl or aryl, R⁹ is selected from aliphatic, heteroaliphatic, cycloalkyl, heterocycloalkyl, aryl, or heteroaryl.

In certain embodiments, G¹ is

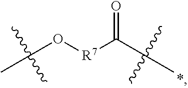

wherein * is the site covalently attached to L^{P}, and R⁷ is alkyl (such as C₁₋₆ alkyl).

In some embodiments, the labile structure is an enzymatic labile structure.

In certain embodiments, the labile structure is an enzymatic labile structure liable to enzymes selected from Cathepsin B, phosphatase, sulfatase, or glucuronidase.

In some embodiments, G¹ is

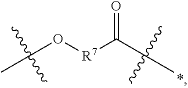

wherein * is the site covalently attached to L^{P}, and R⁷ is alkyl (such as C₁₋₆ alkyl).

In certain embodiments, the enzymatic labile structure is liable to cathepsin B and is selected from —Z— or

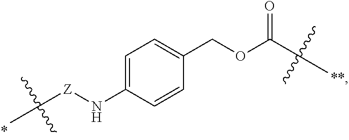

wherein * is the site covalently attached to G¹, ** is the site covalently attached to Z^{a}, and Z is a substrate for cathepsin B comprising 2 to 4 amino acids.

In certain embodiments, the enzymatic labile structure is liable to glucuronidase and is

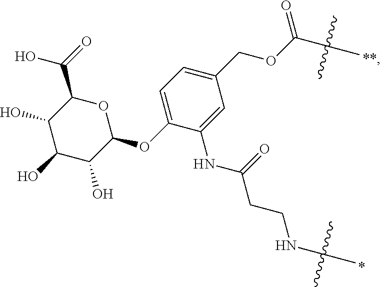 [SMILES: *NCCC(=O)Nc1cc(COC(*)=O)ccc1O[C@@H]1O[C@H](C(=O)O)[C@@H](O)[C@H](O)[C@H]1O]

wherein * is the site covalently attached to G¹, ** is the site covalently attached to Z^{a}.

In some embodiments, G¹ is

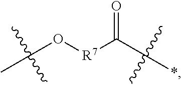

wherein * is the site covalently attached to L^{P}, and R⁷ is alkyl (such as C₁₋₆ alkyl).

In certain embodiments, the enzymatic labile structure is liable to phosphatase and is selected from

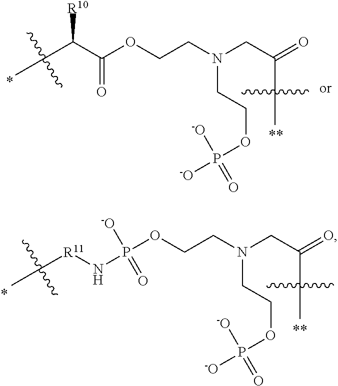

wherein * is the site covalently attached to G¹, ** is the site covalently attached to Z^{a}, and each of R¹⁰ and R¹¹ is independently hydrogen, aliphatic, heteroaliphatic, cycloalkyl, or heterocycloalkyl moiety.

In certain embodiments, G¹ is

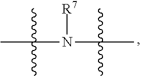

wherein R⁷ is selected from hydrogen, an aliphatic, heteroaliphatic, cycloalkyl, heterocycloalkyl, aryl or heteroaryl. In certain embodiments, R⁷ is selected from hydrogen or alkyl (such as C₁₋₆ alkyl).

In certain embodiments, the enzymatic labile structures are liable to sulfatase and is

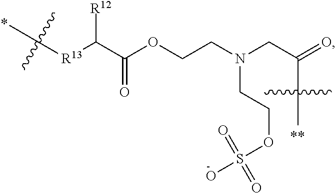

wherein * is the site covalently attached to G¹, ** is the site covalently attached to Z^{a}, and each of R¹² and R¹³ is independently hydrogen, —NH—, aliphatic, heteroaliphatic, cycloalkyl, or heterocycloalkyl moiety.

In some embodiments, G¹ is

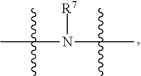

wherein R⁷ is selected from hydrogen, an aliphatic, heteroaliphatic, cycloalkyl, heterocycloalkyl, aryl or heteroaryl. In certain embodiments, R⁷ is selected from hydrogen or alkyl (such as C₁₋₆ alkyl).

In some embodiments, Z^{a} is a cell penetration peptide.

In certain embodiments, Z^{a} is selected from the group consisting of Tat, Penetratin, Pep-1, MPG, Polyarginine (R9, R8), R6/W3, SAP, I(E), CyLop-1, gH 625, GALA, TP10, CADY, L17E, MPPs, Ac-1, Ent Ac-1, Ac-2, Peptide 3, RR5-App, RR4-App, RR3-aPP, TATp-D, R4-R4, R5-R5, [WR]₄, Cyclic Tat, cFΦR₄, Danamide D, Pro-(Xaa)₄-Tyr, Cyclic sC18, RRRRqF, BIM SAHB9, SAH-SOS1, 4-R, 4-W, and Sp-CC-PEG₂₀₀₀.

In some embodiments, Z^{a} is an endosomal escape domain.

In certain embodiments, Z^{a} is selected from the group consisting of GG, GFFG, GFWFG, GWGGWG, GWG, GFWG, GWWG, and GWWG.

In some embodiments, Z^{a} is a cationic polymer.

In certain embodiments, Z^{a} is selected from PEI, PLL, PLA, DEAE-DEX or PAMAM. In certain embodiments, Z^{a} is PEI.

In some embodiments, Z^{a} is a conjugation moiety.

In certain embodiments, Z^{a} is **—NH—(CH₂)ₙ₇—* or **—S—(CH₂)ₙ₇—*, wherein n7 is an integer from 1 to 6, * is the site covalently attached to ON, and ** is the site covalently attached to L^{p}.

In certain embodiments, Z^{a} is —NH—(CH₂)₆—.

In some embodiments, -G-L^{p}-Z^{a}— is

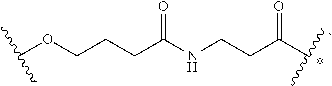 [SMILES: *OCCCC(=O)NCCC(*)=O]

wherein * is the site covalently attached to ON.

In some embodiments, n is an integer from 1 to 100; m is an integer from 1 to 100; and p is an integer from 1 to 50.

In some embodiments, ON is selected from the group consisting of antisense oligonucleotides, mRNA, siRNA, saRNA, miRNA, shRNA and CRISPER/Cas9 complex.

In certain embodiments, ON silences the TGFβ.

In certain embodiments, ON is a double strand siRNA.

In certain embodiments, sense strand sequence of the siRNA is shown as SEQ ID NO: 203, and antisense strand sequence of the siRNA is shown as SEQ ID NO: 204.

In some embodiments, ON is a double strand siRNA, and Z^{a} is connected to sense strand or antisense strand of ON at 5′ or 3′ end. In some embodiments, ON is a double strand siRNA, Z^{a} is connected to sense strand of ON at 5′ end. In some embodiments, ON is a double strand siRNA, Z^{a} is connected to sense strand of ON at 3′ end.

In certain embodiments, G² is selected from the group consisting of:

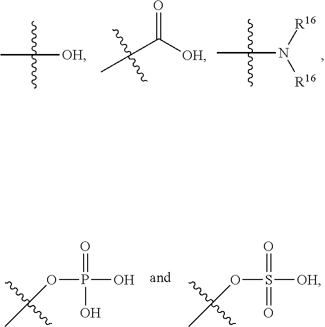

wherein each R¹⁶ is independently hydrogen, aliphatic, heteroaliphatic, cycloalkyl, heterocycloalkyl, aryl or heteroaryl. In certain embodiments, each R¹⁶ is independently hydrogen or alkyl. In certain embodiments, each R¹⁶ is independently hydrogen or C₁₋₆ alkyl.

In some embodiments, B^{a} is

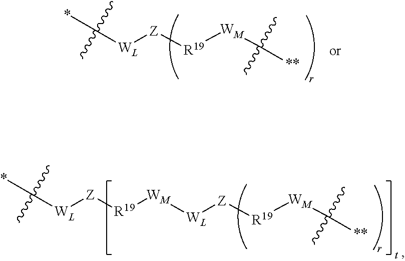

wherein W_{L} is a functional group connected to stretcher M^{a};
each Z is independently a branching point;
each R¹⁹ is independently a linker connecting Z to W_{M};
each W_{M} is independently a functional group connected to —NH—;
r is an integer from 1-3;
t is an integer from 1-3;
wherein * is the site covalently attached to M^{a}, ** is the site covalently attached to —NH— moiety.

In some embodiments, the —NH— moiety is

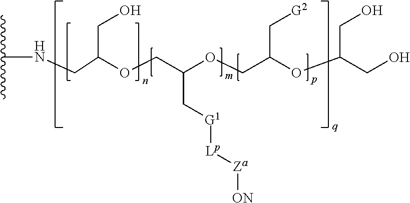

or —NH—(CH₂)ₙ₆—COOH, wherein G¹, L^{p}, Z^{a}, G², ON, n, m, p and q are as defined supra, and n⁶ is an integer from 1 to 6.

In certain embodiments, W_{L} is selected from a group consisting of:

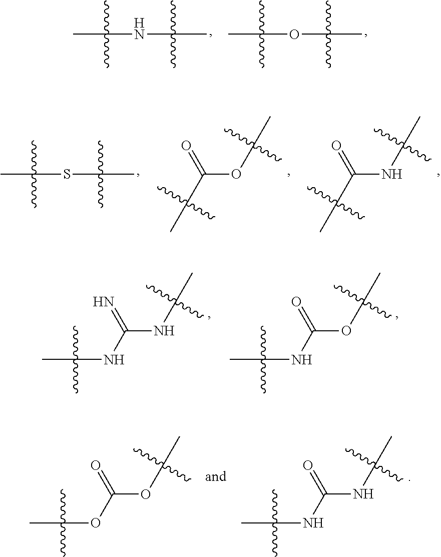

In certain embodiments, Z is —CH₍₃₋ᵣ₎—, -SiH₍₃₋ᵣ₎— or —NH₍₂₋ᵣ₎—.

In certain embodiments, R¹⁹ is selected from a group consisting of an aliphatic, heteroaliphatic, cycloalkyl, and heterocycloalkyl. In certain embodiments, R¹⁹ is alkyl or heteroalkyl.

In some embodiments, B^{a} is

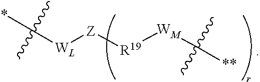

In certain embodiments, B^{a} is selected from the group consisting of:

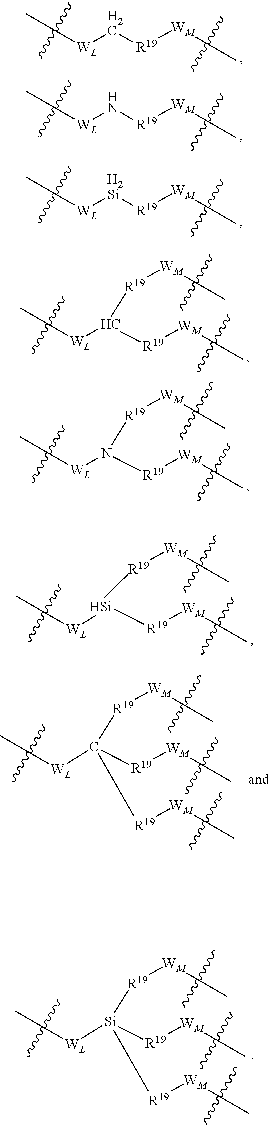

In some embodiments, B^{a} is

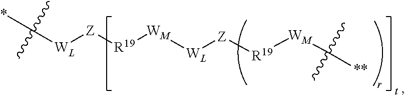

In certain embodiments, B^{a} is selected from the group consisting of:

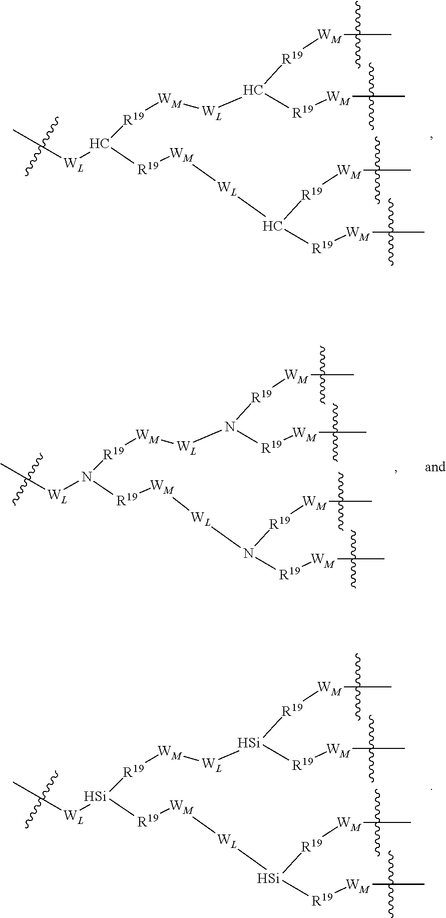

In some embodiments, B^{a} is

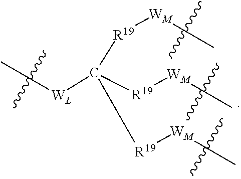

In some embodiments, two of the W_{M} are connected to —NH—(CH₂)ₙ₆—COOH, and one of the W_{M} is connected to

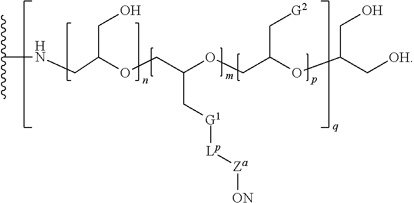

In some embodiments, three W_{M} are connected to

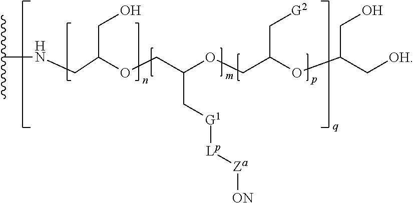

In certain embodiments, W_{L} is

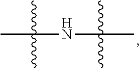 [SMILES: *N*]

and —R⁹—W_{M}— is

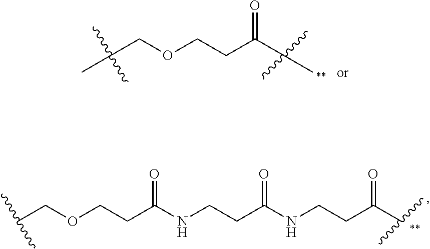 [SMILES: *COCCC(*)=O ; *COCCC(=O)NCCC(=O)NCCC(*)=O]

wherein ** is the site covalently attached to W_{L} or —NH— moiety.

In certain embodiments, B^{a} is

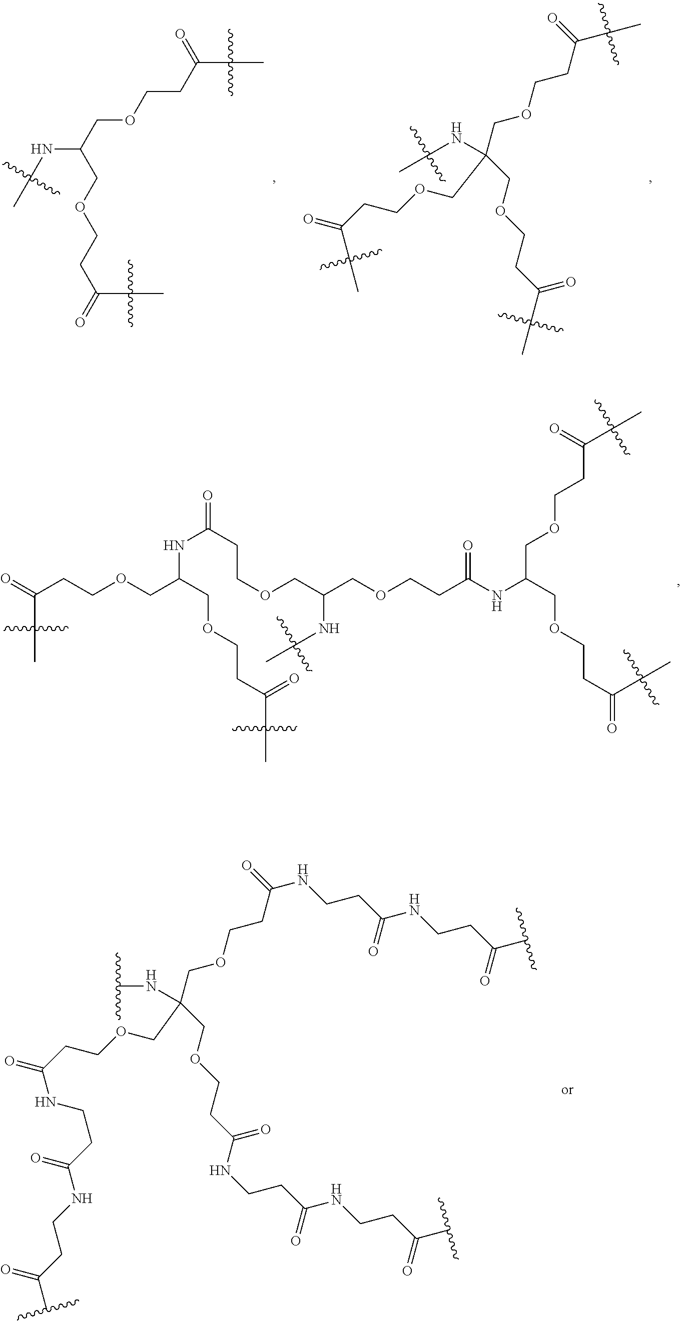 [SMILES: *NC(COCCC(*)=O)COCCC(*)=O ; *NC(COCCC(*)=O)(COCCC(*)=O)COCCC(*)=O ; *NC(COCCC(=O)NC(COCCC(*)=O)COCCC(*)=O)COCCC(=O)NC(COCCC(*)=O)COCCC(*)=O ; *NC(COCCC(=O)NCCC(=O)NCCC(*)=O)(COCCC(=O)NCCC(=O)NCCC(*)=O)COCCC(=O)NCCC(=O)NCCC(*)=O]

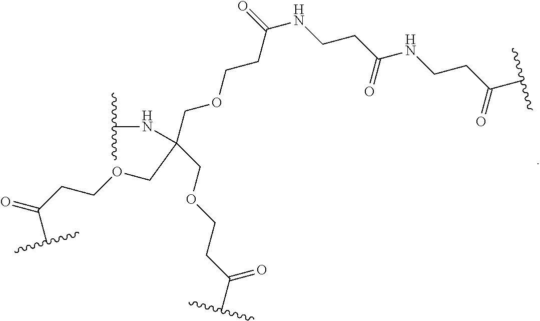 [SMILES: *NC(COCCC(*)=O)(COCCC(*)=O)COCCC(=O)NCCC(=O)NCCC(*)=O]

In certain embodiments, the polymeric scaffold provided herein is selected from the group consisting of

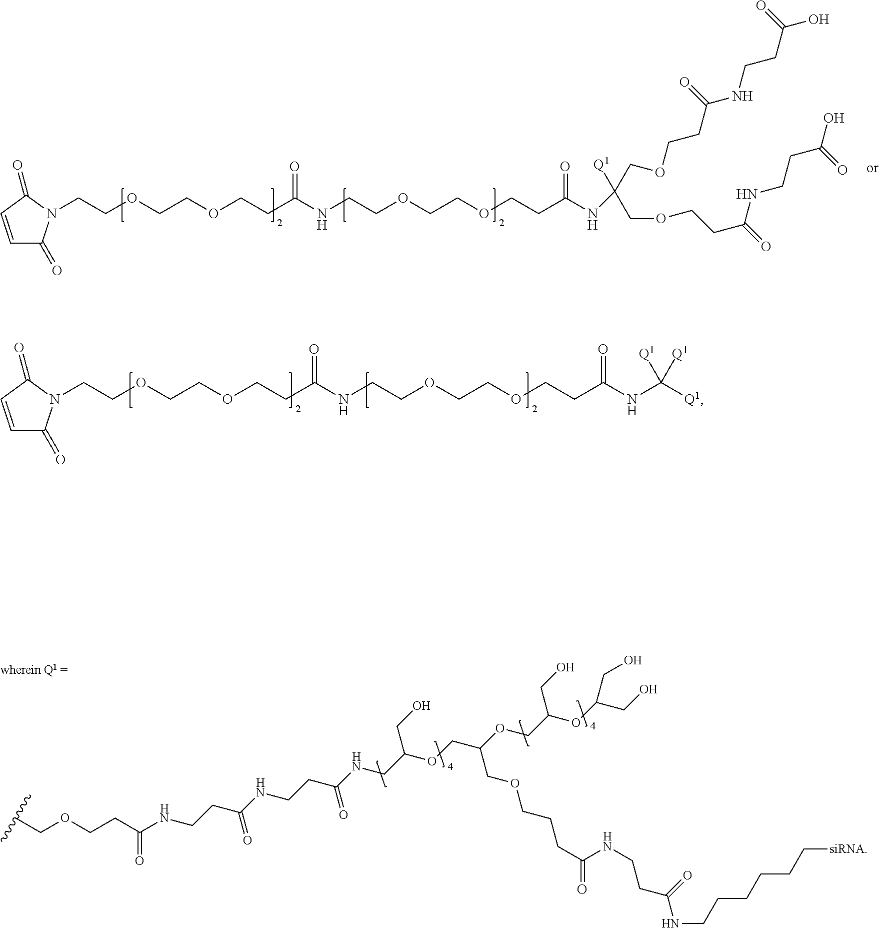

### Targeting Moiety

The targeting moiety directs the linker-polymer-oligonucleotide conjugates to specific tissues, cells, or locations in a cell. The targeting moiety can direct the modified polymer in culture or in a whole organism, or both. In each case, the targeting moiety can bind to a ligand that is present on the cell surface of the targeted cell(s) with an effective specificity, affinity and avidity. In some embodiments, the targeting moiety targets the modified polymer to tissues other than the liver. In other embodiments, the targeting moiety targets the modified polymer to a specific tissue such as the liver, kidney, lung or pancreas. The targeting moiety can target the modified polymer to a target cell such as a cancer cell, such as a receptor expressed on a cell such as a cancer cell, a matrix tissue, or a protein associated with cancer such as tumor antigen. Alternatively, cells comprising the tumor vasculature may be targeted. The targeting moiety can direct the modified polymer to specific types of cells such as specific targeting to hepatocytes in the liver as opposed to Kupffer cells. In other cases, the targeting moiety can direct the modified polymer to cells of the reticular endothelial or lymphatic system, or to professional phagocytic cells such as macrophages or eosinophils.

In still other embodiments, the targeting moiety can target the modified polymer to a location within the cell, such as the nucleus, the cytoplasm, or the endosome, for example. In certain embodiments, the targeting moiety can enhance cellular binding to receptors, or cytoplasmic transport to the nucleus and nuclear entry or release from endosomes or other intracellular vesicles.

In some embodiments, the targeting moiety includes antibodies, proteins and peptides or peptide mimics.

In some embodiments, the targeting moiety comprises natural amino acids that are capable of reacting with a functional group in the linking moiety of the linker-polymer-oligonucleotide conjugate to form a covalent bond. In certain embodiments, the natural amino acid includes cysteine, lysine, tyrosine, aspartic acid and glutamic acid.

In certain embodiments, the targeting moiety comprises cysteine and the targeting moiety is conjugated to the linker-polymer-oligonucleotide conjugate by a covalent bond via the sulfhydryl group and a functional group of the linking moiety in the linker-polymer-oligonucleotide conjugate.

In certain embodiments, the targeting moiety comprises lysine and the targeting moiety is conjugated to the linker-polymer-oligonucleotide conjugate by a covalent bond via the amino group and a functional group of the linking moiety in the linker-polymer-oligonucleotide conjugate.

In some embodiments, the targeting moiety may comprise non-natural amino acids that are capable of reacting with a functional group in the linking moiety of the linker-polymer-oligonucleotide conjugate to form a covalent bond. In certain embodiments, the targeting moiety is conjugated to the linker-polymer-oligonucleotide conjugate by a covalent bond via the amino group and a functional group of the linking moiety in the linker-polymer-oligonucleotide conjugate.

In some embodiments, the targeting moiety may comprise functional groups that are capable of reacting with a functional group in the linking moiety of the linker-polymer-oligonucleotide conjugate via click reaction to form a covalent bond.

In some embodiments, the targeting moiety can be antibodies or antibodies derived from Fab, Fab2, scFv or camel antibody heavy-chain fragments specific to the cell surface markers, including but not limited to, 5T4, AOC3, ALK, AXL, C242, CA-125, CCL11, CCR 5, CD2, CD3, CD4, CD5, CD15, CA15-3, CD18, CD19, CA19-9, CD20, CD22, CD23, CD25, CD28, CD30, CD31, CD33, CD37, CD38, CD40, CD41, CD44, CD44 v6, CD51, CD52, CD54, CD56, CD62E, CD62P, CD62L, CD70, CD74, CD79-B, CD80, CD125, CD138, CD141, CD147, CD152, CD154, CD326, CEA, clumping factor, CTLA-4, CXCR2, EGFR (HER1), ErbB2, ErbB3, EpCAM, EPHA2, EPHB2, EPHB4, FGFR (i.e. FGFR1, FGFR2, FGFR3, FGFR4), FLT3, folate receptor, FAP, GD2, GD3, GPNMB, HGF, HMI.24, ICAM, ICOS-L, IGF-1 receptor, VEGFR1, EphA2, TRPV1, CFTR, gpNMB, CA9, Cripto, c-KIT, c-MET, ACE, APP, adrenergic receptor-beta2, Claudine 3, Mesothelin, MUC1, NaPi2b, NOTCH1, NOTCH2, NOTCH3, NOTCH4, RON, ROR1, PD-L1, PD-L2, B7-H3, B7-B4, IL-2 receptor, IL-4 receptor, IL-13 receptor, Trop-2, integrins (including α₄, αᵥβ₃, αᵥβ₅, αᵥβ₆, α₁β₄, α₄β₁, α₄β₇, α₅β₁, α₆β₄, α_{IIb}β₃ integrins), IFN-α, IFN-γ, IgE, IgE, IGF-1 receptor, IL-1, IL-12, IL-23, IL-13, IL-22, IL-4, IL-5, IL-6, interferon receptor, ITGB2 (CD18), LFA-1 (CD11a), L-selectin (CD62L), mucin, MUC1, myostatin, NCA-90, NGF, PDGFRα, phosphatidylserine, prostatic carcinoma cell, Pseudomonas aeruginosa, rabies, RANKL, respiratory syncytial virus, Rhesus factor, SLAMF7, sphingosine-1-phosphate, TAG-72, T-cell receptor, tenascin C, TGF-1, TGF-β2, TGF-β, TNF-α, TRAIL-R1, TRAIL-R2, tumor antigen CTAA16.88, VEGF-A, VEGFR2, vimentin, and the like.

In certain embodiments, the antibodies or antibody derived from Fab, Fab2, scFv or camel antibody heavy-chain fragments specific to the cell surface markers include CA-125, C242, CD3, CD19, CD22, CD25, CD30, CD31, CD33, CD37, CD40, CD44, CD51, CD54, CD56, CD62E, CD62P, CD62L, CD70, CD138, CD141, CD326, CEA, CTLA-4, EGFR (HER1), ErbB2, ErbB3, FAP, folate receptor, IGF-1 receptor, GD3, GPNMB, HGF, VEGF-A, VEGFR2, VEGFR1, EphA2, EpCAM, 5T4, TAG-72, tenascin C, TRPV1, CFTR, gpNMB, CA9, Cripto, ACE, APP, PDGFR α, phosphatidylserine, prostatic carcinoma cells, adrenergic receptor-beta2, Claudine 3, mucin, MUC1, Mesothelin, IL-2 receptor, IL-4 receptor, IL-13 receptor and integrins (including αᵥβ₃, αᵥβ₅, αᵥβ₆, α₁β₄, α₄β₁, α₅β₁, α₆β₄ integrins), tenascin C, TRAIL-R2 and vimentin.

In certain embodiments, the targeting moiety is anti-αvβ6 antibody, or antigen-binding fragment thereof. Examples of anti-αvβ6 antibody include those which are commercially available, or have been described in WO2021113697A1 (such as, h2A2, of which the heavy chain is shown as SEQ ID NO: 264, and the light chain is shown as SEQ ID NO: 265), which is herein incorporated by reference in their entirety.

Exemplary antibodies include 3F8, abagovomab, abciximab (REOPRO), adalimumab (HUMIRA), adecatumumab, afelimomab, afutuzumab, alacizumab, ALD518, alemtuzumab (CAMPATH), altumomab, amatuximab, anatumomab, anrukinzumab, apolizumab, arcitumomab (CEA-SCAN), aselizumab, atlizumab (tocilizumab, Actemra, RoActemra), atorolimumab, bapineuzumab, basiliximab (Simulect), bavituximab, bectumomab (LYMPHOSCAN), belimumab (BENLYSTA), benralizumab, bertilimumab, besilesomab (SCINITIMUN), bevacizumab (AVASTIN), biciromab (FIBRISCINT), bivatuzumab, blinatumomab, brentuximab, briakinumab, canakinumab (ILARIS), cantuzumab, capromab, catumaxomab (REMOVAB), CC49, cedelizumab, certolizumab, cetuximab (ERBITUX), citatuzumab, cixutumumab, clenoliximab, clivatuzumab, conatumumab, CR6261, dacetuzumab, daclizumab (ZENAPAX), daratumumab, denosumab (PROLIA), detumomab, dorlimomab, dorlixizumab, ecromeximab, eculizumab (SOLIRIS), edobacomab, edrecolomab (PANOREX), efalizumab (RAPTIVA), efungumab (MYCOGRAB), elotuzumab, elsilimomab, enlimomab, epitumomab, epratuzumab, erlizumab, ertumaxomab (REXOMUN), etaracizumab (ABEGRIN), exbivirumab, fanolesomab (NEUTROSPEC), faralimomab, farletuzumab, felvizumab, fezakinumab, figitumumab, fontolizumab (HuZAF), foravirumab, fresolimumab, galiximab, gantenerumab, gavilimomab, gemtuzumab, girentuximab, glembatumumab, golimumab (SIMPONI), gomiliximab, ibalizumab, ibritumomab, igovomab (INDIMACIS-125), imeiromab (MYOSCINT), infliximab (REMICADE), intetumumab, inolimomab, inotuzumab, ipilimumab, iratumumab, keliximab, labetuzumab (CEA-CIDE), lebrikizumab, lemalesomab, lerdelimumab, lexatumumab, libivirumab, lintuzumab, lucatumumab, lumiliximab, mapatumumab, maslimomab, matuzumab, mepolizumab (BOSATRIA), metelimumab, milatuzumab, minretumomab, mitumomab, morolimumab, motavizumab (NUMAX), muromonab-CD3 (ORTHOCLONE OKT3), nacolomab, naptumomab, natalizumab (TYSABRI), nebacumab, necitumumab, nerelimomab, nimotuzumab (THERACIM), nofetumomab, ocrelizumab, odulimomab, ofatumumab (ARZERRA), olaratumab, omalizumab (XOLAIR), ontecizumab, oportuzumab, oregovomab (OVAREX), otelixizumab, pagibaximab, palivizumab (SYNAGIS), panitumumab (VECTIBIX), panobacumab, pascolizumab, pemtumomab (THERAGYN), pertuzumab (OMNITARG), pexelizumab, pintumomab, priliximab, pritumumab, PRO 140, rafivirumab, ramucirumab, ranibizumab (LUCENTIS), raxibacumab, regavirumab, reslizumab, rilotumumab, rituximab (RITUXAN), robatumumab, rontalizumab, rovelizumab (LEUKARREST), ruplizumab (ANTOVA), acituzumab, satumomab pendetide, sevirumab, sibrotuzumab, sifalimumab, siltuximab, siplizumab, solanezumab, sonepcizumab, sontuzumab, stamulumab, sulesomab (LEUKOSCAN), tacatuzumab (AFP-CIDE), tetraxetan, tadocizumab, talizumab, tanezumab, taplitumomab paptox, tefibazumab (AUREXIS), telimomab, tenatumomab, teneliximab, teplizumab, TGN1412, ticilimumab (tremelimumab), tigatuzumab, TNX-650, tocilizumab (atlizumab, ACTEMRA), toralizumab, tositumomab (BEXXAR), trastuzumab (HERCEPTIN), tremelimumab, tucotuzumab, tuvirumab, urtoxazumab, ustekinumab (STELERA), vapaliximab, vedolizumab, veltuzumab, vepalimomab, visilizumab (NUVION), volociximab (HUMASPECT), votumumab, zalutumumab (HuMEX-EGFr), zanolimumab (HuMAX-CD4), ziralimumab and zolimomab.

In some embodiments, the antibodies are directed to cell surface markers for 5T4, CA-125, CEA, CD3, CD19, CD20, CD22, CD30, CD33, CD40, CD44, CD51, CTLA-4, EpCAM, HER2, EGFR (HER1), FAP, folate receptor, HGF, integrin αvβ3, integrin α5β1, IGF-1 receptor, GD3, GPNMB, mucin, MUC1, phosphatidylserine, prostatic carcinoma cells, PDGFR α, TAG-72, tenascin C, TRAIL-R2, VEGF-A and VEGFR2. In this embodiment the antibodies are abagovomab, adecatumumab, alacizumab, altumomab, anatumomab, arcitumomab, bavituximab, bevacizumab (AVASTIN), bivatuzumab, blinatumomab, brentuximab, cantuzumab, catumaxomab, capromab, cetuximab, citatuzumab, clivatuzumab, conatumumab, dacetuzumab, edrecolomab, epratuzumab, ertumaxomab, etaracizumab, farletuzumab, figitumumab, gemtuzumab, glembatumumab, ibritumomab, igovomab, intetumumab, inotuzumab, labetuzumab, lexatumumab, lintuzumab, lucatumumab, matuzumab, mitumomab, naptumomab estafenatox, necitumumab, oportuzumab, oregovomab, panitumumab, pemtumomab, pertuzumab, pritumumab, rituximab (RITUXAN), rilotumumab, robatumumab, satumomab, sibrotuzumab, taplitumomab, tenatumomab, tenatumomab, ticilimumab (tremelimumab), tigatuzumab, trastuzumab (HERCEPTIN), tositumomab, tremelimumab, tucotuzumab celmoleukin, volociximab and zalutumumab.

In certain embodiments, the antibodies directed to cell surface markers for HER2 are pertuzumab or trastuzumab and for EGFR (HER1) the antibody is cetuximab or panitumumab; and for CD20 the antibody is rituximab and for VEGF-A is bevacizumab and for CD-22 the antibody is epratuzumab or veltuzumab and for CEA the antibody is labetuzumab.

Exemplary peptides or peptide mimics include integrin targeting peptides (RGD peptides), LHRH receptor targeting peptides, ErbB2 (HER2) receptor targeting peptides, prostate specific membrane bound antigen (PSMA) targeting peptides, lipoprotein receptor LRP1 targeting, ApoE protein derived peptides, ApoA protein peptides, somatostatin receptor targeting peptides, chlorotoxin derived peptides, and bombesin.

In specific embodiments the peptides or peptide mimics are LHRH receptor targeting peptides and ErbB2 (HER2) receptor targeting peptides.

Exemplary proteins comprise insulin, transferrin, fibrinogen-gamma fragment, thrombospondin, claudin, apolipoprotein E, Affibody molecules such as, for example, ABY-025, Ankyrin repeat proteins, ankyrin-like repeats proteins and synthetic peptides.

In some embodiments, the targeting moiety-linker-polymer-oligonucleotide conjugates comprise broad spectrum cytotoxins in combination with cell surface markers for HER2 such as pertuzumab or trastuzumab; for EGFR such as cetuximab and panitumumab; for CEA such as labetuzumab; for CD20 such as rituximab; for VEGF-A such as bevacizumab; or for CD-22 such as epratuzumab or veltuzumab.

In other embodiments, the targeting moiety-linker-oligonucleotide-polymer conjugates comprise combinations of two or more targeting moieties, such as, for example, combination of bispecific antibodies directed to the EGF receptor (EGFR) on tumor cells and to CD3 and CD28 on T cells; combination of antibodies or antibody derived from Fab, Fab2, scFv or camel antibody heavy-chain fragments and peptides or peptide mimetics; combination of antibodies or antibody derived from Fab, Fab2, scFv or camel antibody heavy-chain fragments and proteins; combination of two bispecific antibodies such as CD3×CD19 plus CD28×CD22 bispecific antibodies.

In other embodiments, the targeting moiety-linker-oligonucleotide-polymer conjugates comprise targeting moieties which are antibodies against antigens, such as, for example, Sacituzumab, Trastuzumab, Cetuximab, Rituximab, Bevacizumab, Epratuzumab, Veltuzumab, Labetuzumab, B7-H4, B7-H3, CA125, CD33, CXCR2, EGFR, FGFR1, FGFR2, FGFR3, FGFR4, HER2, NaPi2b, c-Met, NOTCH1, NOTCH2, NOTCH3, NOTCH4, PD-L1, c-Kit, MUC1 and 5T4.

In certain embodiments, the targeting moiety-linker-oligonucleotide-polymer conjugates comprise targeting moieties which are antibodies against 5T4, such as, for example a humanized anti-5T4 scFvFc antibody.

Examples of suitable 5T4 targeting ligands or immunoglobulins include those which are commercially available, or have been described in the patent or non-patent literature, e.g., U.S. Pat. Nos. 8,044,178, 8,309,094, 7,514,546, EP1036091 (commercially available as TroVax™, Oxford Biomedica), EP2368914A1, WO 2013041687 A1 (Amgen), US 2010/0173382, and P. Sapra, et al., Mol. Cancer Ther. 2013, 12:38-47. An anti-5T4 antibody is disclosed in U.S. Provisional Application No. 61/877,439, filed Sep. 13, 2013 and U.S. Provisional Application No. 61/835,858, filed Jun. 17, 2013. The contents of each of the patent documents and scientific publications are herein incorporated by reference in their entireties.

As used herein, the term “5T4 antigen-binding portion” refers to a polypeptide sequence capable of selectively binding to a 5T4 antigen. In exemplary conjugates, the 5T4 antigen-binding portion generally comprises a single chain scFv-Fc form engineered from an anti-5T4 antibody. A single-chain variable fragment (scFv-Fc) is a fusion protein of the variable regions of the heavy (VH) and light chains (VL) of an immunoglobulin, connected with a linker peptide, and further connected to an Fc region comprising a hinge region and CH2 and CH3 regions of an antibody (any such combinations of antibody portions with each other or with other peptide sequences is sometimes referred to herein as an “immunofusion” molecule). Within such a scFvFc molecule, the scFv section may be C-terminally linked to the N-terminus of the Fc section by a linker peptide.

In some embodiments, the Fv portion of the 5T4 antigen-binding portion may be engineered by well-known molecular biology techniques to comprise one or more amino acid substitutions in the VH region. The Fe portion of the 5T4 antigen binding portion preferably comprises a polypeptide sequence engineered from the human hinge, CH2 and CH3 regions of an anti-5T4 antibody.

### Conjugate

In one aspect, there is provided a polymeric scaffold of Formula (III) or Formula (IV):

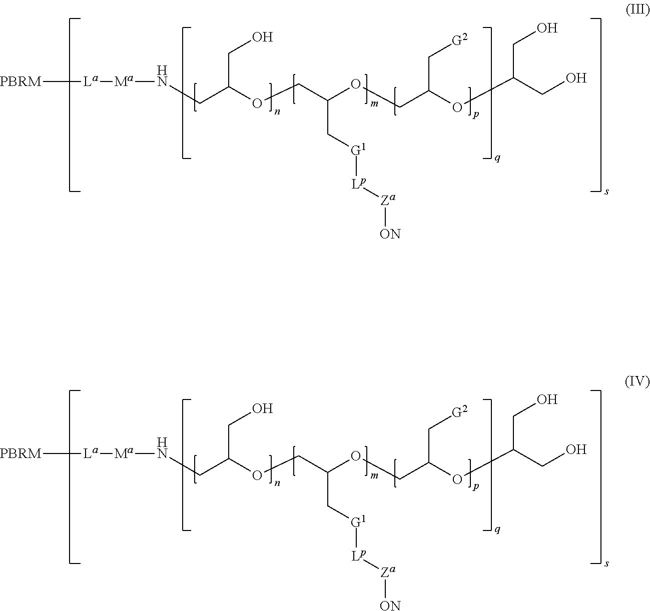

wherein
the polymeric scaffold comprises linear polyglycerol;
PBRM is a targeting moiety;
each L^{a} is independently a divalent moiety connecting the targeting moiety to M^{a};
each M^{a} is independently a stretcher connecting L^{a} to B^{a} or —NH— moiety;
B^{a} is a branching moiety comprising a functional group W_{M} connecting to —NH-moiety;
each G¹ is independently a functional group connecting L^{p} to the linear polyglycerol;
each L^{P} is independently a drug release mechanism between G¹ and Z^{a};
each Z^{a} is independently an endosomal-lysosomal escaping moiety or a conjugation moiety;
each ON is independently an oligonucleotide;
each G² is independently a functional group capable of converting into a charged state;
n is an integer from 0 to 1000;
m is an integer from 0 to 1000;
p is an integer from 0 to 1000;
q is an integer from 0 to 1000; and
s is an integer from 1 to 8.

In some embodiments, n is an integer from 0 to 1000, from 0 to 500, from 0 to 400, from 0 to 300, from 0 to 200, from 0 to 100, from 0 to 90, from 0 to 80, from 0 to 70, from 0 to 60, from 0 to 50, from 0 to 40, from 0 to 30, from 0 to 20, from 0 to 10, from 0 to 9, from 0 to 8, from 0 to 7, from 0 to 6, from 0 to 5, from 0 to 4, from 0 to 3, or from 0 to 2.

In some embodiments, m is an integer from 0 to 1000, from 0 to 500, from 0 to 400, from 0 to 300, from 0 to 200, from 0 to 100, from 0 to 90, from 0 to 80, from 0 to 70, from 0 to 60, from 0 to 50, from 0 to 40, from 0 to 30, from 0 to 20, from 1 to 10, from 1 to 9, from 1 to 8, from 1 to 7, from 1 to 6, from 1 to 5, from 1 to 4, from 1 to 3, or from 1 to 2.

In some embodiments, p is an integer from 0 to 1000, from 0 to 500, from 0 to 400, from 0 to 300, from 0 to 200, from 0 to 100, from 0 to 90, from 0 to 80, from 0 to 70, from 0 to 60, from 0 to 50, from 0 to 40, from 0 to 30, from 0 to 20, from 1 to 10, from 1 to 9, from 1 to 8, from 1 to 7, from 1 to 6, from 1 to 5, from 1 to 4, from 1 to 3, or from 1 to 2.

In some embodiments, q is an integer from 0 to 1000, from 0 to 500, from 0 to 400, from 0 to 300, from 0 to 200, from 0 to 100, from 0 to 90, from 0 to 80, from 0 to 70, from 0 to 60, from 0 to 50, from 0 to 40, from 0 to 30, from 0 to 20, from 1 to 10, from 1 to 9, from 1 to 8, from 1 to 7, from 1 to 6, from 1 to 5, from 1 to 4, from 1 to 3, or from 1 to 2.

In some embodiments, s is an integer from 1 to 8, from 1 to 7, from 1 to 6, from 1 to 5, from 1 to 4, from 1 to 3 or from 1 to 2.

In some embodiments, Wₚ is capable of reacting with a functional group on the targeting moiety with a click reaction.

In certain embodiments, Wₚ is selected from the group consisting of:

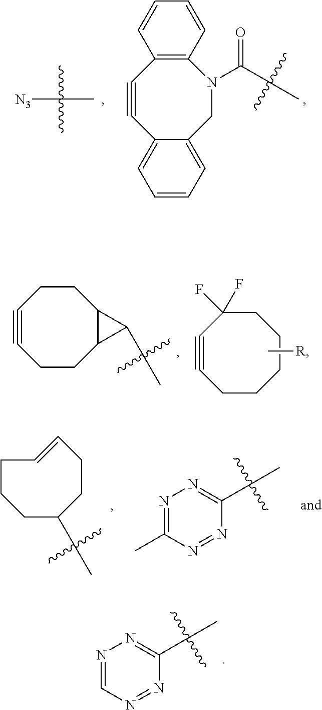

In some embodiments, Wₚ is capable of reacting with amino acids on the targeting moiety.

In certain embodiments, Wₚ is capable of reacting with amino acids on the targeting moiety, and the amino acids are natural amino acids, non-natural amino acids or combination thereof. In certain embodiments, the natural amino acid may comprise cysteine, lysine, tyrosine, aspartic acid and glutamic acid.

In some embodiments, Wₚ is capable of reacting with one or more cysteines on the targeting moiety.

In certain embodiments, Wₚ is capable of reacting with one or more cysteines on the targeting moiety and each Wₚ is selected from the group consisting of:

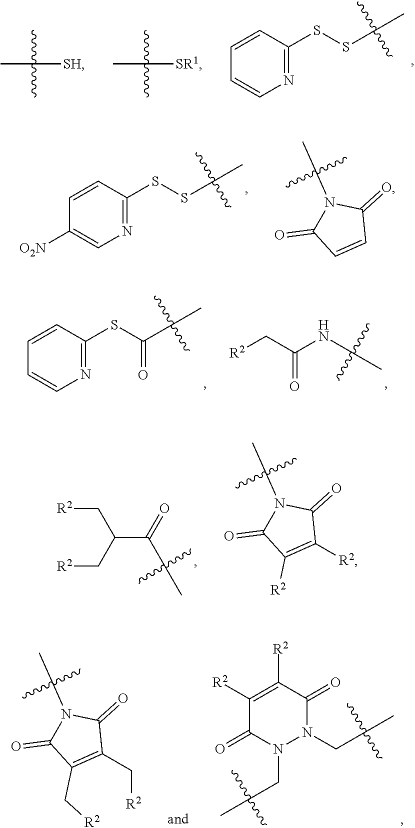

wherein R¹ is a sulfur protecting group, and each R² is independently a leaving group.

In certain embodiments, each R² is independently selected from halo or R^{2a}C(O)O—, in which R^{2a} is hydrogen, aliphatic, heteroaliphatic, cycloalkyl, heterocycloalkyl, aryl or heteroaryl.

In some embodiments, Wₚ is capable of reacting with one or more lysines on the targeting moiety.

In certain embodiments, Wₚ is capable of reacting with one or more lysines on the targeting moiety, and each Wₚ is independently selected from the group consisting of:

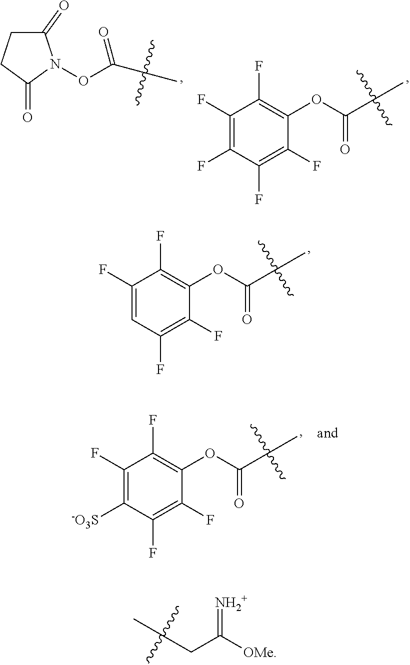

In some embodiments, Wₚ is capable of reacting with one or more non-natural amino acids on the targeting moiety.

In certain embodiments, Wₚ is capable of reacting with one or more non-natural amino acids on the targeting moiety and each Wₚ is independently selected from:

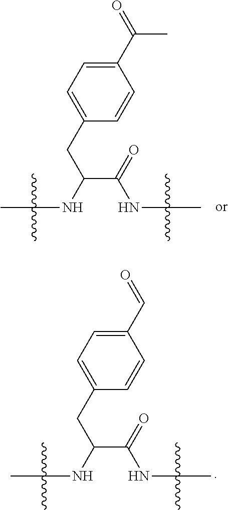 [SMILES: *NC(=O)C(Cc1ccc(C(C)=O)cc1)N* ; *NC(=O)C(Cc1ccc(C=O)cc1)N*]

In some embodiments, L is

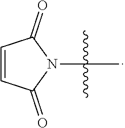 [SMILES: *N1C(=O)C=CC1=O]

In some embodiments, M^{a} is selected from the group consisting of:

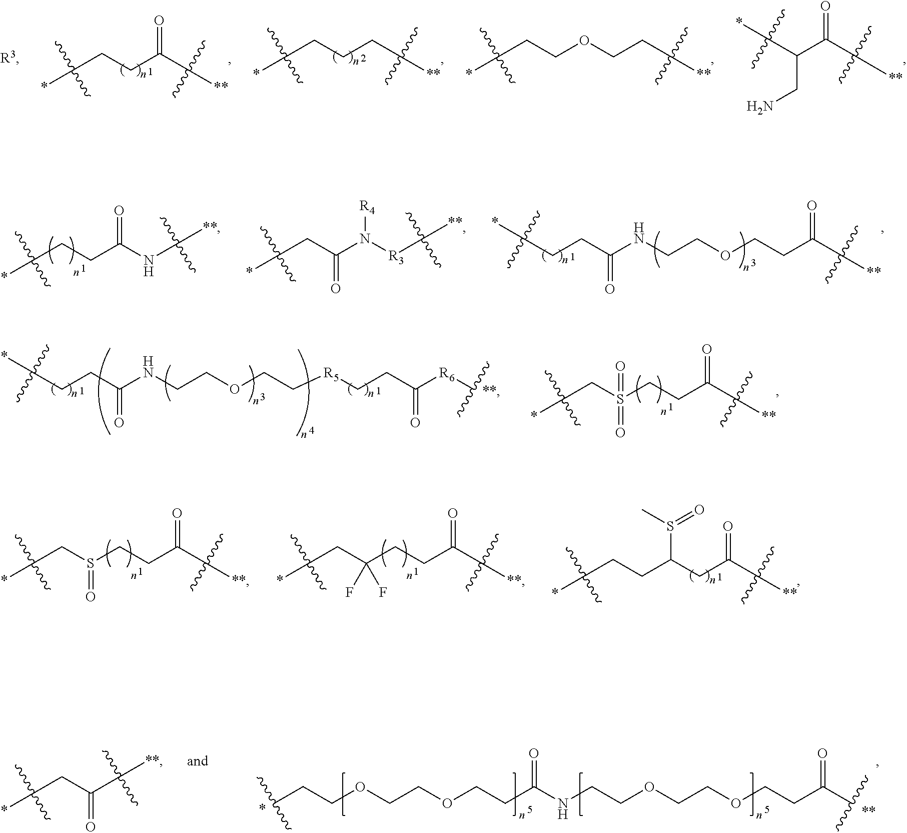

wherein
* is the site covalently attached to L, ** is the site covalently attached to B^{a} or —NH— moiety;
R³ is C₁₋₁₀ alkyl, C₁₋₁₀ heteroalkyl, C₃₋₈cycloalkyl, —O—(C₁₋₈ alkyl)-, aryl, —C₁₋₁₀ alkyl-aryl-, -aryl-C₁₋₁₀ alkyl-, —C₁₋₁₀ alkyl-(C₃₋₈cycloalkyl)-, —(C₃₋₈cycloalkyl-C₁₋₁₀ alkyl)-, 4- to 14-membered heterocycloalkyl, —C₁₋₁₀ alkyl-(4- to 14-membered heterocycloalkyl)-, -(4- to 14-membered heterocycloalkyl)-C₁₋₁₀ alkyl-, —C₁₋₁₀ alkyl-C(═O)—, —C₁₋₁₀ heteroalkyl-C(═O)—, —C₃₋₈cycloalkyl-C(═O)—, —O—(C₁₋₈alkyl)-C(═O)—, -aryl-C(═O)—, —C₁₋₁₀ alkyl-aryl-C(═O)—, -aryl-C₁₋₁₀ alkyl-C(═O)—, —C₁₋₁₀ alkyl-(C₃₋₈cycloalkyl)-C(═O)—, —(C₃₋₈ cycloalkyl)-C₁₋₁₀ alkyl-C(═O)—, -4- to 14-membered heterocycloalkyl-C═(O)—, —C₁₋₁₀ alkyl-(4- to 14-membered heterocycloalkyl)-C(═O)—, -(4- to 14-membered heterocycloalkyl)-C₁₋₁₀ alkyl-C(═O)—, —C₁₋₁₀ alkyl-NH—, —C₁₋₁₀ heteroalkyl-NH—, —C₃₋₈cycloalkyl-NH—, —O—(C₁₋₈alkyl)-NH—, -aryl-NH—, —C₁₋₁₀ alkyl-aryl-NH—, -aryl-C₁₋₁₀ alkyl-NH—, —C₁₋₁₀ alkyl-(C₃₋₈ cycloalkyl)-NH—, —(C₃₋₈ cycloalkyl)-C₁₋₁₀ alkyl-NH—, -4- to 14-membered heterocycloalkyl-NH—, —C₁₋₁₀ alkyl-(4- to 14-membered heterocycloalkyl)-NH—, -(4- to 14-membered heterocycloalkyl)-C₁₋₁₀ alkyl-NH—, —C₁₋₁₀ alkyl-S—, —C₁₋₁₀ heteroalkyl-S—, —C₃₋₈ cycloalkyl-S—, —O—C₁₋₈ alkyl-S—, -aryl-S—, —C₁₋₁₀ alkyl-aryl-S—, -aryl-C₁₋₁₀ alkyl-S—, —C₁₋₁₀ alkyl-(C₃₋₈cycloalkyl)-S—, —(C₃₋₈cycloalkyl)-C₁₋₁₀ alkyl-S—, -4- to 14-membered heterocycloalkyl-S—, —C₁₋₁₀ alkyl-(4- to 14-membered heterocycloalkyl)-S—, or -(4- to 14-membered heterocycloalkyl)-C₁₋₁₀ alkyl-S—;
each R⁴ independently is hydrogen, C₁₋₆ alkyl, C₆₋₁₀ aryl, C₃₋₈ cycloalkyl, —COOH or —COO—C₁₋₆ alkyl;
R⁵ is —C(O)—NR^{5a} or —NR^{5a}—C(O)—;
R^{5a} is hydrogen, C₁₋₆ alkyl, C₆₋₁₀ aryl, C₃₋₈ cycloalkyl, —COOH or —COO—C₁₋₆ alkyl;
R⁶ is a bond or —NR^{6a}—(CR^{6b}R^{6c})—C(O)—;
R^{6a} is hydrogen, C₁₋₆ alkyl, C₆₋₁₀ aryl, C₃₋₈ cycloalkyl, —COOH or —COO—C₁₋₆ alkyl;
each R^{6b} and R^{6c} independently is hydrogen, C₁₋₆ alkyl, C₆₋₁₀ aryl, hydroxylated C₆₋₁₀ aryl, polyhydroxylated C₆₋₁₀ aryl, 5- to 12-membered heterocycloalkyl, C₃₋₈ cycloalkyl, hydroxylated C₃₋₈ cycloalkyl, polyhydroxylated C₃₋₈ cycloalkyl or a side chain of a natural or unnatural amino acid;
each n¹ independently is an integer from 0 to 6;
n² is an integer from 0 to 8;
each n³ independently is an integer from 1 to 6;
n⁴ is an integer from 1 to 4; and
n⁵ is an integer from 1 to 4.

In certain embodiments, M^{a} is selected from the group consisting of:

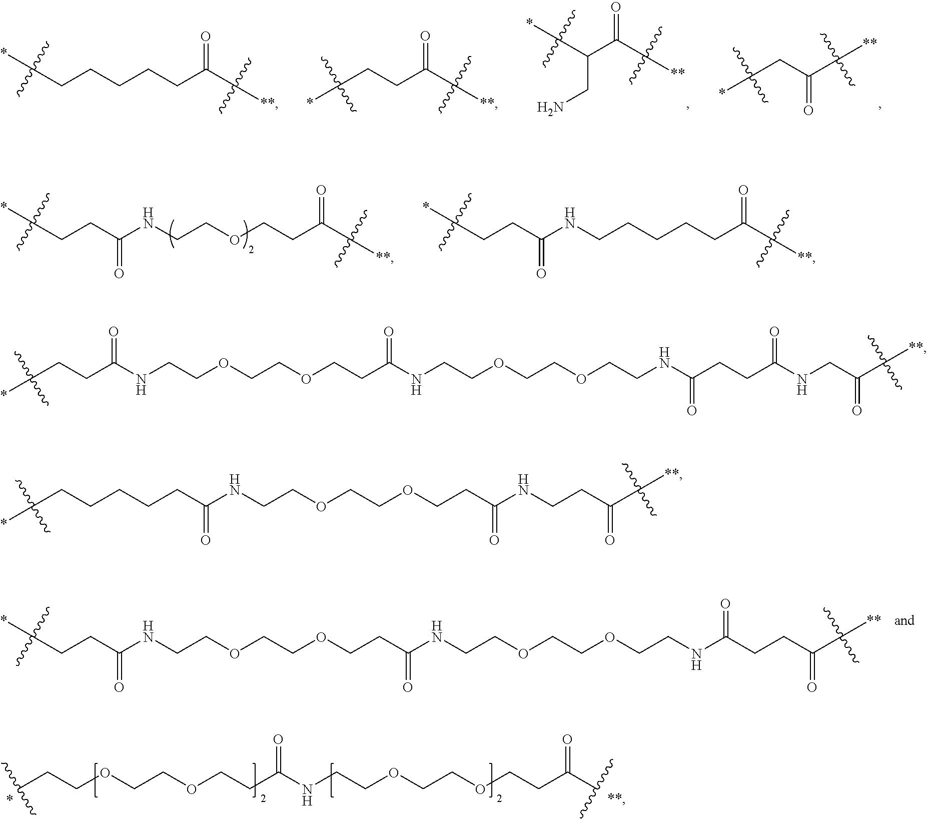

wherein * is the site covalently attached to L, ** is the site covalently attached to B^{a} or —NH— moiety.

In some embodiments, B^{a} is

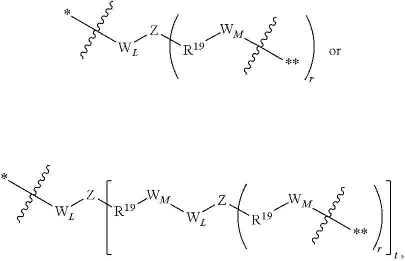

wherein W_{L} is a functional group connected to stretcher M^{a};
each Z is independently a branching point;
each R¹⁹ is independently a linker connecting Z to W_{M};
each W_{M} is independently a functional group connected to —NH—;
r is an integer from 1-3;
t is an integer from 1-3;
wherein * is the site covalently attached to M^{a}, ** is the site covalently attached to —NH— moiety.

In some embodiments, the —NH— moiety is

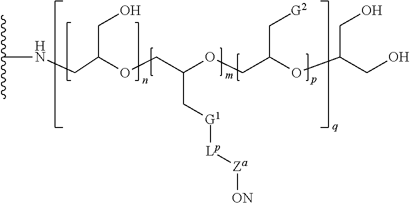

or —NH—(CH₂)ₙ₆—COOH, wherein G¹, L^{p}, Z^{a}, G², ON, n, m, p and q are as defined supra, and n⁶ is an integer from 1 to 6.

In certain embodiments, W_{L} is selected from a group consisting of:

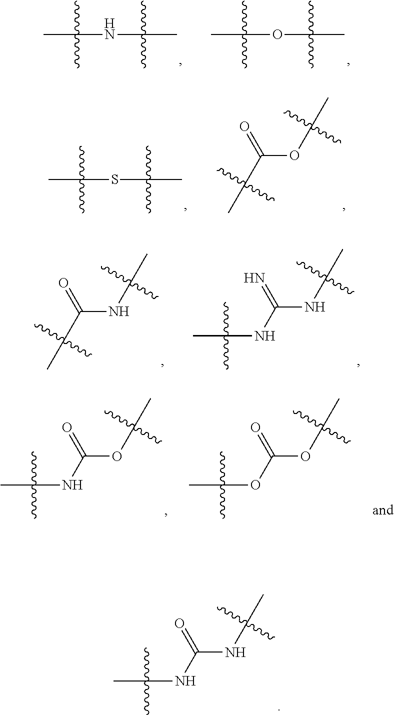

In certain embodiments, Z is —CH₍₃₋ᵣ₎-, -SiH₍₃₋ᵣ₎— or -NH₍₂₋ᵣ₎—.

In certain embodiments, R¹⁹ is selected from a group consisting of an aliphatic, heteroaliphatic, cycloalkyl, and heterocycloalkyl. In certain embodiments, R¹⁹ is alkyl or heteroalkyl.

In some embodiments, B^{a} is

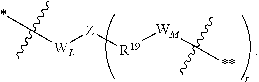

In certain embodiments, B^{a} is selected from the group consisting of:

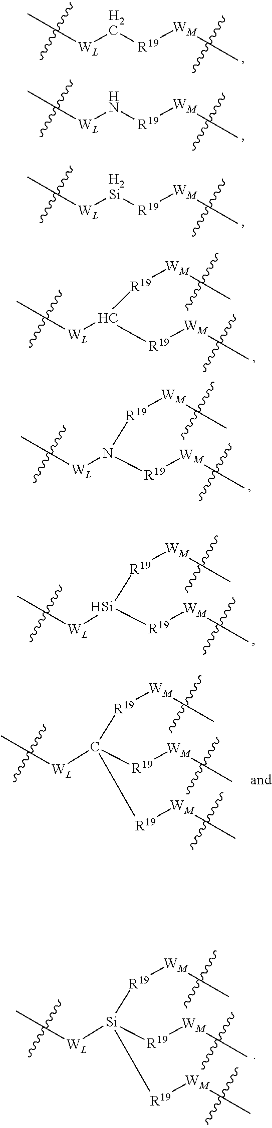

In some embodiments, B^{a} is

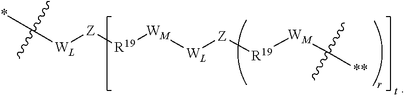

In certain embodiments, B^{a} is selected from the group consisting of:

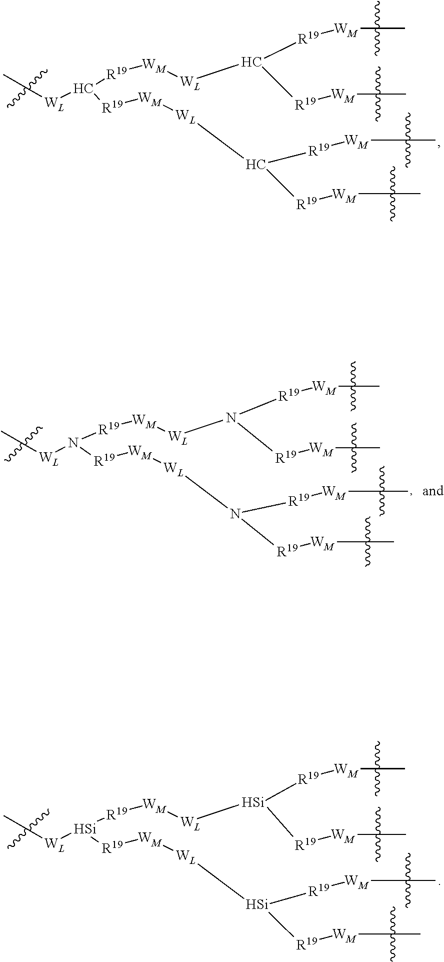

In some embodiments, B^{a} is

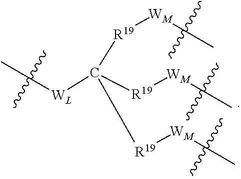

In some embodiments, two of the W_{M} are connected to —NH—(CH₂)ₙ₆—COOH, and one of the W_{M} is connected to

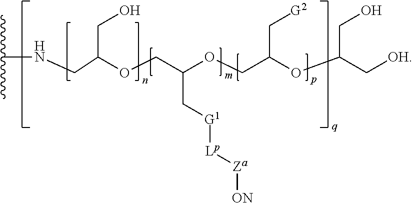

In some embodiments, three W_{M} are connected to

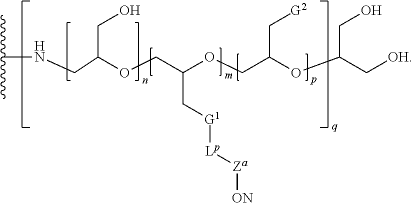

In certain embodiments, W_{L} is

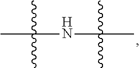 [SMILES: *N*]

and —R¹⁹—W_{M}— is

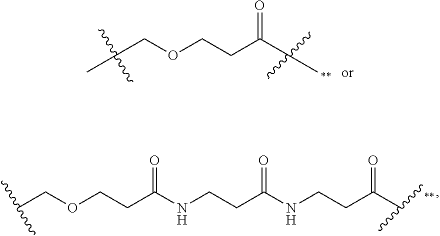 [SMILES: *COCCC(*)=O ; *COCCC(=O)NCCC(=O)NCCC(*)=O]

wherein ** is the site covalently attached to W_{L} or —NH— moiety.

In certain embodiments, B^{a} is

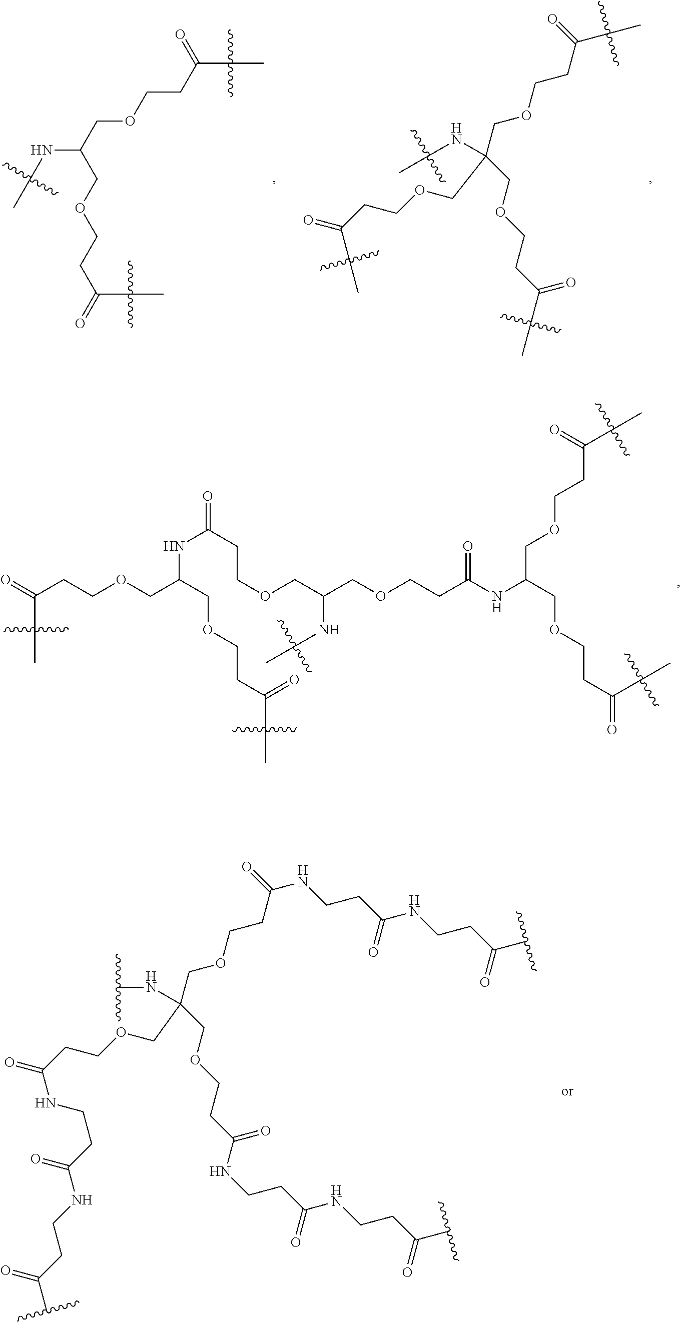 [SMILES: *NC(COCCC(*)=O)COCCC(*)=O ; *NC(COCCC(*)=O)(COCCC(*)=O)COCCC(*)=O ; *NC(COCCC(=O)NC(COCCC(*)=O)COCCC(*)=O)COCCC(=O)NC(COCCC(*)=O)COCCC(*)=O ; *NC(COCCC(=O)NCCC(=O)NCCC(*)=O)(COCCC(=O)NCCC(=O)NCCC(*)=O)COCCC(=O)NCCC(=O)NCCC(*)=O]

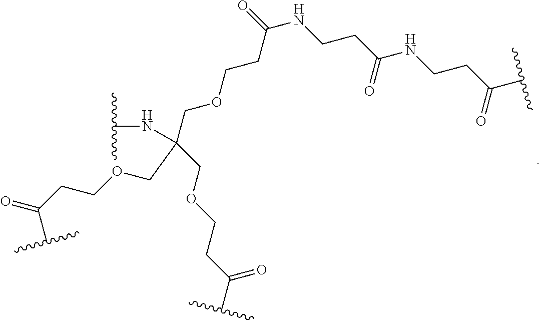 [SMILES: *NC(COCCC(*)=O)(COCCC(*)=O)COCCC(=O)NCCC(=O)NCCC(*)=O]

In some embodiments, G¹ is selected from the group consisting of:

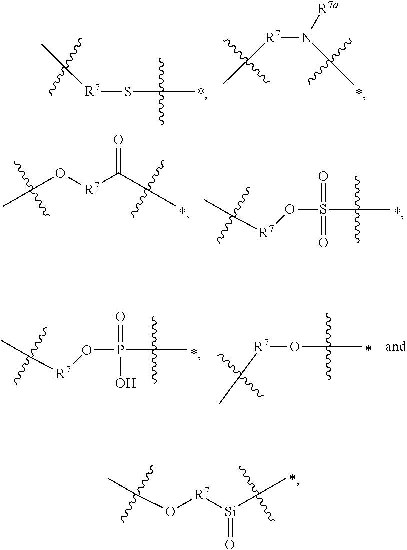

wherein * is the site covalently attached to L^{P}, each R⁷ is independently selected from a direct bond, alkyl, cycloalkyl, heterocycloalkyl, aryl, or heteroaryl, and R^{7a} is selected from hydrogen, an aliphatic, heteroaliphatic, cycloalkyl, or heterocycloalkyl moiety.

In some embodiments, each L independently comprises a labile structure.

In certain embodiments, the labile structure is selected from redox labile structures, hydrolytically labile structures or enzymatic labile structures.

In certain embodiments, the labile structure is a redox labile structure having a structure of

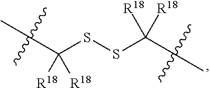

wherein each of R¹⁸ is independently selected from hydrogen, aliphatic, heteroaliphatic, cycloalkyl or heterocycloalkyl.

In certain embodiments, the hydrolytically labile structure is selected from the group consisting of:

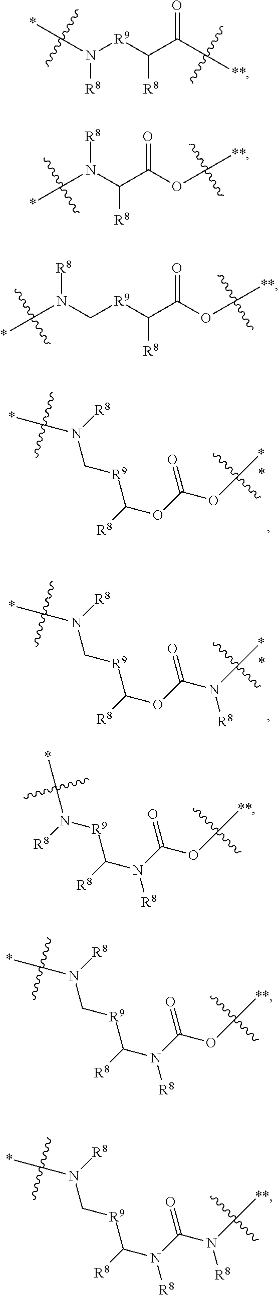

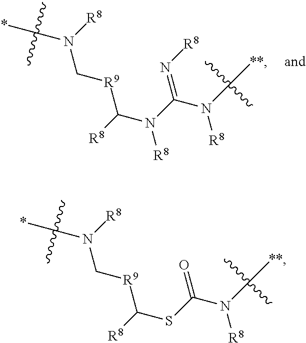

wherein * is the site covalently attached to G¹, ** is the site covalently attached to Z^{a}, R⁸ is selected from hydrogen, alky or aryl, R⁹ is selected from aliphatic, heteroaliphatic, cycloalkyl, heterocycloalkyl, aryl, or heteroaryl.

In certain embodiments, G¹ is

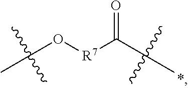

wherein * is the site covalently attached to L^{P}, and R⁷ is alkyl (such as C₁₋₆ alkyl).

In some embodiments, the enzymatic labile structure is liable to enzymes selected from Cathepsin B, phosphatase, sulfatase, or glucuronidase.

In certain embodiments, the enzymatic labile structure is liable to cathepsin B and is selected from —Z— or

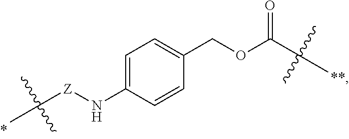

wherein * is the site covalently attached to G¹, ** is the site covalently attached to Z^{a}, and Z is a substrate for cathepsin B comprising 2 to 4 amino acids.

In certain embodiments, G¹ is

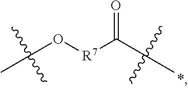

wherein * is the site covalently attached to L^{P}, and R⁷ is alkyl (such as C₁₋₆ alkyl).

In certain embodiments, the enzymatic labile structure is liable to glucuronidase ad is

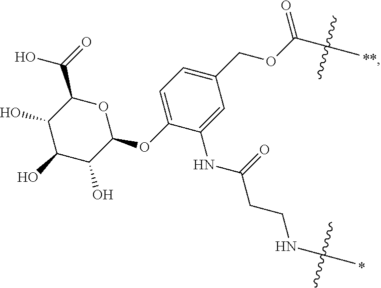 [SMILES: *NCCC(=O)Nc1cc(COC(*)=O)ccc1O[C@@H]1O[C@H](C(=O)O)[C@@H](O)[C@H](O)[C@H]1O]

wherein * is the site covalently attached to G¹, ** is the site covalently attached to Z^{a}.

In certain embodiments, G¹ is

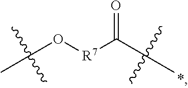

wherein * is the site covalently attached to L^{P}, and R⁷ is alkyl (such as C₁₋₆ alkyl).

In certain embodiments, the enzymatic labile structure is liable to phosphatase and is selected from

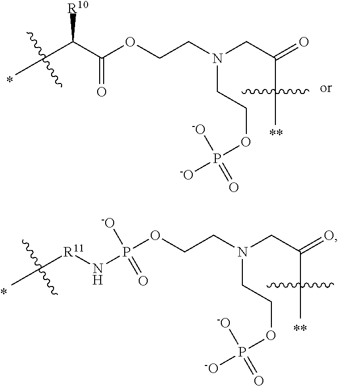

wherein * is the site covalently attached to G¹, ** is the site covalently attached to Z^{a}, and each of R¹⁰ and R¹¹ is independently hydrogen, aliphatic, heteroaliphatic, cycloalkyl, or heterocycloalkyl moiety.

In certain embodiments, G¹ is

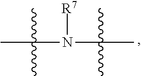

wherein R⁷ is selected from hydrogen, an aliphatic, heteroaliphatic, cycloalkyl, heterocycloalkyl, aryl or heteroaryl.

In certain embodiments, the enzymatic labile structures are liable to sulfatase and is

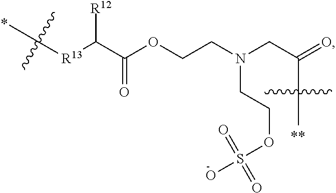

wherein * is the site covalently attached to G¹, ** is the site covalently attached to Z^{a}, and each of R¹² and R¹³ is independently hydrogen, —NH—, aliphatic, heteroaliphatic, cycloalkyl, or heterocycloalkyl moiety.

In some embodiments, G¹ is

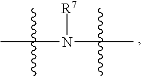

wherein R⁷ is selected from hydrogen, aliphatic, heteroaliphatic, cycloalkyl, or heterocycloalkyl moiety.

In some embodiments, Z^{a} is a cell penetration peptide.

In certain embodiments, Z^{a} is selected from the group consisting of Tat, Penetratin, Pep-1, MPG, Polyarginine (R9, R8), R6/W3, SAISAP(E), CyLop-1, gH 625, GALA, TP10, CADY, L17E, MPPs, Ac-1, Ent Ac-1, Ac-2, Peptide 3, RR5-App, RR4-App, RR3-aPP, TATp-D, R4-R4, R5-R5, [WR]₄, Cyclic Tat, cFΦR4, Danamide D, Pro-(Xaa)₄-Tyr, Cyclic sC18, RRRRqF, BIM SAHB9, SAH-SOS1, 4-R, 4-W, and Sp-CC-PEG₂₀₀₀.

In some embodiments, Z^{a} is an endosomal escape domain.

In certain embodiments, Z^{a} is selected from the group consisting of GG, GFFG, GFWFG, GWGGWG, GWG, GFWG, GWWG, and GWWG.

In some embodiments, Z^{a} is a cationic polymer.

In certain embodiments, Z^{a} is selected from PEI, PLL, PLA, DEAE-DEX or PAMAM. In certain embodiments, Z^{a} is PEI.

In some embodiments, Z^{a} is a conjugation moiety.

In certain embodiments, Z^{a} is **—NH—(CH₂)ₙ₇—* or **—S—(CH₂)ₙ₇—*, wherein n⁷ is an integer from 1 to 6, * is the site covalently attached to ON, and ** is the site covalently attached to L^{p}.

In certain embodiments, Z^{a} is —NH—(CH₂)₆—.

In some embodiments, -G¹-L^{p}-Z^{a}— is

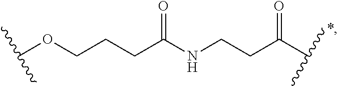 [SMILES: *OCCCC(=O)NCCC(*)=O]

wherein * is the site covalently attached to ON.

In some embodiments, n is an integer from 1 to 100; m is an integer from 1 to 100; and p is an integer from 1 to 50.

In some embodiments, ON is selected from the group consisting of antisense oligonucleotides, mRNA, siRNA, saRNA, miRNA, shRNA and CRISPER/Cas9 complex.

In certain embodiments, ON silences the TGFβ.

In certain embodiments, ON is a double strand siRNA.

In certain embodiments, sense strand sequence of the siRNA is shown as SEQ ID NO: 203, and antisense strand sequence of the siRNA is shown as SEQ ID NO: 204.

In certain embodiments, Z^{a} is connected to ON at 5′ or 3′ end.

In certain embodiments, G² is selected from the group consisting of:

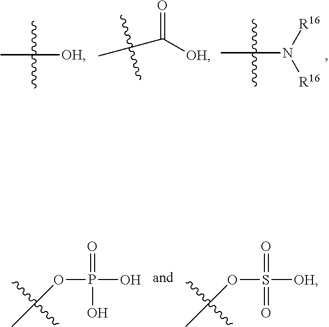

wherein each R¹⁶ is independently hydrogen, aliphatic, heteroaliphatic, cycloalkyl, heterocycloalkyl, aryl or heteroaryl. In certain embodiments, each R¹⁶ is independently hydrogen or alkyl. In certain embodiments, each R¹⁶ is independently hydrogen or C₁₋₆ alkyl.

In some embodiments, the targeting moiety is an antibody and/or fragment thereof.

In certain embodiments, the targeting moiety is an antibody IgG1, IgG2, IgG3, and IgG4.

In certain embodiments, the targeting moiety is selected from the group consisting of a F′b, a Fab′, a F(ab′)₂, a Fd, an Fv fragment, a disulfide stabilized Fv fragment (dsFv), a (dsFv)₂, a bispecific dsFv (‘sFv-dsFv’), a disulfide stabilized diabody (ds diabody), a single-chain antibody molecule (scFv), an scFv dimer, a bispecific antibody, a trispecific antibody, a bi-paratropic antibody, a multispecific antibody, a camelized single domain antibody, a nanobody, a domain antibody, a bivalent domain antibody, or VHH.

In certain embodiments, the targeting moiety is anti-αvβ6 antibody, or antigen-binding fragment thereof.

In certain embodiments, the targeting moiety is anti-αvβ6 antibody, wherein the sequence of heavy chain is shown as SEQ ID NO: 264, and the sequence of light chain is shown as SEQ ID NO: 265.

In some embodiments, the polymeric scaffold is

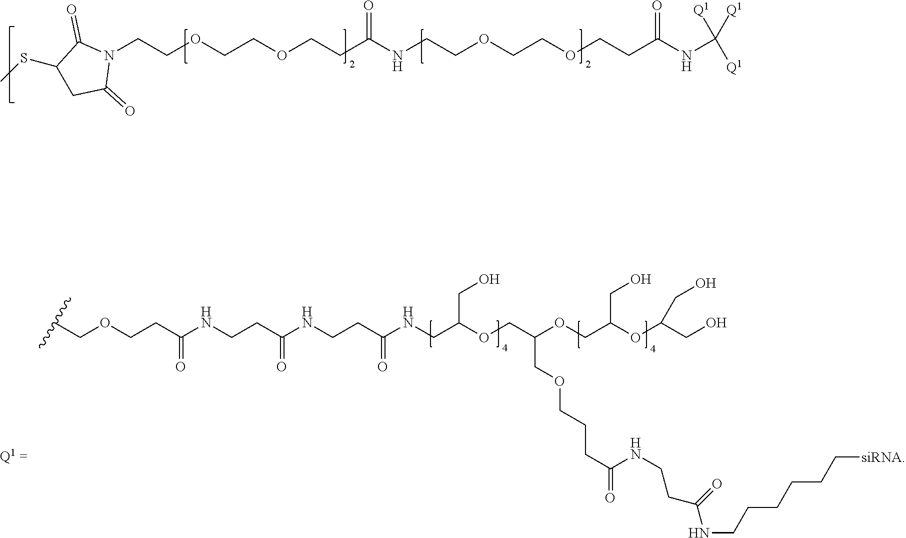

### Synthetic Methods

Any available techniques can be used to make the conjugates provided herein or compositions including them, and intermediates and components (e.g., carriers and modifiers) useful for making them. For example, semi-synthetic and fully synthetic methods may be used.

The synthetic processes of the disclosure can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt, ester or prodrug thereof.

In some embodiments, the linker-polymer compound provided herein can conjugate with both a targeting moiety and an oligonucleotide. The linker-polymer compound provided herein comprises a linking moiety suitable for connecting a targeting moiety and a linking moiety suitable for connecting an oligonucleotide.

In some embodiments, the conjugates provided herein are formed in several steps, including (1) modifying the polymer carrier so that the polymer carrier contains a functional group that can react with a functional group of the targeting moiety or its derivative and a functional group that can react with a functional group of the oligonucleotide or its derivative; (2) reacting the modified polymer with the oligonucleotide or its derivative so that the oligonucleotide is linked to the modified polymer; (3) reacting the modified polymer-oligonucleotide conjugate with the targeting moiety or its derivative to form the conjugate provided herein.

In another embodiment the conjugates are formed in several steps: (1) modifying the polymer carrier so that the polymer carrier contains a functional group that can react with a functional group of the targeting moiety or its derivative and a functional group that can react with a functional group of a first oligonucleotide or its derivative; (2) reacting the modified polymer with the first oligonucleotide or its derivative so that the first oligonucleotide is linked to the modified polymer; (3) modifying the resultant polymer-oligonucleotide conjugate so that it contains a different functional group that can react with a functional group of a second oligonucleotide or its derivative; (4) reacting the modified polymer-oligonucleotide conjugate with the second oligonucleotide or its derivative so that the second oligonucleotide is linked to the modified polymer-oligonucleotide conjugate; (5) reacting the modified polymer-oligonucleotide conjugate of step (4) with the targeting moiety or its derivative to form the conjugate provided herein.

The synthetic processes of the present disclosure can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt, ester or prodrug thereof.

Oligonucleotides used for the conjugates provided herein can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, incorporated by reference herein, are useful and recognized reference textbooks of organic synthesis known to those in the art.

Conjugates of the present disclosure can be conveniently prepared by a variety of methods familiar to those skilled in the art. The conjugates of the present disclosure with each of the formulae described herein may be prepared from commercially available starting materials or starting materials which can be prepared using literature procedures. The procedures show the preparation of representative conjugates of the present disclosure.

Conjugates designed, selected and/or optimized by methods described above, once produced, can be characterized using a variety of assays known to those skilled in the art to determine whether the conjugates have biological activity. For example, the conjugates can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the conjugate molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

### Pharmaceutical Composition

For the purposes of administration, in some embodiments, the conjugates provided herein are administered as a raw chemical or are formulated as pharmaceutical compositions.

Therefore, in one aspect, the present disclosure provides a pharmaceutical composition comprising one or more conjugates as disclosed herein and an acceptable carrier, such as a stabilizer, buffer, and the like. The conjugates can be administered and introduced into a subject by standard means, with or without stabilizers, buffers, and the like, to form a pharmaceutical composition. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal, oral or parenteral administration including intravenous, intraarterial, subcutaneous, intraperitoneal or intramuscular injection or infusion or intracranial, e.g., intrathecal or intraventricular, administration. The conjugates can be formulated and used as sterile solutions and/or suspensions for injectable administration; lyophilized powders for reconstitution prior to injection/infusion; topical compositions; as tablets, capsules, or elixirs for oral administration; or suppositories for rectal administration, and the other compositions known in the art.

A pharmacological composition or formulation refers to a composition or formulation in a form suitable for administration, e.g., systemic administration, into a cell or subject, including for example a human. Suitable forms, in part, depend upon the use or the route of entry, for example oral, inhaled, transdermal, or by injection/infusion. Such forms should not prevent the composition or formulation from reaching a target cell (i.e., a cell to which the oligonucleotide is desirable for delivery). For example, pharmacological compositions injected into the blood stream should be soluble. Other factors are known in the art, and include considerations such as toxicity and forms that prevent the composition or formulation from exerting its effect.

As used herein, the term “systemic administration” means in vivo systemic absorption or accumulation of the modified polymer in the blood stream followed by distribution throughout the entire body. Administration routes that lead to systemic absorption include, without limitation: intravenous, subcutaneous, intraperitoneal, inhalation, oral, intrapulmonary, and intramuscular. Each of these administration routes exposes the modified polymers to an accessible diseased tissue. The rate of entry of an active agent into the circulation has been shown to be a function of molecular weight or size. The use of a conjugate provided herein can localize the oligonucleotide delivery in certain cells, such as cancer cells via the specificity of targeting moieties.

As used herein, the term “pharmaceutically acceptable formulation” means a composition or formulation that allows for the effective distribution of the conjugates in the physical location most suitable for their desired activity. In some embodiments, effective delivery occurs before clearance by the reticuloendothelial system or the production of off-target binding which can result in reduced efficacy or toxicity. Non-limiting examples of agents suitable for formulation with the conjugates include: P-glycoprotein inhibitors (such as Pluronic P85), which can enhance entry of active agents into the CNS; biodegradable polymers, such as poly (DL-lactide-coglycolide) microspheres for sustained release delivery after intracerebral implantation; and loaded nanoparticles, such as those made of polybutylcyanoacrylate, which can deliver active agents across the blood brain barrier and can alter neuronal uptake mechanisms.

Also included herein are pharmaceutical compositions prepared for storage or administration, which include a pharmaceutically effective amount of the desired conjugates in a pharmaceutically acceptable carrier or diluent. Acceptable carriers, diluents, and/or excipients for therapeutic use are well known in the pharmaceutical art. For example, buffers, preservatives, bulking agents, dispersants, stabilizers, dyes, can be provided. In addition, antioxidants and suspending agents can be used Examples of suitable carriers, diluents and/or excipients include, but are not limited to: (1) Dulbecco's phosphate buffered saline, pH about 6.5, which would contain about 1 mg/ml to 25 mg/ml human serum albumin, (2) 0.9% saline (0.9% w/v NaCl), and (3) 5% (w/v) dextrose.

As used herein, the term “pharmaceutically effective amount” refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Pharmaceutically effective amounts for a given situation can be determined by routine experimentation that is within the skill and judgment of the clinician.

For any conjugate, the pharmaceutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The dosage may vary within this range depending upon the dosage form employed, sensitivity of the patient, and the route of administration.

For example, linker-polymer-oligonucleotide compound or targeting moiety-linker-polymer-oligonucleotide conjugates can be evaluated for their ability to inhibit tumor growth in several cell lines using Cell titer Glo. Dose response curves can be generated using SoftMax Pro software and IC₅₀ values can be determined from four-parameter curve fitting. Cell lines employed can include those which are the targets of the targeting moiety and a control cell line that is not the target of the targeting moiety contained in the test conjugates.

In some embodiments, the conjugates are formulated for parenteral administration by injection including using conventional catheterization techniques or infusion. Formulations for injection may be presented in unit dosage form, e.g., in ampules or in multi-dose containers, with an added preservative. The conjugates can be administered parenterally in a sterile medium. The conjugate, depending on the vehicle and concentration used, can either be suspended or dissolved in the vehicle. Advantageously, adjuvants such as local anesthetics, preservatives, and buffering agents can be dissolved in the vehicle. The term “parenteral” as used herein includes percutaneous, subcutaneous, intravascular (e.g., intravenous), intramuscular, or intrathecal injection or infusion techniques and the like. In addition, there is provided a pharmaceutical formulation comprising conjugates and a pharmaceutically acceptable carrier. One or more of the conjugates can be present in association with one or more non-toxic pharmaceutically acceptable carriers and/or diluents and/or adjuvants, and if desired other active ingredients.

The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, a bland fixed oil can be employed including synthetic mono- or diglycerides. In addition, fatty acids such as oleic acid find use in the preparation of injectables.

The conjugates and compositions described herein may be administered in appropriate form, preferably parenterally, more preferably intravenously. For parenteral administration, the conjugates or compositions can be aqueous or nonaqueous sterile solutions, suspensions or emulsions. Propylene glycol, vegetable oils and injectable organic esters, such as ethyl oleate, can be used as the solvent or vehicle. The compositions can also contain adjuvants, emulsifiers or dispersants.

Dosage levels of the order of from between about 0.001 mg and about 140 mg per kilogram of body weight per day are useful in the treatment of the above-indicated conditions (between about 0.05 mg and about 7 g per subject per day). In some embodiments, the dosage administered to a patient is between about 0.001 mg/kg to about 100 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight. The amount of conjugate that can be combined with the carrier materials to produce a single dosage form varies depending upon the host treated and the particular mode of administration. Dosage unit forms can generally contain from between about 0.001 mg and about 100 mg; between about 0.01 mg and about 75 mg; or between about 0.01 mg and about 50 mg; or between about 0.01 mg and about 25 mg; of a conjugate.

For intravenous administration, the dosage levels can comprise ranges described above, or from about 0.01 to about 200 mg of a conjugate per kg of the animal's body weight. In some embodiments, the composition can include from about 1 to about 100 mg of a conjugate per kg of the animal's body weight. In some embodiments, the amount administered will be in the range from about 0.1 to about 25 mg/kg of body weight of a compound.

In some embodiments, the conjugates can be administered are as follows. The conjugates can be given daily for about 5 days either as an i.v., bolus each day for about 5 days, or as a continuous infusion for about 5 days.

Alternatively, the conjugates can be administered once a week for six weeks or longer. As another alternative, the conjugates can be administered once every two or three weeks. Bolus doses are given in about 50 to about 400 ml of normal saline to which about 5 to about 10 ml of human serum albumin can be added. Continuous infusions are given in about 250 to about 500 ml of normal saline, to which about 25 to about 50 ml of human serum albumin can be added, per 24 hour period.

In some embodiments, about one to about four weeks after treatment, the patient can receive a second course of treatment. Specific clinical protocols with regard to route of administration, excipients, diluents, dosages, and times can be determined by the skilled artisan as the clinical situation warrants.

In other embodiments, the therapeutically effective amount may be provided on another regular schedule, i.e., daily, weekly, monthly, or yearly basis or on an irregular schedule with varying administration days, weeks, months, etc. Alternatively, the therapeutically effective amount to be administered may vary. In some embodiments, the therapeutically effective amount for the first dose is higher than the therapeutically effective amount for one or more of the subsequent doses. In some embodiments, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the recommendations of the relevant regulatory bodies and judgment of a health-care practitioner. The therapeutically effective amounts described herein refer to total amounts administered for a given time period; that is, if more than one different conjugate described herein is administered, the therapeutically effective amounts correspond to the total amount administered. It is understood that the specific dose level for a particular subject depends upon a variety of factors including the activity of the specific conjugate, the age, body weight, general health, sex, diet, time of administration, route of administration, and rate of excretion, combination with other active agents, and the severity of the particular disease undergoing therapy.

For administration to non-human animals, the conjugates can also be added to the animal feed or drinking water. It can be convenient to formulate the animal feed and drinking water so that the animal takes in a therapeutically appropriate quantity of the conjugates along with its diet. It can also be convenient to present the conjugates as a premix for addition to the feed or drinking water.

The conjugates can also be administered to a subject in combination with other therapeutic compounds to increase the overall therapeutic effect. The use of multiple compounds to treat an indication can increase the beneficial effects while reducing the presence of side effects. In some embodiments, the conjugates are used in combination with chemotherapeutic agents, such as those disclosed in U.S. Pat. No. 7,303,749. In other embodiments the chemotherapeutic agents, include, but are not limited to letrozole, oxaliplatin, docetaxel, 5-FU, lapatinib, capecitabine, leucovorin, erlotinib, pertuzumab, bevacizumab, and gemcitabine. The present disclosure also provides pharmaceutical kits comprising one or more containers filled with one or more of the conjugates and/or compositions of the present disclosure, including, one or more chemotherapeutic agents. Such kits can also include, for example, other compounds and/or compositions, a device(s) for administering the compounds and/or compositions, and written instructions in a form prescribed by a governmental agency regulating the manufacture, use or sale of pharmaceuticals or biological products. The compositions described herein can be packaged as a single dose or for continuous or periodic discontinuous administration. For continuous administration, a package or kit can include the conjugates in each dosage unit (e.g., solution or other unit described above or utilized in oligonucleotide delivery), and optionally instructions for administering the doses daily, weekly, or monthly, for a predetermined length of time or as prescribed. If varying concentrations of a composition, of the components of the composition, or the relative ratios of the conjugates or agents within a composition over time is desired, a package or kit may contain a sequence of dosage units which provide the desired variability.

A number of packages or kits are known in the art for dispensing pharmaceutical agents for periodic oral use. In some embodiments, the package has indicators for each period. In some embodiments, the package is a labeled blister package, dial dispenser package, or bottle. The packaging means of a kit may itself be geared for administration, such as a syringe, pipette, eye dropper, or other such apparatus, from which the formulation may be applied to an affected area of the body, injected into a subject, or even applied to and mixed with the other components of the kit.

### Method of Treatment of Disease

The targeting moiety-linker-polymer-oligonucleotide conjugate provided herein can be used in methods of treating animals (for example mammals, such as humans and includes males, females, infants, children and adults).

In some embodiments, the conjugates provided herein may be used in a method of treating animals which comprises administering to the animal the conjugate of the present disclosure. The conjugates provided herein can be used as oligonucleotide carriers and oligonucleotide carrier components, in systems of controlled oligonucleotide release, preparations for low-invasive surgical procedures, etc. Pharmaceutical formulations can be injectable, implantable, etc.

In yet another aspect, the present disclosure provides a method of treating a disease or disorder in a subject in need thereof, comprising administering to the subject an efficient amount of at least one conjugate provided herein, wherein said conjugate releases one or more oligonucleotides upon biodegradation.

In some embodiments, the conjugates provided herein can be administered in vitro, in vivo and/or ex vivo to treat subjects and/or to modulate the growth of selected cell populations including, for example, cancer. In some embodiments, the particular types of cancers that can be treated with the conjugates provided herein include, but are not limited to: (1) solid tumors, including but not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor, cervical cancer, uterine cancer, testicular cancer, small cell lung carcinoma, non-small cell lung carcinoma, bladder carcinoma, lung cancer, epithelial carcinoma, glioma, glioblastoma, multiforme astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma; (2) blood-borne cancers, including but not limited to acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, multiple myeloma, acute and chronic leukemias, e.g., lymphoblastic myelogenous and lymphocytic myelocytic leukemias; and (3) lymphomas such as Hodgkin's disease, non-Hodgkin's Lymphoma, Multiple myeloma, Waldenstrom's macroglobulinemia, Heavy chain disease, and Polycythemia vera.

In some embodiments, the conjugates provided herein can be administered in vitro, in vivo and/or ex vivo to treat subjects and/or to modulate the growth of selected cell populations in patients having anal, astrocytoma, leukemia, lymphoma, head and neck, liver, testicular, cervical, sarcoma, hemangioma, esophageal, eye, laryngeal, mouth, mesothelioma, skin, myeloma, oral, rectal, throat, bladder, breast, uterus, ovary, prostate, lung, colon, pancreas, renal, or gastric cancer.

In certain embodiment, the cancers are selected from the group consisting of breast cancer, gastric cancer, non-small cell lung cancer (NSCLC), and ovarian cancer.

In some embodiment, the conjugates provided herein can be administered in vitro, in vivo and/or ex vivo to treat, prevent, reduce the risk of developing and/or delay onset of certain pathologies, for example, a cancer. For example, the conjugates provided herein are useful in treating, preventing, delaying the progression of or otherwise ameliorating a symptom of a cancer selected from the group consisting of anal cancer, astrocytoma, leukemia, lymphoma, head and neck cancer, liver cancer, testicular cancer, cervical cancer, sarcoma, hemangioma, esophageal cancer, eye cancer, laryngeal cancer, mouth cancer, mesothelioma, skin cancer, myeloma, oral cancer, rectal cancer, throat cancer, bladder cancer, breast cancer, uterine cancer, ovarian cancer, prostate cancer, lung cancer, non-small cell lung cancer (NSCLC), colon cancer, pancreatic cancer, renal cancer, and gastric cancer.

In some embodiments the conjugates provided herein can be administered in vitro, in vivo and/or ex vivo to treat autoimmune diseases, such as systemic lupus, rheumatoid arthritis, psoriasis, and multiple sclerosis; graft rejections, such as renal transplant rejection, liver transplant rejection, lung transplant rejection, cardiac transplant rejection, and bone marrow transplant rejection; graft versus host disease; viral infections, such as CMV infection, HIV infection, and AIDS; and parasite infections, such as giardiasis, amoebiasis, schistosomiasis, and the like.

In some embodiments, the conjugates provided herein can also be used for the manufacture of a medicament useful for treating or lessening the severity of disorders, such as, characterized by abnormal growth of cells (e.g., cancer).

In some embodiments, the oligonucleotide is locally delivered to a specific target cell, tissue, or organ.

In certain embodiments, the conjugates provided herein can further comprise or are associated with a diagnostic label. In certain embodiments, the diagnostic label is selected from the group consisting of: radiopharmaceutical or radioactive isotopes for gamma scintigraphy and PET, contrast agent for Magnetic Resonance Imaging (MRI), contrast agent for computed tomography, contrast agent for X-ray imaging method, agent for ultrasound diagnostic method, agent for neutron activation, moiety which can reflect, scatter or affect X-rays, ultrasounds, radiowaves and microwaves and fluorophores. In certain exemplary embodiments, the conjugate is further monitored in vivo.

Examples of diagnostic labels include, but are not limited to, diagnostic radiopharmaceutical or radioactive isotopes for gamma scintigraphy and PET, contrast agent for Magnetic Resonance Imaging (MRI) (for example paramagnetic atoms and superparamagnetic nanocrystals), contrast agent for computed tomography, contrast agent for X-ray imaging method, agent for ultrasound diagnostic method, agent for neutron activation, and moiety which can reflect, scatter or affect X-rays, ultrasounds, radiowaves and microwaves, fluorophores in various optical procedures, etc. Diagnostic radiopharmaceuticals include 7-emitting radionuclides, e.g., indium-111, technetium-99m and iodine-131, etc. Contrast agents for MRI (Magnetic Resonance Imaging) include magnetic compounds, e.g., paramagnetic ions, iron, manganese, gadolinium, lanthanides, organic paramagnetic moieties and superparamagnetic, ferromagnetic and antiferromagnetic compounds, e.g., iron oxide colloids, ferrite colloids, etc. Contrast agents for computed tomography and other X-ray based imaging methods include compounds absorbing X-rays, e.g., iodine, barium, etc. Contrast agents for ultrasound based methods include compounds which can absorb, reflect and scatter ultrasound waves, e.g., emulsions, crystals, gas bubbles, etc. Still other examples include substances useful for neutron activation, such as boron and gadolinium. Further, labels can be employed which can reflect, refract, scatter, or otherwise affect X-rays, ultrasound, radiowaves, microwaves and other rays useful in diagnostic procedures. Fluorescent labels can be used for photoimaging. In certain embodiments a modifier comprises a paramagnetic ion or group.

In another aspect, the present disclosure provides a method of treating a disease or disorder in a subject, comprising preparing an aqueous formulation of at least one conjugate provided herein and parenterally injecting said formulation in the subject.

In another aspect, the present disclosure provides a method of treating a disease or disorder in a subject, comprising preparing an implant comprising at least one conjugate provided herein, and implanting said implant into the subject. In certain embodiments, the implant is a biodegradable gel matrix.

In another aspect, the present disclosure provides a method for treating of an animal in need thereof, comprising administering a conjugate according to the methods described above.

In another aspect, the present disclosure provides a method for eliciting an immune response in an animal, comprising administering a conjugate as in the methods described above.

In another aspect, the present disclosure provides a method of diagnosing a disease in an animal, comprising steps of: administering a conjugate as in the methods described above, wherein said conjugate comprises a detectable molecule; and detecting the detectable molecule.

In some embodiments, the step of detecting the detectable molecule is performed non-invasively. In some embodiments, the step of detecting the detectable molecule is performed using suitable imaging equipment.

In some embodiments, a method for treating an animal comprises administering to the animal the conjugates provided herein as a packing for a surgical wound from which a tumor or growth has been removed. The conjugate packing will replace the tumor site during recovery and degrade and dissipate as the wound heals.

In certain embodiments, the conjugate provided herein is associated with a diagnostic label for in vivo monitoring.

The conjugates provided herein can be used for therapeutic, preventative, and analytical (diagnostic) treatment of animals. The conjugates are intended, generally, for parenteral administration, but in some cases may be administered by other routes.

In some embodiments, soluble or colloidal conjugates are administered intravenously. In some embodiments, soluble or colloidal conjugates are administered via local (e.g., subcutaneous, intramuscular) injection. In some embodiments, solid conjugates (e.g., particles, implants, oligonucleotide delivery systems) are administered via implantation or injection.

In some embodiments, conjugates comprising a detectable label are administered to study the patterns and dynamics of label distribution in animal body.

In certain embodiments, any one or more of the conjugates provided herein may be used in practicing any of the methods described above.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same.

### EXAMPLES

For the purpose of illustration, the following examples are included. The Examples provided herein describe the synthesis of the compounds and conjugates disclosed herein as well as intermediates used to prepare the compounds and conjugates. However, it is to be understood that these examples do not limit the present disclosure and are only meant to suggest a method of practicing the present disclosure. Persons skilled in the art will recognize that the chemical reactions described may be readily adapted to prepare a number of other compounds or conjugates of the present disclosure, and alternative methods for preparing the compounds and conjugates of the present disclosure are deemed to be within the scope of the present disclosure. Besides, persons skilled in the art will also understand that individual steps described herein or in the separate batches of a compound may be combined. Alternatively, other reactions disclosed herein or known in the art will be recognized as having applicability for preparing other compounds of the present disclosure. The following description is, therefore, not intended to limit the scope of the present disclosure, but rather is specified by the claims appended hereto.

### Example 1 Synthesis of the Copolymer Linker Compound 1

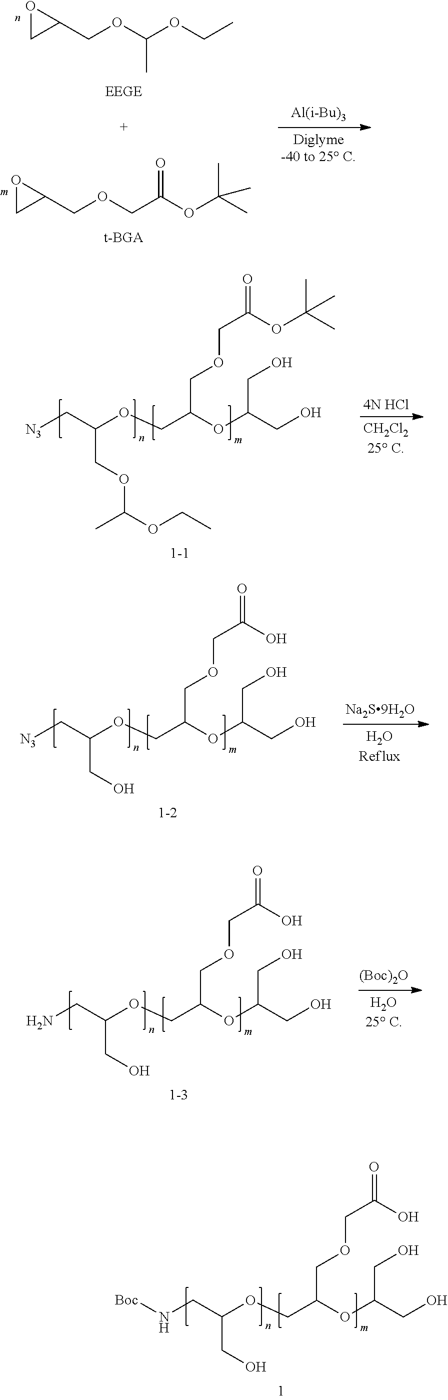

To the solution of N₃NBu₄ (1.98 g, 6.98 mmol) in diglyme (200 mL), EGE (61 g, 418.6 mmol) and t-BGA (39.3 g, 209.3 mmol) were added under N₂. The mixture was cooled to −40° C., then Al(iBu)₃ (30 ml, 29.7 mmol, 1M in hexanes) was added slowly. The reaction was warmed to room temperature and stirred under N₂ for 16 hours. The reaction was quenched with EtOH, diluted with water and extracted with EA. The combined organic phase was washed with brine, dried with Na₂SO₄, filtered and concentrated under reduced pressure to afford compound 1-1 (103 g) as colorless oil with Mw 27,000 which was determined by GPC with PS as standard.

To the solution of compound 1-1 (50 g, 1.85 mmol) in DCM (500 mL), HCl (200 mL, 1 M in 1,4-dioxane) was added slowly and the reaction mixture was stirred at room temperature for 16 hours. After completion, the solvent was removed under reduced pressure and the residue was dissolved with 10% NaOH solution. After adjusted the pH to 7 with 4 N HCl, the total volume of the solution was adjusted to 500 mL with water to obtain compound 1-2 as water solution.

To the solution of 1-2, Na₂S·9H₂O (30 g, 125 mmol) was added and reaction mixture was heated at 100° C. for 18 hours. After completion, the reaction was cooled to room temperature and the pH was adjust to 10 with 4 N HCl. The mixture was dialyzed with 3KD MWCO to afford compound 1-3 as water solution (300 mL).

To the solution of 1-3, NaOH (5 g, 125 mmol) and (Boc)₂O (30 g, 125 mmol) were added and the reaction mixture was stirred at room temperature for 18 hours. After completion, the mixture was dialyzed with 3KD MWCO to afford compound 1 as water solution (800 mL, 25 mg/mL).

1H NMR (400 MHz, D2O) δ 3.95 (s, 100H), 3.87-3.57 (br, 800H), 1.43 (s, 9H).

### Example 2 Synthesis of Compound 2

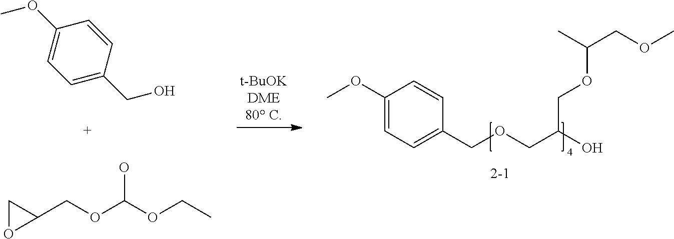

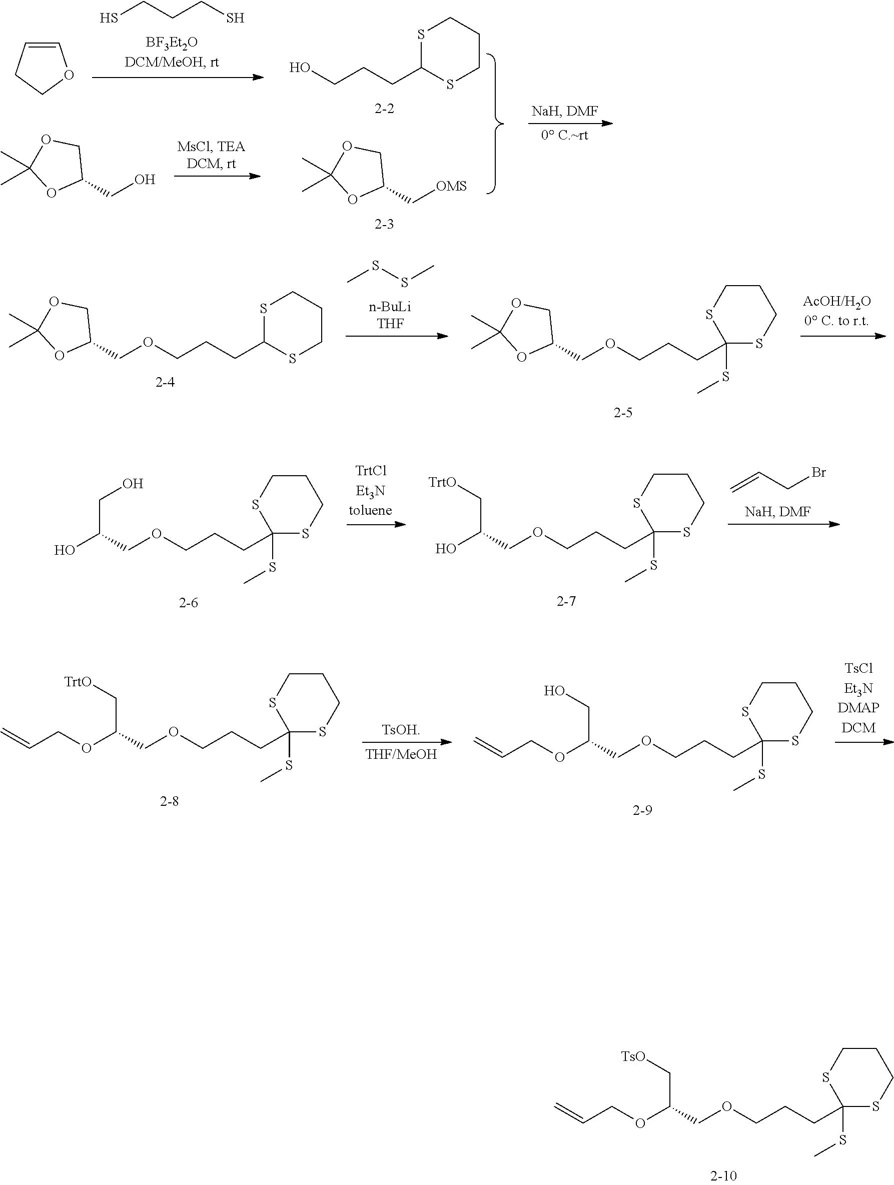

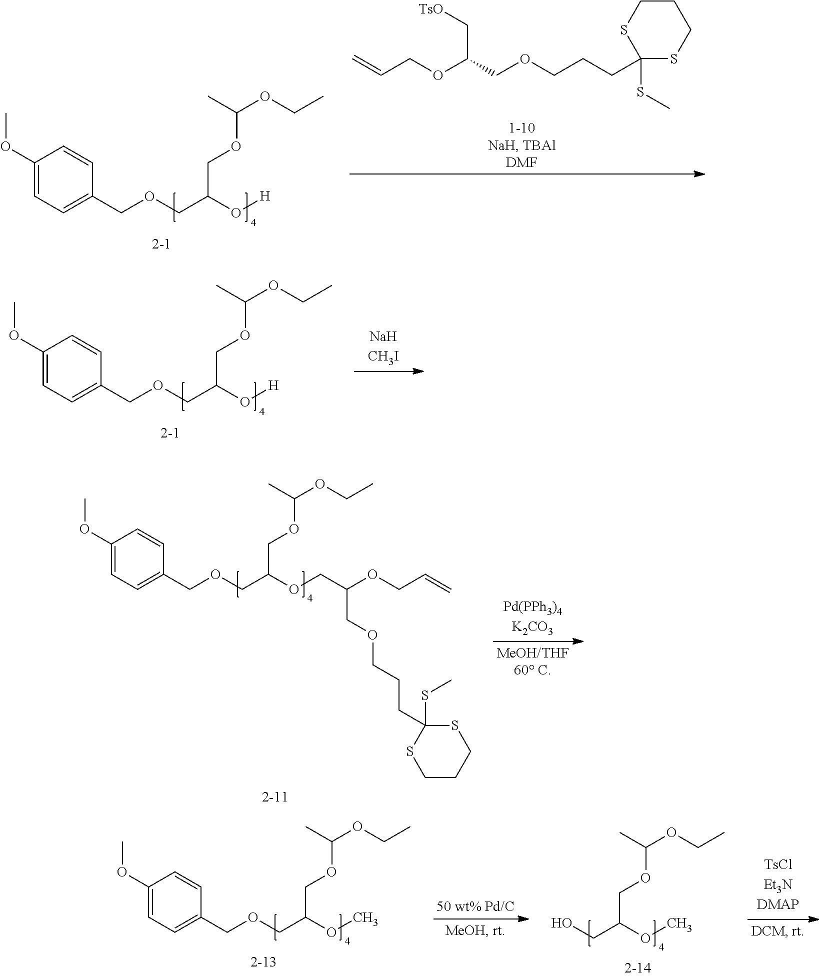

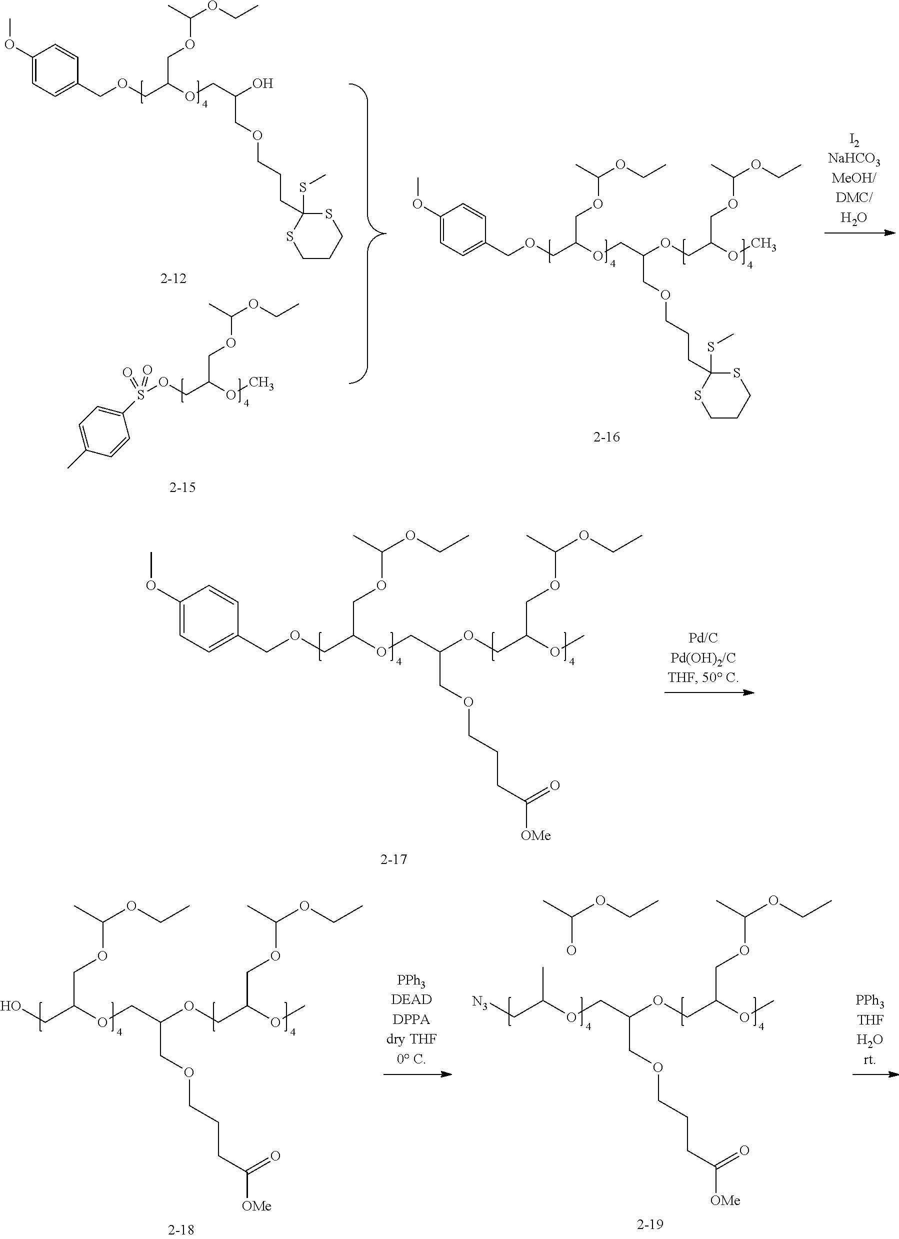

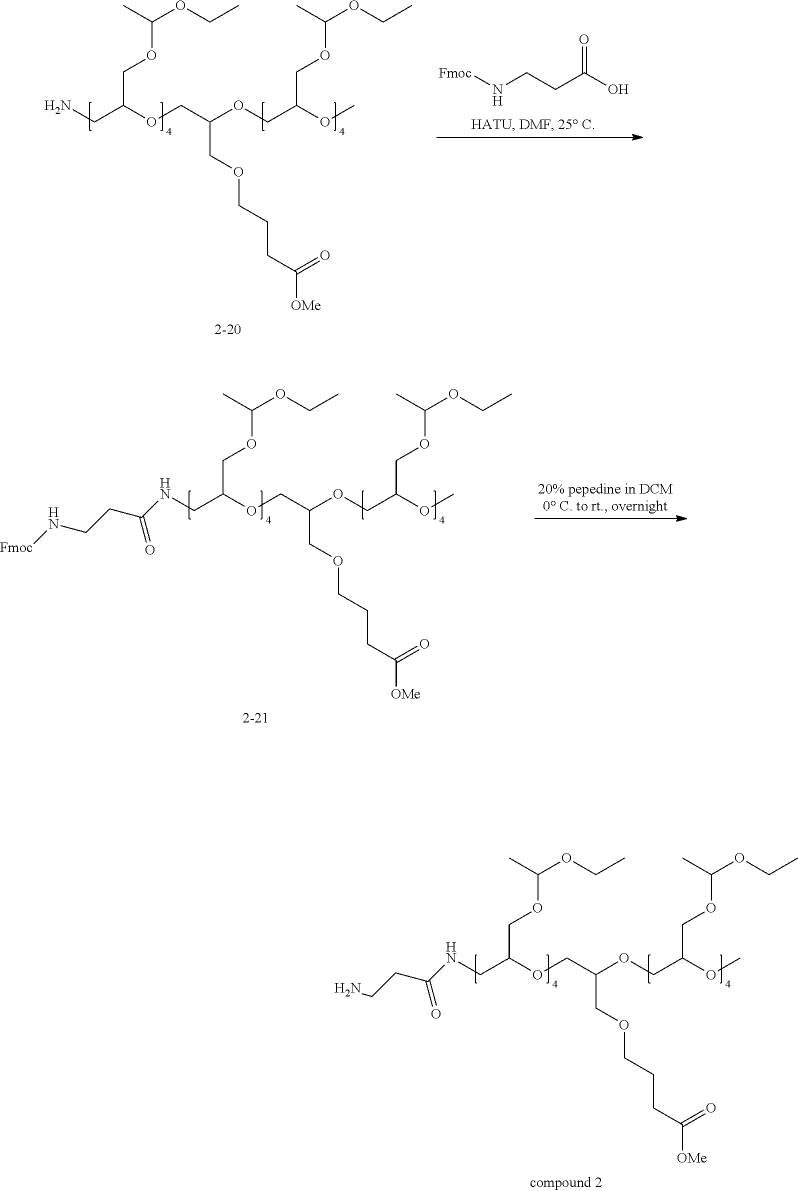

To a 250 mL flask was added 4-methoxybenzyl alcohol (5.0 g, 36.2 mmol), t-BuOK (7.3 mL, 7.2 mmol) and DME (25 mL) under N₂. The mixture was stirred at room temperature for 30 minutes, followed by addition of 2-((1-ethoxyethoxy) Methyl) oxirane (23.8 g, 16.9 mmol). The mixture was stirred at 80° C. for 16 hours. The reaction mixture was concentrated under reduced pressure and the residue was purified by reversed phase chromatography to afford Compound 2-1 (6.0 g, 8.3 mmol, 23%) as colorless oil.

To a solution of 2,3-dihydrofuran (90.0 g, 1.3 mol) in DCM (900 mL), MeOH (82.3 mL, 2.6 mol) and BF₃·Et₂O (2.5 mL, 19.5 mmol) was added at room temperature. The reaction mixture was stirred at room temperature for 10 minutes, followed by addition of 1,3-dimercaptopropane (138.5 g, 1.3 mol). The reaction mixture was stirred at room temperature for 2 hours, then diluted with DCM (1.5 L), washed with saturated sodium bicarbonate solution (500 mL) and water (500 mL×3). The combined organic layer was dried over anhydrous sodium sulfate and concentrated under reduced pressure, the residue was purified by flash chromatography on silica gel to afford Compound 2-2 (210.0 g, 1.2 mol, 92%) as a white solid.

To a solution of Compound 2-2 (210.0 g, 1.6 mol) in anhydrous DCM (2.0 L) was added Et₃N (241.3 g, 2.4 mmol) and MsCl (236.7 g, 2.1 mol) dropwise at 0° C. The mixture was stirred at room temperature for 2 hours. The reaction was quenched by addition of water (2 L) and extracted with DCM (1 L×3). The combined organic layer was washed with brine, dried over anhydrous sodium sulfate and concentrated under reduced pressure, the residue was purified by flash column chromatography on silica gel to Compound 2-3 (326.0 g, 1.6 mol, 98%) as light-yellow oil.

To a solution of Compound 2-2 (518.0 g, 2.9 mol) and Compound 2-3 (833.0 g, 4.0 mol) in anhydrous DMF (4 L) was added NaH (335 g, 8.4 mol) in portions at 0° C. under N₂. The reaction mixture was stirred at room temperature for 16 hours. The reaction was quenched by addition of water and extracted with EtOAc (1 L×3). The combined organic layer was washed with brine, dried over anhydrous sodium sulfate and concentrated under reduced pressure, the residue was purified by flash column chromatography on silica gel to afford Compound 2-4 (713.0 g, 2.4 mol, 84%) as light-yellow oil.

To a solution of Compound 2-4 (100.0 g, 342.0 mol) in anhydrous THF (800 mL) was added n-BuLi (274 mL, 2.5 M in hexane, 684.0 mmol) dropwise at −60° C. under N₂. The mixture was stirred at −60° C. for 40 minutes, followed by the addition of methyl disulfide (64.4 g, 684.0 mmol). The reaction mixture was stirred at −10° C. for 16 hours. The reaction was quenched with sat Nh₄Cl aq. and extracted with EtOAc (1 L×3). The combined organic layer was washed brine, dried over anhydrous sodium sulfate and concentrated under reduced pressure, the residue was purified by flash column chromatography on silica gel to afford Compound 2-5 (91.0 g, 269.0 mol, 78%) as light-yellow oil.

To a solution of Compound 2-5 (91.0 g, 269.0 mol) in H₂O (500 mL) was added AcOH (500 mL) dropwise at 0° C. The mixture was stirred at 0° C. for 4 hours. The reaction was quenched with sat. NahCO₃ aq. and extracted with DCM (1 L×3). The combined organic layer was dried over anhydrous sodium sulfate and concentrated under reduced pressure, the residue was purified by flash column chromatography on silica gel to afford Compound 2-6 (61.0 g, 204.4 mmol, 76%) as a light-yellow oil.

To a solution of Compound 2-6 (27.8 g, 93.2 mmol) in toluene (270 mL) was added TrtCl (26.0 g, 93.2 mmol) and Et₃N (18.9 g, 186.4 mmol). The reaction mixture was stirred at 110° C. for 3 hours. The mixture was concentrated under reduced pressure, and the residue was purified by flash column chromatography on silica gel to afford Compound 2-7 (43.8 g, 81.1 mmol, 87%) as colorless oil.

To a solution of Compound 2-7 (76.0 g, 140.5 mmol) in anhydrous DMF (800 mL) was added NaH (6.7 g, 168.6 mmol) at 0° C. under N₂. The mixture was stirred at 0° C. for 1 hour, followed by addition of allyl bromide (20.4 g, 168.6 mmol). The reaction mixture was stirred at room temperature for 3 hours. The reaction was quenched with water and extracted with EtOAc (1 L×3). The combined organic layers were washed brine, dried over anhydrous sodium sulfate and concentrated under reduced pressure to give a residue, which was purified by flash column chromatography on silica gel to afford the desired product Compound 2-8 (75.0 g, 129.1 mmol, 92%) as colorless oil.

To a solution of Compound 2-8 (75.0 g, 129.1 mmol) in anhydrous THF (85 mL) and anhydrous MeOH (750 mL) was added TsOH (4.4 g, 25.8 mmol) at room temperature under N₂. The mixture was stirred at room temperature for 2 hours. The reaction was quenched with sat NaHCO₃ solution and extracted with DCM (1 L×3). The combined organic layer was washed with brine, dried over anhydrous sodium sulfate and concentrated under reduced pressure to give a residue, which was purified by flash column chromatography on silica gel to afford the desired product give Compound 2-9 (35.4 g, 104.6 mmol, 81%) as colorless oil.

To a solution of Compound 2-9 (35.4 g, 104.6 mmol) in anhydrous DCM (350 mL) was added TsCl (21.2 g, 209.2 mmol), Et₃N (21.2 g, 209.2 mmol) and DMAP (1.27 g, 10.5 mmol) at room temperature under N₂. The mixture was stirred at room temperature for 4 hours. The reaction was quenched with water and extracted with DCM (500 mL×3). The combined organic layer was washed with brine, dried over anhydrous sodium sulfate and concentrated under reduced pressure to give a residue, which was purified by flash column chromatography on silica gel to afford the desired product Compound 2-10 (47.7 g, 96.8 mmol, 90%) as light-yellow oil.

To a solution of Compound 2-1 (4.4 g, 8.4 mmol) in DMF (30 mL), NaH (1.0 g, 24.5 mmol) was added at room temperature under N₂. After the mixture was stirred for 1 hour, TBAI (0.5 g, 1.2 mmol) and Compound 2-10 (6.0 g, 12.3 mmol) were added to the solution. The reaction mixture was heated to 90° C. and stirred for 16 hours. The reaction was quenched with water at 0° C. and extracted with EtOAc. The combined organic layers were washed with brine, dried over Na₂SO₄, filtered and concentrated under vacuum to give the crude, which was purified by silica gel chromatography to give Compound 2-11 (7.5 g, 7.2 mmol, 85%) as yellow oil.

Compound 2-11 (7.5 g, 7.2 mmol), Pd(PPh₃)₄(4.2 g, 3.6 mmol) and K₂CO₃(3.0 g, 21.6 mmol) were dissolved in MeOH/THF (55 mL, 6:1 v/v). The reaction was stirred at 60° C. for 16 hours under N₂. The mixture was concentrated under vacuum to give the crude, which was purified by silica gel chromatography to give Compound 2-12 (5.4 g, 5.3 mmol, 74%) as colorless oil.

To a solution of Compound 2-1 (20.0 g, 27.7 mmol) in anhydrous DMF (150 mL) was added NaH (2.2 g, 55.3 mmol) at room temperature under N₂. The mixture was stirred at room temperature for 1 hour, then iodomethane (3.5 mL, 55.3 mmol) was added. The reaction mixture was stirred at room temperature for 2 hours under N₂. The mixture was concentrated to give a residue, which was purified by reversed phase chromatography to afford the desired product Compound 2-13 (20.2 g, 27.4 mmol, 98%) as a white solid.

To a solution of Compound 2-13 (20.2 g, 27.4 mmol) in MeOH (200 mL) was added 10% Pd/C (10.0 g) and 10% Pd(OH)/C (200 mg) at room temperature. The reaction mixture was stirred at 40° C. for 16 hours under H₂. The mixture was filtered and concentrated to give the desired product Compound 2-14 (16.3 g, 26.4 mmol, 96%) as colorless oil.

To a solution of Compound 2-14 (16.3 g, 26.4 mmol) in anhydrous DCM (6 mL) was added Et₃N (15.0 mL, 105.7 mmol), DMAP (323.0 mg, 2.6 mmol) and TsCl (10.1 g, 52.9 mmol) at room temperature under N₂. The mixture was stirred at room temperature for 16 hours. The mixture was concentrated under reduced pressure to give a residue, which was purified by silica gel chromatography to afford the desired product Compound 2-15 (17.8 g, 23.1 mmol, 87% yield) as a light-yellow solid.

To a solution of Compound 2-12 (5.4 g, 5.4 mmol) in NMP (50 mL), NaH (0.9 g, 21.4 mmol) was added at room temperature under N₂. After the mixture was stirred for one hour, TBAI (400 mg, 1.07 mmol) and Compound 2-15 (8.2 g, 10.7 mmol) were added the solution. The reaction mixture was heated to 90° C. and stirred for 16 hours. The reaction was quenched with water at 0° C., and extracted with EtOAc. The combined organic layer was washed with brine, dried over Na₂SO₄, filtered, concentrated under vacuum to give the crude, which was purified by silica gel chromatography to give Compound 12-16 (7.1 g, 4.4 mmol, 83%) as yellow oil.

To a solution of Compound 2-16 (7.1 g, 4.4 mmol) and NaHCO₃ (3.4 g, 39.9 mmol) in MeOH/DCM/H₂O (300 mL, 40:20:1 v/v/v), 12 (3.4 g, 13.3 mmol) was added slowly at 0° C. The reaction was stirred at 0° C. for 10 min. The mixture was added NaHCO₃ (50 mL), and quenched with Na₂S₂O₃. The reaction was added water and extracted with DCM. The combined organic layer was washed with brine, dried over Na₂SO₄, filtered, concentrated under vacuum to give the crude, which was purified by silica gel column chromatography to give Compound 2-17 (5.3 g, 3.5 mmol, 80%) as yellow oil.

To a solution of Compound 2-17 (5.3 g, 3.5 mmol) in THF (200 mL), 10% Pd/C (3.7 g) and 10% Pd (OH)₂/C (3.7 g) were added under H₂. The reaction was stirred at 50° C. for 20 hours. The mixture was filtered, and concentrated under vacuum to give the crude, which was purified by silica gel chromatography to give Compound 2-18 (3.4 g, 2.4 mmol, 70%) as yellow oil.

To a solution of Compound 2-18 (3.4 g, 2.4 mmol) in THF (55 mL), PPh₃ (1.9 g, 7.3 mmol), DEAD (1.3 g, 7.3 mmol) and DPPA (2.4 g, 8.5 mmol) were added at 0° C. The reaction was stirred at 0° C. for 2 hours. After completion, the solvent was removed under reduced pressure and the residue was purified by silica gel column chromatography to give Compound 2-19 (3.3 g, 2.3 mmol, 96%) as yellow oil.

To a solution of Compound 2-19 (3.3 g, 2.3 mmol) in THF/H₂O (30 mL, 2:1 v/v), PPh₃ (1.3 g, 4.8 mmol) was added. The reaction was stirred at room temperature for 15 hours. The solvent was removed under reduced pressure and the residue was purified by silica gel column chromatography to give Compound 2-20 (2.3 g, 1.7 mmol, 71%) as colorless oil.

To a solution of Fmoc-alanine (500 mg, 1.7 mmol) in DMF (20 mL), DIPEA (0.6 g, 5.0 mmol), Compound 2-20 (2.3 g, 1.7 mmol) and HATU (1.0 g, 2.5 mmol) were added under N₂. The reaction was stirred at room temperature for 16 hours. The reaction was purified by flash chromatography on silica gel to give Compound 2-21 (2.7 g, 1.6 mmol, 97%) as colorless oil.

To a solution of Compound 2-21 (2.7 g, 1.6 mmol) in DCM (25 mL), piperidine (5 mL) was added slowly at 0° C. The reaction mixture was warmed to room temperature and stirred for 16 hours. Water (50 mL) was poured into the reaction and the reaction was extracted with DCM (50 mL×3). The combined organic layer was washed with brine, dried over Na₂SO₄, filtered, concentrated under vacuum to give the crude, which was purified by silica gel chromatography to give Compound 2 (2.1 g, 1.4 mmol, 87%) as colorless oil.

### Example 3 Synthesis of Compound 3

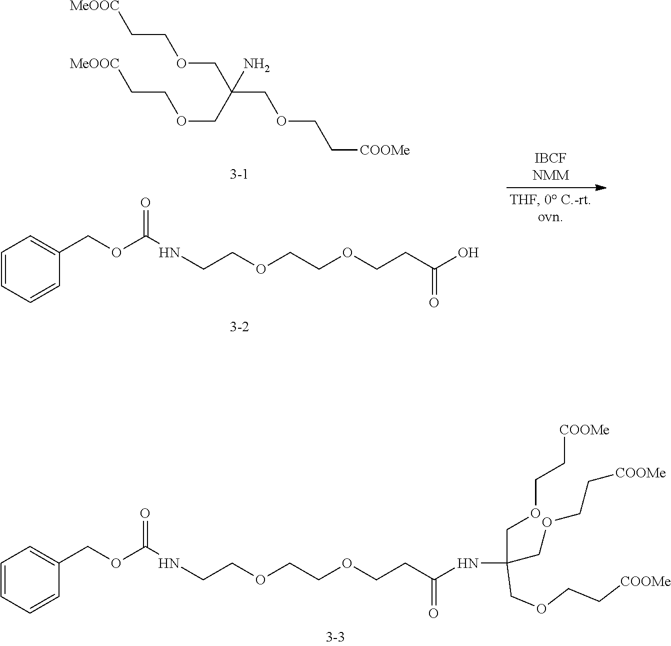 [SMILES: COC(=O)CCOCC(N)(COCCC(=O)OC)COCCC(=O)OC ; O=C(O)CCOCCOCCNC(=O)OCc1ccccc1 ; COC(=O)CCOCC(COCCC(=O)OC)(COCCC(=O)OC)NC(=O)CCOCCOCCNC(=O)OCc1ccccc1]

To a solution of 3-1 (2.9 g, 7.4 mmol), 3-2 (2.5 g, 6.7 mmol) and NMM (1.3 mL, 11.3 mmol) in DCM (60 mL), IBCF (1.2 mL, 9.4 mmol) was added slowly at 0° C. The reaction mixture was stirred at room temperature for 16 hours under N₂. The mixture was concentrated to give a residue. The residue was diluted with EtOAc (150 mL) and water (150 mL), and the aqueous layer was extracted with EtOAc (100 mL×3). The combined organic layer was wash with 0.5 M citric acid, sat. NaHCO₃(aq) and brine, dried over anhydrous sodium sulfate and concentrated under reduced pressure to give the crude, which was purified by silica gel chromatography to afford Compound 3-3 (3.3 g, 4.3 mmol, 64%) as light-yellow oil.

To a mixture of Compound 3-3 (7.4 g, 9.7 mmol) in MeOH (50 mL)/H₂O (50 mL) was added NaOH (1.2 g, 29.0 mmol) at room temperature. The reaction was stirred for 16 hours at room temperature. The pH was adjusted to 3 with 0.5 N HCl. The aqueous layer was extracted with DCM (200 mL×3). The combined organic layer was dried over anhydrous sodium sulfate and concentrated under reduced pressure to give the crude, which was purified by silica gel chromatography to afford Compound 3-4 (6.5 g, 9.0 mmol, 93%) as colorless solid.

To a solution of Compound 3-4 (2.1 g, 2.9 mmol) in DCM (18 mL)/DMF (2 mL), amine (2.9 g, 7.4 mmol), DIPEA (2.9 mL, 17.5 mmol), EDCI (1.9 g, 9.6 mmol) and HOAT (1.3 g, 9.6 mmol) were added at room temperature under N₂. The mixture was stirred for 5 minutes, followed by addition methyl 3-amino-2-hydroxypropanoate hydrochloride (1.4 g, 10.2 mmol). The reaction mixture was stirred at room temperature for 16 hours under N₂. The mixture was concentrated under reduced pressure, the residue was purified by C18 chromatography to afford Compound 3-5 (2.6 g, 2.6 mmol, 90%) as colorless oil.

To a solution of Compound 3-5 (2.6 g, 2.6 mmol) in MeOH (35 mL) was added 10% Pd/C (1.3 g), (Boc)₂O (1.7 g, 7.9 mmol) at room temperature. The reaction mixture was stirred at 35° C. for 16 hours under H₂. The mixture was filtered and the filtration was concentrated and the residue was purified by silica gel chromatography to afford Compound 3-6 (2.1 g, 2.2 mmol, 85%) as colorless oil.

To a mixture of Compound 3-6 (2.1 g, 2.2 mmol) in MeOH (15 mL) and H₂O (15 mL) was added NaOH (400 mg, 10 mmol) at room temperature. The reaction was stirred for 16 hours at room temperature. The pH was adjusted to around 3 by addition of critic acid. The aqueous layer was extracted with DCM (200 mL×3). The combined organic layer was dried over anhydrous sodium sulfate and concentrated under reduced pressure to give the desired product Compound 3-7 (1.7 g, 1.9 mmol, 83%) as colorless solid.

To a solution of Compound 3-7 (150.0 mg, 0.2 mmol) in DCM, DIPEA (180.0 uL, 1.0 mmol), EDC (140.0 uL, 1.0 mmol), HOBt (100.0 mg, 1.0 mmol) and Compound 2 (1.2 g, 1.0 mmol) were added sequentially. The reaction mixture was stirred at room temperature for 16 hours under N₂. Water was poured into the reaction and the reaction was extracted with DCM. The combined organic layer was washed with brine, dried over Na₂SO₄, filtered, concentrated under vacuum to give the crude, which was treated with lithium hydroxide in THF/water. Saturated NH₄Cl solution was poured into the reaction and the reaction was extracted with DCM. The combined organic layer was washed with brine, dried over Na₂SO₄, filtered, concentrated under vacuum to give Compound 3 (1.08 g, 75%) as colorless oil.

### Example 4 Synthesis of Compound 4

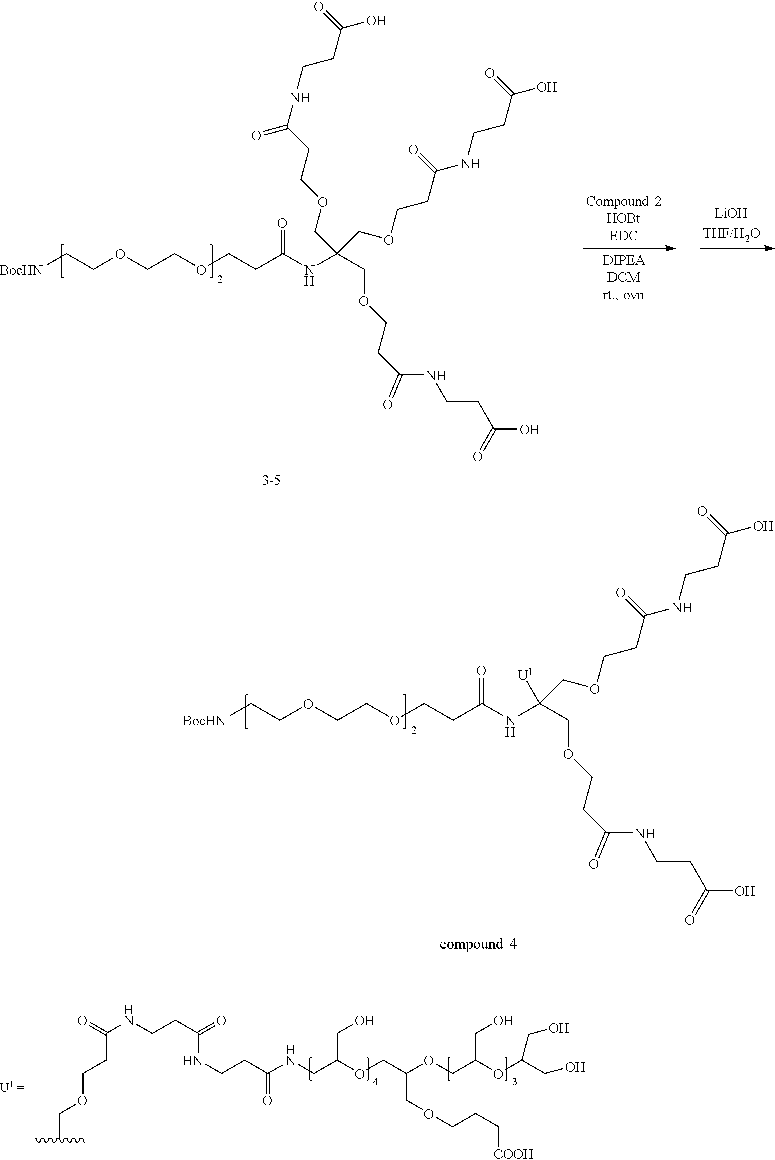

To a solution of Compound 3-5 (150.0 mg, 0.2 mmol) in DCM, DIPEA (36.0 uL, 0.2 mmol), EDC (28.0 uL, 0.2 mmol), HOBt (20.0 mg, 0.2 mmol), and Compound 1 (300 mg, 0.2 mmol) were added sequentially. The reaction mixture was stirred at room temperature for 16 hours under N₂. Water was added into the reaction and the reaction was extracted with DCM. The combined organic layer was washed with brine, dried over Na₂SO₄, filtered, concentrated under vacuum to give the crude, which was treated with lithium hydroxide in THF/water. Saturated NH₄Cl solution was added into the reaction and the reaction was extracted with DCM. The combined organic layers were washed with brine, dried over Na₂SO₄, filtered, concentrated under vacuum to give Compound 4 (148.0 mg, 50%) as colorless oil.

### Example 5 siRNA Targeting TGFβ

Unmodified siRNAs were designed to form duplex with TGFβ and thus block translation via steric blockage mechanism. The RNAs were synthesized using solid phase using standard phosphoramidite chemistry and purified over HPLC. Purified single strands were duplexed to get the double stranded siRNA.

Table 1 below illustrates the siRNA sequences designed and synthesized targeting TGFβ3, wherein siRNA_# comprises sense strand SS # and antisense strand AS #.

| | | | SEQ | | | SEQ |
|---|---|---|---|---|---|---|
| siRNA_# | SS No. | Sequence (5′-3′) | ID NO. | AS No. | Sequence (5′-3′) | ID NO. |
| 1 | SS1 | CACACUGCAAGUGGACAUCAA | 1 | AS1 | GAUGUCCACUUGCAGUGUGUU | 101 |
| 2 | SS2 | CACAUCAGAGCUCCGAGAAGC | 2 | AS2 | UUCUCGGAGCUCUGAUGUGUU | 102 |
| 3 | SS3 | CACUACUGUAGUUAGAUCUAU | 3 | AS3 | AGAUCUAACUACAGUAGUGUU | 103 |
| 4 | SS4 | GCCUGUCUGCACUAUUCCUUU | 4 | AS4 | AGGAAUAGUGCAGACAGGCAG | 104 |
| 5 | SS5 | GAGACGGAUCUCUCUCCGACC | 5 | AS5 | UCGGAGAGAGAUCCGUCUCCU | 105 |
| 6 | SS6 | GGCCCUGUACAACCAGCAUAA | 6 | AS6 | AUGCUGGUUGUACAGGGCCAG | 106 |
| 7 | SS7 | GAACACUACUGUAGUUAGAUC | 7 | AS7 | UCUAACUACAGUAGUGUUCCC | 107 |
| 8 | SS8 | GCCCAUCUAGGUUAUUUCCGU | 8 | AS8 | GGAAAUAACCUAGAUGGGCGC | 108 |
| 9 | SS9 | GGACACCAACUAUUGCUUCAG | 9 | AS9 | GAAGCAAUAGUUGGUGUCCAG | 109 |
| 10 | SS10 | GCGGUACCUGAACCCGUGUUG | 10 | AS10 | ACACGGGUUCAGGUACCGCUU | 110 |
| 11 | SS11 | GCCCCUACAUUUGGAGCCUGG | 11 | AS11 | AGGCUCCAAAUGUAGGGGCAG | 111 |
| 12 | SS12 | GCAAGACUAUCGACAUGGAGC | 12 | AS12 | UCCAUGUCGAUAGUCUUGCAG | 112 |
| 13 | SS13 | GACUCGCCAGAGUGGUUAUCU | 13 | AS13 | AUAACCACUCUGGCGAGUCGC | 113 |
| 14 | SS14 | GCCACAGAUCCCCUAUUCAAG | 14 | AS14 | UGAAUAGGGGAUCUGUGGCAG | 114 |
| 15 | SS15 | GGUGGAAACCCACAACGAAAU | 15 | AS15 | UUCGUUGUGGGUUUCCACCAU | 115 |
| 16 | SS16 | GCCGUGGAGGGGAAAUUGAGG | 16 | AS16 | UCAAUUUCCCCUCCACGGCUC | 116 |
| 17 | SS17 | GCCCCAUUAAAGAUGGAGAGA | 17 | AS17 | UCUCCAUCUUUAAUGGGGCCC | 117 |
| 18 | SS18 | GUGGGGAACACUACUGUAGUU | 18 | AS18 | CUACAGUAGUGUUCCCCACUG | 118 |
| 19 | SS19 | GCAGCUGUCCAACAUGAUCGU | 19 | AS19 | GAUCAUGUUGGACAGCUGCUC | 119 |
| 20 | SS20 | GCCCUGUACAACCAGCAUAAC | 20 | AS20 | UAUGCUGGUUGUACAGGGCCA | 120 |
| 21 | SS21 | GAGAGGACUGCGGAUCUCUGU | 21 | AS21 | AGAGAUCCGCAGUCCUCUCUC | 121 |
| 22 | SS22 | GGGAACACUACUGUAGUUAGA | 22 | AS22 | UAACUACAGUAGUGUUCCCCA | 122 |
| 23 | SS23 | GUGGAAACCCACAACGAAAUC | 23 | AS23 | UUUCGUUGUGGGUUUCCACCA | 123 |
| 24 | SS24 | GCAACACUCUGAGAUGCAGGG | 24 | AS24 | CUGCAUCUCAGAGUGUUGCUA | 124 |
| 25 | SS25 | CGGCAGCUGUACAUUGACUUC | 25 | AS25 | AGUCAAUGUACAGCUGCCGCA | 125 |
| 26 | SS26 | GACACCAACUAUUGCUUCAGC | 26 | AS26 | UGAAGCAAUAGUUGGUGUCCA | 126 |
| 27 | SS27 | GCAGUACAGCAAGGUCCUGGC | 27 | AS27 | CAGGACCUUGCUGUACUGCGU | 127 |
| 28 | SS28 | GCUGUCCAACAUGAUCGUGCG | 28 | AS28 | CACGAUCAUGUUGGACAGCUG | 128 |
| 29 | SS29 | CCAUAGCAACACUCUGAGAUG | 29 | AS29 | UCUCAGAGUGUUGCUAUGGUG | 129 |
| 30 | SS30 | GUUGUGCGGCAGUGGUUGAGC | 30 | AS30 | UCAACCACUGCCGCACAACUC | 130 |
| 31 | SS31 | GACCUGGCCACCAUUCAUGGC | 31 | AS31 | CAUGAAUGGUGGCCAGGUCAC | 131 |
| 32 | SS32 | GCAGAGUACACACAGCAUAUA | 32 | AS32 | UAUGCUGUGUGUACUCUGCUU | 132 |
| 33 | SS33 | GCGGCAGCUGUACAUUGACUU | 33 | AS33 | GUCAAUGUACAGCUGCCGCAC | 133 |
| 34 | SS34 | GUCUGCACUAUUCCUUUGCCC | 34 | AS34 | GCAAAGGAAUAGUGCAGACAG | 134 |
| 35 | SS35 | GAGGUUAAGGGAGGAGUUCCU | 35 | AS35 | GAACUCCUCCCUUAACCUCUC | 135 |
| 36 | SS36 | GCAUCCUAGACCCUUUCUCCU | 36 | AS36 | GAGAAAGGGUCUAGGAUGCGC | 136 |
| 37 | SS37 | GCAACAAUUCCUGGCGAUACC | 37 | AS37 | UAUCGCCAGGAAUUGUUGCUG | 137 |
| 38 | SS38 | GCAGGGAUAACACACUGCAAG | 38 | AS38 | UGCAGUGUGUUAUCCCUGCUG | 138 |
| 39 | SS39 | GCACUGUUGAAGUGCCUUACA | 39 | AS39 | UAAGGCACUUCAACAGUGCCC | 139 |
| 40 | SS40 | CACAACGAAAUCUAUGACAAG | 40 | AS40 | UGUCAUAGAUUUCGUUGUGGG | 140 |
| 41 | SS41 | GACCUCAGCUUUCCCUCGAGG | 41 | AS41 | UCGAGGGAAAGCUGAGGUCCU | 141 |
| 42 | SS42 | GAGGACUGCGGAUCUCUGUGU | 42 | AS42 | ACAGAGAUCCGCAGUCCUCUC | 142 |
| 43 | SS43 | GAGUGGUUAUCUUUUGAUGUC | 43 | AS43 | CAUCAAAAGAUAACCACUCUG | 143 |
| 44 | SS44 | GGAACACUACUGUAGUUAGAU | 44 | AS44 | CUAACUACAGUAGUGUUCCCC | 144 |
| 45 | SS45 | GAUCGUGCGCUCCUGCAAGUG | 45 | AS45 | CUUGCAGGAGCGCACGAUCAU | 145 |
| 46 | SS46 | GCACCUUGGGCACUGUUGAAG | 46 | AS46 | UCAACAGUGCCCAAGGUGCUC | 146 |
| 47 | SS47 | GCUUCAGCUCCACGGAGAAGA | 47 | AS47 | UUCUCCGUGGAGCUGAAGCAA | 147 |
| 48 | SS48 | GCAGCUGUACAUUGACUUCCG | 48 | AS48 | GAAGUCAAUGUACAGCUGCCG | 148 |
| 49 | SS49 | GUUCUUCAACACAUCAGAGCU | 49 | AS49 | CUCUGAUGUGUUGAAGAACAU | 149 |
| 50 | SS50 | CUCCUCCAGGAGACGGAUCUC | 50 | AS50 | GAUCCGUCUCCUGGAGGAGAA | 150 |
| 51 | SS51 | CCGCGUGCUAAUGGUGGAAAC | 51 | AS51 | UUCCACCAUUAGCACGCGGGU | 151 |
| 52 | SS52 | CGCAUCCUAGACCCUUUCUCC | 52 | AS52 | AGAAAGGGUCUAGGAUGCGCG | 152 |
| 53 | SS53 | CUGGCCACCAUUCAUGGCAUG | 53 | AS53 | UGCCAUGAAUGGUGGCCAGGU | 153 |
| 54 | SS54 | CUCCAGGAGACGGAUCUCUCU | 54 | AS54 | AGAGAUCCGUCUCCUGGAGGA | 154 |
| 55 | SS55 | CCACAGAUCCCCUAUUCAAGA | 55 | AS55 | UUGAAUAGGGGAUCUGUGGCA | 155 |
| 56 | SS56 | CCAGGAGACGGAUCUCUCUCC | 56 | AS56 | AGAGAGAUCCGUCUCCUGGAG | 156 |
| 57 | SS57 | CCUGGCGAUACCUCAGCAACC | 57 | AS57 | UUGCUGAGGUAUCGCCAGGAA | 157 |
| 58 | SS58 | GGCUGUAUUUAAGGACACCCG | 58 | AS58 | GGUGUCCUUAAAUACAGCCCC | 158 |
| 59 | SS59 | GGUGAGGAAACAAGCCCAGAG | 59 | AS59 | CUGGGCUUGUUUCCUCACCUU | 159 |
| 60 | SS60 | GGAUAGUGAAGAAGACAAUAA | 60 | AS60 | AUUGUCUUCUUCACUAUCCCC | 160 |
| 61 | SS61 | GGAGAGAGGACUGCGGAUCUC | 61 | AS61 | GAUCCGCAGUCCUCUCUCCAU | 161 |
| 62 | SS62 | GGACUAUCCACCUGCAAGACU | 62 | AS62 | UCUUGCAGGUGGAUAGUCCCG | 162 |
| 63 | SS63 | GAGAGAGGACUGCGGAUCUCU | 63 | AS63 | AGAUCCGCAGUCCUCUCUCCA | 163 |
| 64 | SS64 | GAUGCAGGGACUCUGAUAACA | 64 | AS64 | UUAUCAGAGUCCCUGCAUCUC | 164 |
| 65 | SS65 | GACUAUCCACCUGCAAGACUA | 65 | AS65 | GUCUUGCAGGUGGAUAGUCCC | 165 |
| 66 | SS66 | GACACGCAGUACAGCAAGGUC | 66 | AS66 | CCUUGCUGUACUGCGUGUCCA | 166 |
| 67 | SS67 | GGGGCCCCAUUAAAGAUGGAG | 67 | AS67 | CCAUCUUUAAUGGGGCCCCAG | 167 |
| 68 | SS68 | GUGACAGCAGGGAUAACACAC | 68 | AS68 | GUGUUAUCCCUGCUGUCACAG | 168 |
| 69 | SS69 | GCAGGGACUCUGAUAACACCC | 69 | AS69 | GUGUUAUCAGAGUCCCUGCAU | 169 |
| 70 | SS70 | CGCGUGCUAAUGGUGGAAACC | 70 | AS70 | UUUCCACCAUUAGCACGCGGG | 170 |
| 71 | SS71 | GUGGAUCCACGAGCCCAAGGG | 71 | AS71 | CUUGGGCUCGUGGAUCCACUU | 171 |
| 72 | SS72 | GCGUGCUAAUGGUGGAAACCC | 72 | AS72 | GUUUCCACCAUUAGCACGCGG | 172 |
| 73 | SS73 | CUGAUAACACCCAUUUUAAAG | 73 | AS73 | UUAAAAUGGGUGUUAUCAGAG | 173 |
| 74 | SS74 | AACCCACAACGAAAUCUAUGA | 74 | AS74 | AUAGAUUUCGUUGUGGGUUUC | 174 |
| 75 | SS75 | CUACUGUAGUUAGAUCUAUUU | 75 | AS75 | AUAGAUCUAACUACAGUAGUG | 175 |
| 76 | SS76 | CCUGAACCCGUGUUGCUCUCC | 76 | AS76 | AGAGCAACACGGGUUCAGGUA | 176 |
| 77 | SS77 | CACCAUAGCAACACUCUGAGA | 77 | AS77 | UCAGAGUGUUGCUAUGGUGAC | 177 |
| 78 | SS78 | CACCCACCUUCUGGUACCAGA | 78 | AS78 | UGGUACCAGAAGGUGGGUGGU | 178 |
| 79 | SS79 | CACUGCAAGUGGACAUCAACG | 79 | AS79 | UUGAUGUCCACUUGCAGUGUG | 179 |
| 80 | SS80 | GCACUAUUCCUUUGCCCGGCA | 80 | AS80 | CCGGGCAAAGGAAUAGUGCAG | 180 |
| 81 | SS81 | CGAGAAGCGGUACCUGAACCC | 81 | AS81 | GUUCAGGUACCGCUUCUCGGA | 181 |
| 82 | SS82 | UGGCCACCAUUCAUGGCAUGA | 82 | AS82 | AUGCCAUGAAUGGUGGCCAGG | 182 |
| 83 | SS83 | UACCUGAACCCGUGUUGCUCU | 83 | AS83 | AGCAACACGGGUUCAGGUACC | 183 |
| 84 | SS84 | CCUCCCGCAAAGACUUUUCCC | 84 | AS84 | GAAAAGUCUUUGCGGGAGGCC | 184 |
| 85 | SS85 | CUGUUGAAGUGCCUUACAUUA | 85 | AS85 | AUGUAAGGCACUUCAACAGUG | 185 |
| 86 | SS86 | CACCAUUCAUGGCAUGAACCG | 86 | AS86 | GUUCAUGCCAUGAAUGGUGGC | 186 |
| 87 | SS87 | GUACAUUGACUUCCGCAAGGA | 87 | AS87 | CUUGCGGAAGUCAAUGUACAG | 187 |
| 88 | SS88 | UGACAGCAGGGAUAACACACU | 88 | AS88 | UGUGUUAUCCCUGCUGUCACA | 188 |
| 89 | SS89 | CCUGGACACCAACUAUUGCUU | 89 | AS89 | GCAAUAGUUGGUGUCCAGGGC | 189 |
| 90 | SS90 | GACUUCCGCAAGGACCUCGGC | 90 | AS90 | CGAGGUCCUUGCGGAAGUCAA | 190 |
| 91 | SS91 | CACGUGGAGCUGUACCAGAAA | 91 | AS91 | UCUGGUACAGCUCCACGUGCU | 191 |
| 92 | SS92 | CCAGAAAUACAGCAACAAUUC | 92 | AS92 | AUUGUUGCUGUAUUUCUGGUA | 192 |
| 93 | SS93 | CGCAGUACAGCAAGGUCCUGG | 93 | AS93 | AGGACCUUGCUGUACUGCGUG | 193 |
| 94 | SS94 | CCAUCUAGGUUAUUUCCGUGG | 94 | AS94 | ACGGAAAUAACCUAGAUGGGC | 194 |
| 95 | SS95 | CAGAGUGGUUAUCUUUUGAUG | 95 | AS95 | UCAAAAGAUAACCACUCUGGC | 195 |
| 96 | SS96 | CCUACCUUUUGCCGGGAGACC | 96 | AS96 | UCUCCCGGCAAAAGGUAGGAG | 196 |
| 97 | SS97 | CAGCAUAUAUAUGUUCUUCAA | 97 | AS97 | GAAGAACAUAUAUAUGCUGUG | 197 |
| 98 | SS98 | GCGGAUCUCUGUGUCAUUGGG | 98 | AS98 | CAAUGACACAGAGAUCCGCAG | 198 |
| 99 | SS99 | CCCCUACAUUUGGAGCCUGGA | 99 | AS99 | CAGGCUCCAAAUGUAGGGGCA | 199 |
| 100 | SS100 | CCCACAACGAAAUCUAUGACA | 100 | AS100 | UCAUAGAUUUCGUUGUGGGUU | 200 |

### Example 6 Silencing of TGFβ with the siRNA

### Cell Culture

Human pancreatic cells (BxPC-3 and PANC-1) were purchased from ATCC, BxPC-3 and PANC-1 were cultured in RPMI-1640 medium (ATCC 30-2001) with 10% fetal bovine serum (FBS) (Hyclone), 100 units/ml penicillin and 100 μg/ml streptomycin. Cells were incubated at 37° C. in 5% CO₂.

In Vitro Evaluation of siRNA or ASO Mediated Gene Silencing

Total siRNAs and antisense oligonucleotides (ASOs) targeting TGFβ1 were screened in human PANC-1 cells. Briefly, cells were seeded into 6-well plate at density 3×10⁵ cells per well with 2 mL culture medium, so that they would be at 60%~80% confluent at the time of transfection. Unless otherwise stated, all the reagents were used according to manufacturer's recommendations.

The transient transfection was performed using Lipofectamine™ 2000 (Invitrogen), with oligonucleotides' final concentration at 100 nM. After 48-hours incubation at 37° C., total RNAs were isolated with RNeasy Mini Kit (Qiagen). To generate cDNA, reverse-transcription was carried out using High Capacity cDNA Reverse Transcription kit (ABI).

Quantitative real-time PCR using TaqMan® Gene Expression Assays (ThermoFisher) was performed on the QuantStudio6 pro (ABI) to quantify the relative expression level of target gene, which was ultimately calculated by the 2-ΔΔCT algorithm. β-actin was chosen as the internal control. Potent oligonucleotides that could induce more than 80% silencing of TGFβ 1 mRNA were selected for subsequent tests.

Table 2 below illustrates the TGFβ silencing level for exemplary siRNA.

| | Silent efficiency at 100 | |
|---|---|---|
| | nM (%) | |
| oligo ID | PANC-1 | BxPC-3 |
| siRNA_1 | 49.93 | — |
| siRNA_2 | 55.80 | — |
| siRNA_3 | 125.90 | — |
| siRNA_4 | 34.13 | — |
| siRNA_5 | 14.15 | — |
| siRNA_6 | 37.86 | — |
| siRNA_7 | 34.78 | — |
| siRNA_8 | | — |
| siRNA_9 | | — |
| siRNA_10 | | — |
| siRNA_11 | 17.40 | — |
| siRNA_12 | | — |
| siRNA_13 | | — |
| siRNA_14 | | — |
| siRNA_15 | 11.83 | — |
| siRNA_16 | 96.85 | — |
| siRNA_17 | 6.73 | 6.20 |
| siRNA_18 | 113.19 | — |
| siRNA_19 | 21.14 | — |
| siRNA_20 | | — |
| siRNA_21 | | — |
| siRNA_22 | | — |
| siRNA_23 | 7.61 | 4.86 |
| siRNA_24 | 23.99 | — |
| siRNA_25 | 47.22 | — |
| siRNA_26 | 5.32 | 3.87 |
| siRNA_27 | 37.52 | — |
| siRNA_28 | 0.48 | 17.95 |
| siRNA_29 | 73.04 | — |
| siRNA_30 | 43.07 | — |
| siRNA_31 | 48.35 | — |
| siRNA_32 | 179.98 | — |
| siRNA_33 | 165.99 | — |
| siRNA_34 | 25.53 | — |
| siRNA_35 | 29.44 | — |
| siRNA_36 | 0.64 | 3.99 |
| siRNA_37 | 3.27 | 128.36 |
| siRNA_38 | 1.36 | 444.47 |
| siRNA_39 | 47.38 | — |
| siRNA_40 | 23.55 | — |
| siRNA_41 | 53.78 | — |
| siRNA_42 | 7.21 | 193.29 |
| siRNA_43 | 14.84 | — |
| siRNA_44 | | — |
| siRNA_45 | | — |
| siRNA_46 | | — |
| siRNA_47 | 1.82 | 1.70 |
| siRNA_48 | 2.13 | 1.76 |
| siRNA_49 | 31.97 | — |
| siRNA_50 | 35.50 | — |
| siRNA_51 | 14.14 | — |
| siRNA_52 | | — |
| siRNA_53 | | — |
| siRNA_54 | | — |
| siRNA_55 | 4.48 | 65.59 |
| siRNA_56 | 5.64 | 17.87 |
| siRNA_57 | 4.57 | 10.91 |
| siRNA_58 | 3.66 | 3.51 |
| siRNA_59 | 718.34 | — |
| siRNA_60 | 12.89 | — |
| siRNA_61 | 0.52 | 24.67 |
| siRNA_62 | 2.01 | 2.29 |
| siRNA_63 | 15.35 | — |
| siRNA_64 | | — |
| siRNA_65 | | — |
| siRNA_66 | | — |
| siRNA_67 | 46.57 | — |
| siRNA_68 | | — |
| siRNA_69 | | — |
| siRNA_70 | | — |
| siRNA_71 | 1.49 | 76.67 |
| siRNA_72 | 16.84 | — |
| siRNA_73 | 97.06 | 289.94 |
| siRNA_74 | 52.87 | 628.70 |
| siRNA_75 | 59.05 | — |
| siRNA_76 | | — |
| siRNA_77 | | — |
| siRNA_78 | | — |
| siRNA_79 | 60.05 | — |
| siRNA_80 | | — |
| siRNA_81 | | — |
| siRNA_82 | | — |
| siRNA_83 | 13.43 | — |
| siRNA_84 | | — |
| siRNA_85 | | — |
| siRNA_86 | | — |
| siRNA_87 | 42.07 | — |
| siRNA_88 | | — |
| siRNA_89 | | — |
| siRNA_90 | | — |
| siRNA_91 | 63.17 | — |
| siRNA_92 | | — |
| siRNA_93 | | — |
| siRNA_94 | | — |
| siRNA_95 | 117.14 | — |
| siRNA_96 | | — |
| siRNA_97 | | — |
| siRNA_98 | | — |
| siRNA_99 | 72.22 | — |
| siRNA_100 | 80.90 | — |

### Example 7 Modified siRNAs and Silencing Efficiency Thereof

The siRNAs showing good silencing ability were selected and redesigned to form modified siRNAs. The sequences were showed in table 3. The RNAs were synthesized using solid phase using modified phosphoramidite and purified over HPLC. Purified single strands were duplexed to get the double stranded siRNA. The silencing efficiency was tested with PANC-1 cell using the protocol as example 2.

Table 3 modified siRNAs and corresponding silencing efficiency

| | | | | Silencing |
|---|---|---|---|---|
| siRNA | | | SEQ | efficiency |
| No. | Sequence (5′-3′) | Strand | ID NO. | (%) |
| 17 | mG*mG*mGmCmCmC/i2FC/mA/i2FU//12FU//i2FA/ | SS | 201 | 26.17 |
| | mAmAmGmAmUmGmGmAmGmA | | | |
| | mU*/12FC/*mUmCmC/12FA//iGNA- | AS | 202 | |
| | T//12FC//i2FU/mUmUmAmA/i2FU/mG/i2FG/mGmG | | | |
| | mCmCmC*mC*mA | | | |
| 23 | mU*mG*mGmUmGmG/12FA/mA/i2FA//i2FC//i2FC/ | SS | 203 | 2.78 |
| | mCmAmCmAmAmCmGmAmAmA | | | |
| | mU*/12FU/*mUmCmG/i2FU//iGNA- | AS | 20 | |
| | T//12FG//12FU/mGmGmGmU/i2FU/mU/i2FC/mCmA | | | |
| | mCmCmA*mU*mU | | | |
| 26 | mU*mG*mGmAmCmA/i2FC/mC/i2FA//i2FA//12FC/ | SS | 20 | 7.12 |
| | mUmAmUmUmGmCmUmUmCmA | | | |
| | mU*/12FG/*mAmAmG/i2FC//iGNA- | AS | 206 | |
| | A//i2FA//12FU/mAmGmUmU/i2FG/mG/12FU/mGm | | | |
| | UmCmCmA*mG*mG | | | |
| 28 | mC*mA*mGmCmUmG/12FU/mC/12FC//12FA//12FA/ | SS | 20 | 29.92 |
| | mCmAmUmGmAmUmCmGmUmG | | | |
| | mC*/12FA/*mCmGmA/i2FU//iGNA- | AS | 20 | |
| | C//12FA//12FU/mGmUmUmG/i2FG/mA/i2FC/mAmG | | | |
| | mCmUmG*mC*mU | | | |
| 36 | mG*mC*mGmCmAmU/i2FC/mC/i2FU//12FA//i2FG/ | SS | 20 | 125.54 |
| | mAmCmCmCmUmUmUmCmUmC | | | |
| | mG*/i2FA/*mGmAmA/i2FA//iGNA- | AS | 210 | |
| | G//12FG//i2FG/mUmCmUmA/i2FG/mG/12FA/mUm | | | |
| | GmCmGmC*mG*mG | | | |
| 37 | mC*mA*mGmCmAmA/i2FC/mA/i2FA//i2FU//12FU/ | SS | 211 | 9.30 |
| | mCmCmUmGmGmCmGmAmUmA | | | |
| | mU*/12FA/*mUmCmG/i2FC//iGNA- | AS | 212 | |
| | C//12FA//i2FG/mGmAmAmU/i2FU/mG/i2FU/mUm | | | |
| | GmCmUmG*mU*mA | | | |
| 38 | mC*mA*mGmCmAmG/i2FG/mG/12FA//12FU//12FA/ | SS | 213 | 10.34 |
| | mAmCmAmCmAmCmUmGmCmA | | | |
| | mU*/12FG/*mCmAmG/12FU//iGNA- | AS | 214 | |
| | G//i2FU//12FG/mUmUmAmU/i2FC/mC/i2FC/mUmG | | | |
| | mCmUmG*mU*mC | | | |
| 42 | mG*mA*mGmAmGmG/12FA/mC/12FU//12FG//i2FC/ | SS | 215 | 40.30 |
| | mGmGmAmUmCmUmCmUmGmU | | | |
| | mA*/12FC/*mAmGmA/i2FG//iGNA- | AS | 216 | |
| | A//12FU//i2FC/mCmGmCmA/i2FG/mU/i2FC/mCmU | | | |
| | mCmUmC*mU*mC | | | |
| 47 | mU*mU*mGmCmUmU/i2FC/mA/i2FG//i2FC//12FU/ | SS | 217 | 15.69 |
| | mCmCmAmCmGmGmAmGmAmA | | | |
| | mU*/12FU/*mCmUmC/i2FC//iGNA- | AS | 218 | |
| | G//12FU//12FG/mGmAmGmC/12FU/mG/12FA/mAm | | | |
| | GmCmAmA*mU*mA | | | |
| 48 | mC*mG*mGmCmAmG/i2FC/mU/i2FG//12FU//i2FA/ | SS | 219 | 17.78 |
| | mCmAmUmUmGmAmCmUmUmC | | | |
| | mG*/12FA/*mAmGmU/i2FC//iGNA- | AS | 220 | |
| | A//12FA//12FU/mGmUmAmC/12FA/mG/i2FC/mUmG | | | |
| | mCmCmG*mC*mA | | | |
| 55 | mU*mG*mCmCmAmC/12FA/mG/12FA//12FU//i2FC/ | SS | 221 | 11.85 |
| | mCmCmCmUmAmUmUmCmAmA | | | |
| | mU*/12FU/*mGmAmA/i2FU//iGNA- | AS | 222 | |
| | A//12FG//i2FG/mGmGmAmU/i2FC/mU/i2FG/mUm | | | |
| | GmGmCmA*mG*mG | | | |
| 56 | mC*mU*mCmCmAmG/i2FG/mA/i2FG//i2FA//i2FC/ | SS | 223 | 14.16 |
| | mGmGmAmUmCmUmCmUmCmU | | | |
| | mA*/12FG/*mAmGmA/i2FG//iGNA- | AS | 224 | |
| | A//12FU//i2FC/mCmGmUmC/12FU/mC/i2FC/mUmG | | | |
| | mGmAmG*mG*mA | | | |
| 57 | mU*mU*mCmCmUmG/12FG/mC/12FG//i2FA//12FU/ | SS | 225 | 15.69 |
| | mAmCmCmUmCmAmGmCmAmA | | | |
| | mU*/12FU/*mGmCmU/i2FG//iGNA- | AS | 226 | |
| | A//12FG//i2FG/mUmAmUmC/i2FG/mC/i2FC/mAmG | | | |
| | mGmAmA*mU*mU | | | |
| 58 | mG*mG*mGmGmCmU/i2FG/mU/i2FA//12FU//12FU | SS | 227 | 12.57 |
| | /mUmAmAmGmGmAmCmAmCmC | | | |
| | mG*/12FG/*mUmGmU/i2FC//iGNA- | AS | 228 | |
| | C//12FU//i2FU/mAmAmAmU/i2FA/mC/i2FA/mGmC | | | |
| | mCmCmC*mC*mA | | | |
| 61 | mA*mU*mGmGmAmG/i2FA/mG/12FA//12FG//12FG | SS | 229 | 225.28 |
| | /mAmCmUmGmCmGmGmAmUmC | | | |
| | mG*/12FA/*mUmCmC/12FG//iGNA- | AS | 230 | |
| | C//12FA//i2FG/mUmCmCmU/i2FC/mU/i2FC/mUmC | | | |
| | mCmAmU*mC*mU | | | |
| 62 | mC*mG*mGmGmAmC/i2FU/mA/12FU//12FC//i2FC/ | SS | 231 | 25.04 |
| | mAmCmCmUmGmCmAmAmGmA | | | |
| | mU*/12FC/*mUmUmG/i2FC//iGNA- | AS | 232 | |
| | A//i2FG//i2FG/mUmGmGmA/i2FU/mA/i2FG/mUm | | | |
| | CmCmCmG*mC*mG | | | |
| 71 | mA*mA*mGmUmGmG/12FA/mU/i2FC//12FC//12FA/ | SS | 233 | 26.17 |
| | mCmGmAmGmCmCmCmAmAmG | | | |
| | mC*/i2FU/*mUmGmG/12FG//iGNA- | AS | 234 | |
| | C//12FU//i2FC/mGmUmGmG/12FA/mU/i2FC/mCmAmC | | | |
| | mUmU*mC*mC | | | |

wherein “m” represents 4′-OMe modified ribosyl sugar moiety, “*” represents a phosphonothioate internucleoside linkage, “i2F” represents 2′-F modified ribosyl sugar moiety, “iGNA” represents glycol nucleic acid (GNA) backbone, “/” and “//” are only used to clearly represent the sequences, without substantial meanings.

### Example 8 Unmodified ASOs and Silencing Efficiency Thereof

Unmodified ASOs were designed to form duplex with TGFβ and thus block translation via steric blockage mechanism. The RNAs were synthesized using solid phase using standard phosphoramidite chemistry and purified over HPLC. The silencing efficiency was tested with PANG-1 cell using the protocol as in Example 2.

TABLE 4
ASO sequences and silent efficiency thereof.

| | | silencing |
|---|---|---|
| SEQ ID | | efficiency at |
| NO. | ASO sequence¹ | 100 nM (%) |
| 235 | AGGGCGCGAAGGGCGGCGGCGGC | 31.12 |
| 236 | AGGGCGGcauGGGGGAG | 47.08 |
| 237 | GAGGGCGGcauGGGGGA | 76.32 |
| 238 | AGGGCGGcauGGGGGAGG | 31.19 |
| 239 | GAGGGCGGcauGGGGGAG | 20.14 |
| 240 | GGAGGGCGGcauGGGGGA | 37.84 |
| 241 | AGGGCGGcauGGGGGAGGC | 35.89 |
| 242 | GAGGGCGGcauGGGGGAGG | 22.94 |
| 243 | GGAGGGCGGcauGGGGGAG | 57.45 |
| 244 | CGGAGGGCGGcauGGGGGA | 25.83 |
| 245 | AGGGCGGcauGGGGGAGGCG | 21.92 |
| 246 | GAGGGCGGcauGGGGGAGGC | 22.96 |
| 247 | GGAGGGCGGcauGGGGGAGG | 21.29 |
| 248 | CGGAGGGCGGcauGGGGGAG | 7.38 |
| 249 | CCGGAGGGCGGcauGGGGGA | 35.28 |
| 250 | AGGGCGGcauGGGGGAGGCGG | 28.76 |
| 251 | GAGGGCGGcauGGGGGAGGCG | 63.57 |
| 252 | GGAGGGCGGcauGGGGGAGGC | 122.37 |
| 253 | CGGAGGGCGGcauGGGGGAGG | 108.21 |
| 254 | CCGGAGGGCGGcauGGGGGAG | 64.54 |
| 255 | CCCGGAGGGCGGcauGGGGGA | 55.47 |
| 256 | AGGGCGGcauGGGGGAGGCGGC | 47.47 |
| 257 | CCCGGAGGGCGGcauGGGGGAGG | 35.15 |

¹Initiation codon has been shown in bold.

### Example 9 PMOs and Silencing Efficiency Thereof

The ASO sequences showing good silencing ability were selected to make PMO showed in table 5. The PMOs were synthesized using solid phase using modified phosphoramidite and purified over HPLC. The silencing efficiency was tested with PANG-1 cell using the protocol as in Example 2.

TABLE 5
PMO sequence and silent efficiency

| | | Silent efficiency |
|---|---|---|
| SEQ | | at 100 nM |
| ID NO. | PMO sequence¹ | (%) |
| 258 | GAGGGCGGcauGGGGGAG | 24.93% |
| 259 | GAGGGCGGcauGGGGGAGG | 31.13% |
| 260 | AGGGCGGcauGGGGGAGGCG | 30.31% |
| 261 | GAGGGCGGcauGGGGGAGGC | 28.94% |
| 262 | GGAGGGCGGcauGGGGGAGG | 30.06% |
| 263 | CGGAGGGCGGcauGGGGGAG | 33.39% |

¹Initiation codon has been shown in bold.

### Example 10 siRNA Conjugation with αvβ6 Antibody

Certain modified siRNA sequence was selected for conjugation with antibody. For conjugation, conjugation moiety —NH—(CH₂)₆H— or —S—(CH₂)₆— was selected and installed on the passenger strand at 3′ end or 5′ end. Below is a representative structure of siRNA passenger with conjugation moiety —NH—(CH₂)₆— at 5′ or 3′ end.

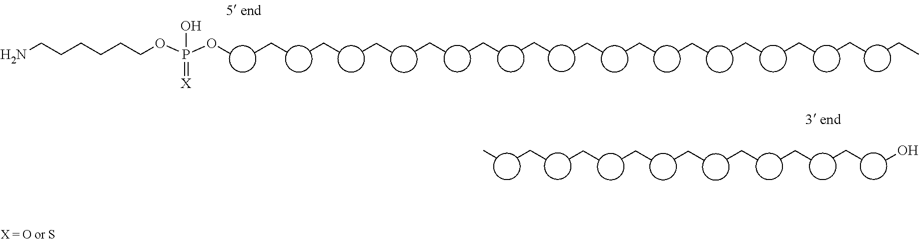

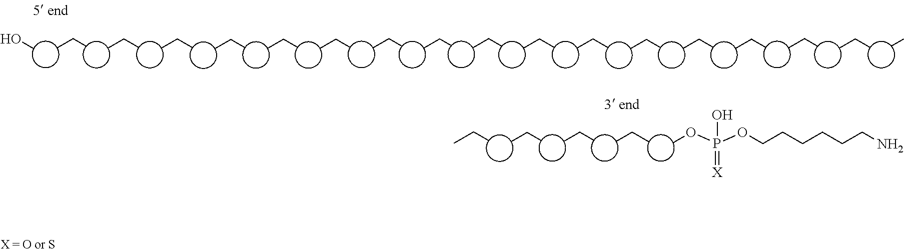

The conjugation moiety carrying the modified siRNA was coupled with the Linker-Polymer compound to form Linker-Polymer-Oligonucleotide compound, which could further conjugate with antibody. Below is a representative structure of Linker-Polymer-Oligonucleotide compound.

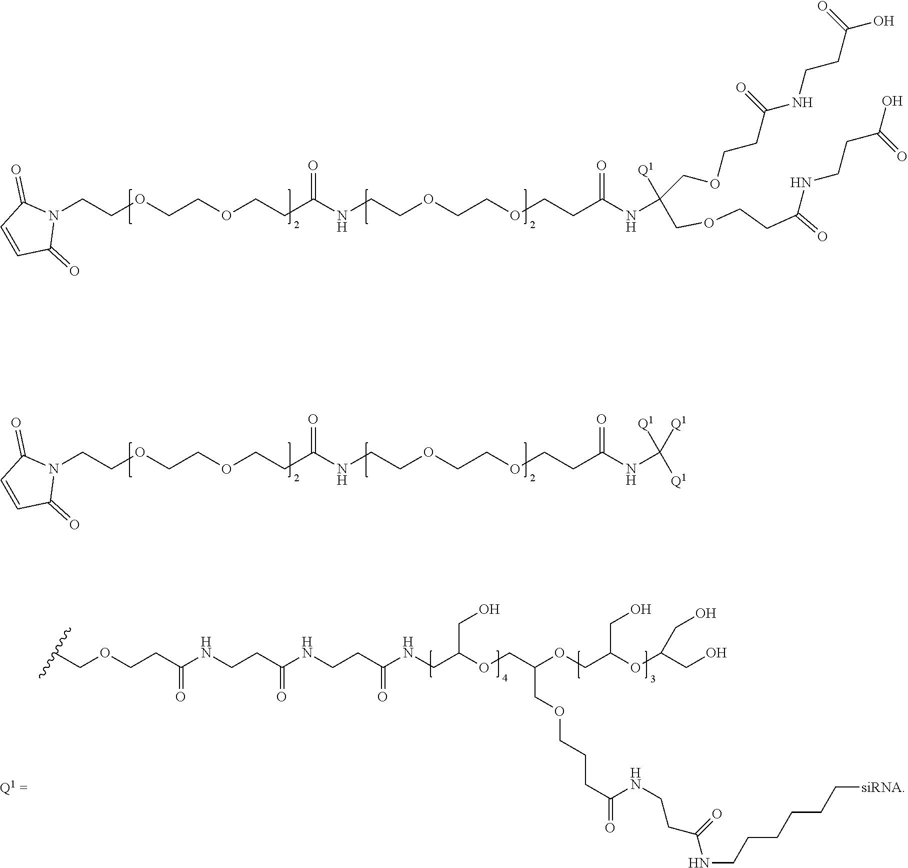

The synthesis scheme of antibody siRNA conjugates provided herein is shown below:

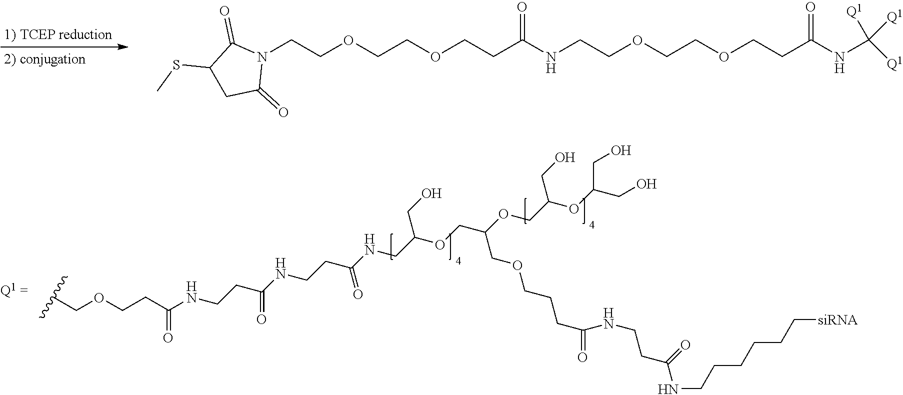

siRNA 23 was selected and coupled with αvβ6 antibody as described below:

To the solution of the h2A2 antibody (of which the heavy chain is shown as SEQ ID NO: 264, and the light chain is shown as SEQ ID NO: 265, see WO2021113697) in conjugation buffer (PBS buffer), 5 eq. of TCEP was added. The mixture was shaken for 1 hour at room temperature. To the reduced antibody solution, 10 eq. of Linker-Polymer-Oligonucleotide compound was added. The reaction mixture was mixed well and shaken for 1 hour. Then 50 eq. of L-cysteine solution was added to reaction mixture to quench the reaction. The reaction mixture was shaken for another 30 minutes at room temperature. Then, the conjugate was purified via cation exchange column (3 mg, 2 mg/ml). DAR was determined by UV spectrometer.

TABLE 6
h2A2 antibody sequence.

| SEQ | |
|---|---|
| ID NO. | SEQUENCE |
| 264 | QFQLVQSGAEVKKPGASVKVSCKASGYSFTDYNVNWVRQAP |
| | GQGLEWIGVINPKYGTTRYNQKFKGRATLTVDKSTSTAYME |
| | LSSLRSEDTAVYYCTRGLNAWDYWGQGTLVTVSSASTKGPS |
| | VFPLAPSSKSTSGGTAALGCLVKDYFPEPVTVSWNSGALTS |
| | GVHTFPAVLQSSGLYSLSSVVTVPSSSLGTQTYICNVNHKP |
| | SNTKVDKKVEPKSCDKTHTCPPCPAPELLGGPSVFLFPPKP |
| | KDTLMISRTPEVTCVVVDVSHEDPEVKFNWYVDGVEVHNAK |
| | TKPREEQYNSTYRVVSVLTVLHQDWLNGKEYKCKVSNKALP |
| | APIEKTISKAKGQPREPQVYTLPPSREEMTKNQVSLTCLVK |
| | GFYPSDIAVEWESNGQPENNYKTTPPVLDSDGSFFLYSKLT |
| | VDKSRWQQGNVFSCSVMHEALHNHYTQKSLSLSPGK |
| 265 | DIQMTQSPSSLSASVGDRVTITCGASENIYGALNWYQQKPG |
| | KAPKLLIYGATNLEDGVPSRFSGSGSGRDYTFTISSLQPED |
| | IATYYCQNVLTTPYTFGQGTKLEIKRTVAAPSVFIFPPSDE |
| | QLKSGTASVVCLLNNFYPREAKVQWKVDNALQSGNSQESVT |
| | EQDSKDSTYSLSSTLTLSKADYEKHKVYACEVTHQGLSSPV |
| | TKSFNRGEC |

The silencing efficiency of the antibody siRNA conjugate was tested with PANC-1 cell using the protocol as described in example 6, at the concentration of 50 nM. The silencing efficacy was 35.9%.

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

The words “comprise”, “comprising”, “include”, “including”, and “includes” when used in this specification and in the following claims are intended to specify the presence of stated features, integers, components, or steps, but they do not preclude the presence or addition of one or more other features, integers, components, steps, or groups thereof.

What claimed is:

1. A polymeric scaffold of Formula (I) or Formula (II) useful to conjugate with a targeting moiety: 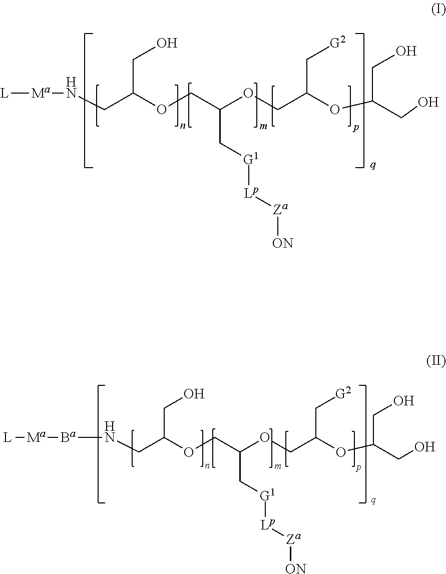
wherein:
the polymeric scaffold comprises linear polyglycerol;
L is a linking moiety comprising a functional group Wₚ that is capable of forming a covalent bond with the targeting moiety;
M^{a} is a stretcher connecting L to B^{a} or —NH— moiety;
B^{a} is a branching moiety comprising a functional group W_{M} connecting to —NH-moiety;
each G¹ is independently a functional group connecting L^{p} to the linear polyglycerol;
each L^{p} is independently a drug release mechanism between G¹ and Z^{a};
each Z^{a} is independently an endosomal-lysosomal escaping moiety or a conjugation moiety;
each ON is independently an oligonucleotide;
each G² is independently a functional group capable of converting into a charged state;
n is an integer from 0 to 1000;
m is an integer from 0 to 1000;
p is an integer from 0 to 1000; and
q is an integer from 0 to 1000.

2. The polymeric scaffold of claim 1, wherein Wₚ is capable of reacting with a functional group on the targeting moiety with a click reaction.

3. The polymeric scaffold of claim 2, wherein Wₚ is selected from the group consisting of:

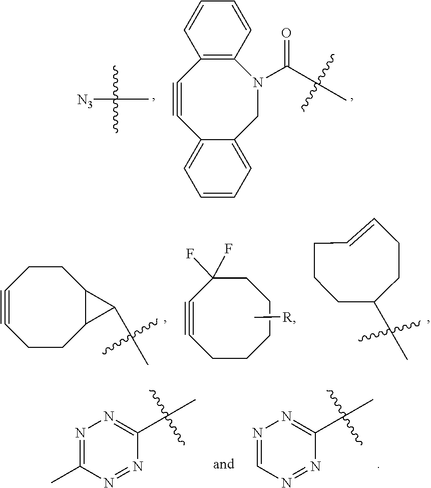

4. The polymeric scaffold of claim 1, wherein Wₚ is capable of reacting with amino acids on the targeting moiety.

5. The polymeric scaffold of claim 4, wherein the amino acids are natural amino acids, non-natural amino acids or combination thereof.

6. The polymeric scaffold of claim 5, wherein the natural amino acid comprises cysteine, lysine, tyrosine, aspartic acid and glutamic acid.

7. The polymeric scaffold of claim 4, wherein Wₚ is capable of reacting with one or more cysteines on the targeting moiety.

8. The polymeric scaffold of claim 7, wherein Wₚ is selected from the group consisting of: 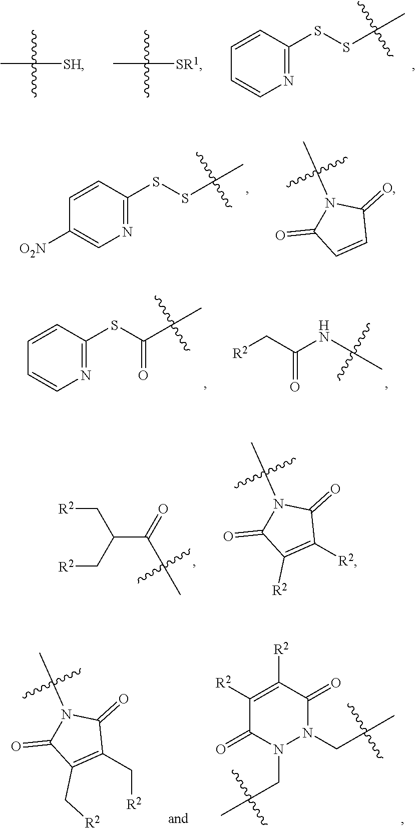
wherein R¹ is a sulfur protecting group, each R² is independently a leaving group.

9. The polymeric scaffold of claim 8, wherein each R² is independently selected from halo or R^{2a}C(O)O—, in which R^{2a} is hydrogen, an aliphatic, heteroaliphatic, cycloalkyl, heterocycloalkyl, aryl or heteroaryl.

10. The polymeric scaffold of claim 4, wherein Wₚ is capable of reacting with one or more lysines on the targeting moiety, in which each Wₚ is independently selected from the group consisting of:

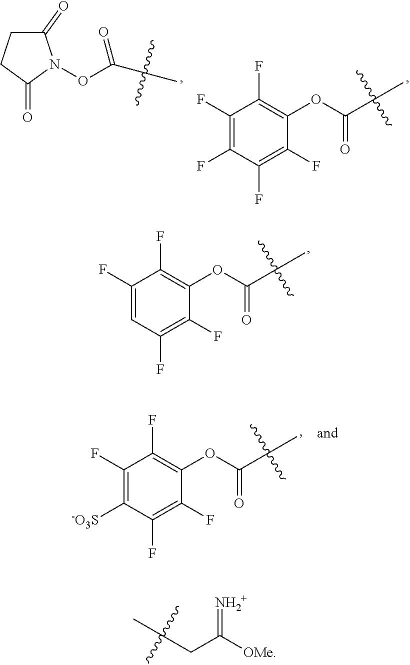

11. The polymeric scaffold of claim 4, wherein Wₚ is capable of reacting with one or more non-natural amino acids on the targeting moiety.

12. The polymeric scaffold of claim 11, wherein Wₚ is selected from:

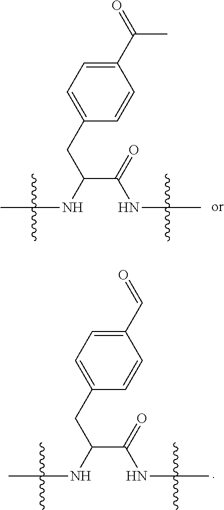 [SMILES: *NC(=O)C(Cc1ccc(C(C)=O)cc1)N* ; *NC(=O)C(Cc1ccc(C=O)cc1)N*]

13. The polymeric scaffold of any one of preceding claims, wherein L is

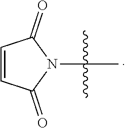 [SMILES: *N1C(=O)C=CC1=O]

14. The polymeric scaffold of claim 1, wherein M^{a} is selected from the group consisting of: 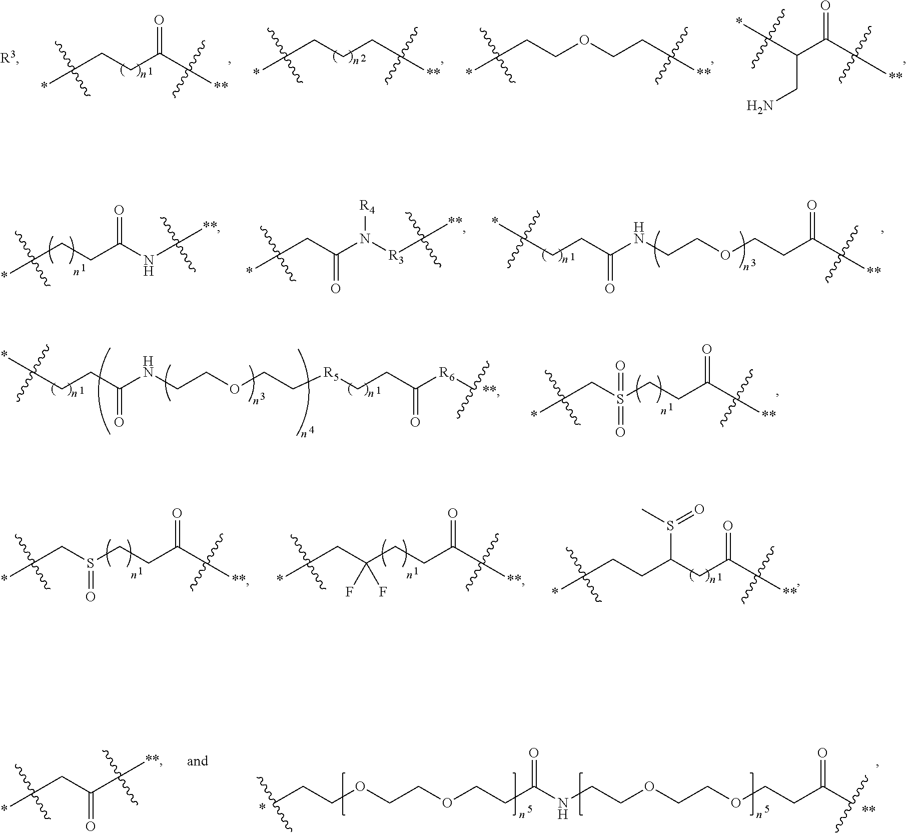
wherein * is the site covalently attached to L, ** is the site covalently attached to B^{a} or —NH— moiety,
R³ is C₁₋₁₀ alkyl, C₁₋₁₀ heteroalkyl, C₃₋₈ cycloalkyl, —O—(C₁₋₈ alkyl)-, aryl, —C₁₋₁₀ alkyl-aryl-, -aryl-C₁₋₁₀ alkyl-, —C₁₋₁₀ alkyl-(C₃₋₈ cycloalkyl)-, —(C₃₋₈ cycloalkyl-C₁₋₁₀ alkyl)-, 4- to 14-membered heterocycloalkyl, —C₁₋₁₀ alkyl-(4- to 14-membered heterocycloalkyl)-, -(4- to 14-membered heterocycloalkyl)-C₁₋₁₀ alkyl-, —C₁₋₁₀ alkyl-C(═O)—, —C₁₋₁₀ heteroalkyl-C(═O)—, —C₃₋₈ cycloalkyl-C(═O)—, —O—(C₁₋₈ alkyl)-C(═O)—, -aryl-C(═O)—, —C₁₋₁₀ alkyl-aryl-C(═O)—, -aryl-C₁₋₁₀ alkyl-C(═O)—, —C₁₋₁₀ alkyl-(C₃₋₈ cycloalkyl)-C(═O)—, —(C₃₋₈ cycloalkyl)-C₁₋₁₀ alkyl-C(═O)—, -4- to 14-membered heterocycloalkyl-C═(O)—, —C₁₋₁₀ alkyl-(4- to 14-membered heterocycloalkyl)-C(═O)—, -(4- to 14-membered heterocycloalkyl)-C₁₋₁₀ alkyl-C(═O)—, —C₁₋₁₀ alkyl-NH—, —C₁₋₁₀ heteroalkyl-NH—, —C₃₋₈ cycloalkyl-NH—, —O—(C₁₋₈ alkyl)-NH—, -aryl-NH—, —C₁₋₁₀ alkyl-aryl-NH—, -aryl-C₁₋₁₀ alkyl-NH—, —C₁₋₁₀ alkyl-(C₃₋₈ cycloalkyl)-NH—, —(C₃₋₈ cycloalkyl)-C₁₋₁₀ alkyl-NH—, -4- to 14-membered heterocycloalkyl-NH—, —C₁₋₁₀ alkyl-(4- to 14-membered heterocycloalkyl)-NH—, -(4- to 14-membered heterocycloalkyl)-C₁₋₁₀ alkyl-NH—, —C₁₋₁₀ alkyl-S—, —C₁₋₁₀ heteroalkyl-S—, —C₃₋₈ cycloalkyl-S—, —O—C₁₋₈ alkyl-S—, -aryl-S—, —C₁₋₁₀ alkyl-aryl-S—, -aryl-C₁₋₁₀ alkyl-S—, —C₁₋₁₀ alkyl-(C₃₋₈ cycloalkyl)-S—, —(C₃₋₈ cycloalkyl)-C₁₋₁₀ alkyl-S—, -4- to 14-membered heterocycloalkyl-S—, —C₁₋₁₀ alkyl-(4- to 14-membered heterocycloalkyl)-S—, or -(4- to 14-membered heterocycloalkyl)-C₁₋₁₀ alkyl-S—;
each R⁴ independently is hydrogen, C₁₋₆ alkyl, C₆₋₁₀ aryl, C₃₋₈ cycloalkyl, —COOH or —COO—C₁₋₆ alkyl;
R⁵ is —C(O)—NR^{5a} or —NR^{5a}—C(O)—;
R^{5a} is hydrogen, C₁₋₆ alkyl, C₆₋₁₀ aryl, C₃₋₈ cycloalkyl, —COOH or —COO—C₁₋₆ alkyl;
R⁶ is a bond or —NR^{6a}—(CR^{6b}R^{6c})—C(O)—;
R^{6a} is hydrogen, C₁₋₆ alkyl, C₆₋₁₀ aryl, C₃₋₈ cycloalkyl, —COOH or —COO—C₁₋₆ alkyl;
each R^{6b} and R^{6c} independently is hydrogen, C₁₋₆ alkyl, C₆₋₁₀ aryl, hydroxylated C₆₋₁₀ aryl, polyhydroxylated C₆₋₁₀ aryl, 5- to 12-membered heterocycloalkyl, C₃₋₈ cycloalkyl, hydroxylated C₃₋₈ cycloalkyl, polyhydroxylated C₃₋₈ cycloalkyl or a side chain of a natural or unnatural amino acid;
each n¹ independently is an integer from 0 to 6;
n² is an integer from 0 to 8;
each n³ independently is an integer from 1 to 6;
n⁴ is an integer from 1 to 4; and
n⁵ is an integer from 1 to 4.

15. The polymeric scaffold of claim 13, wherein M^{a} is selected from the group consisting of:

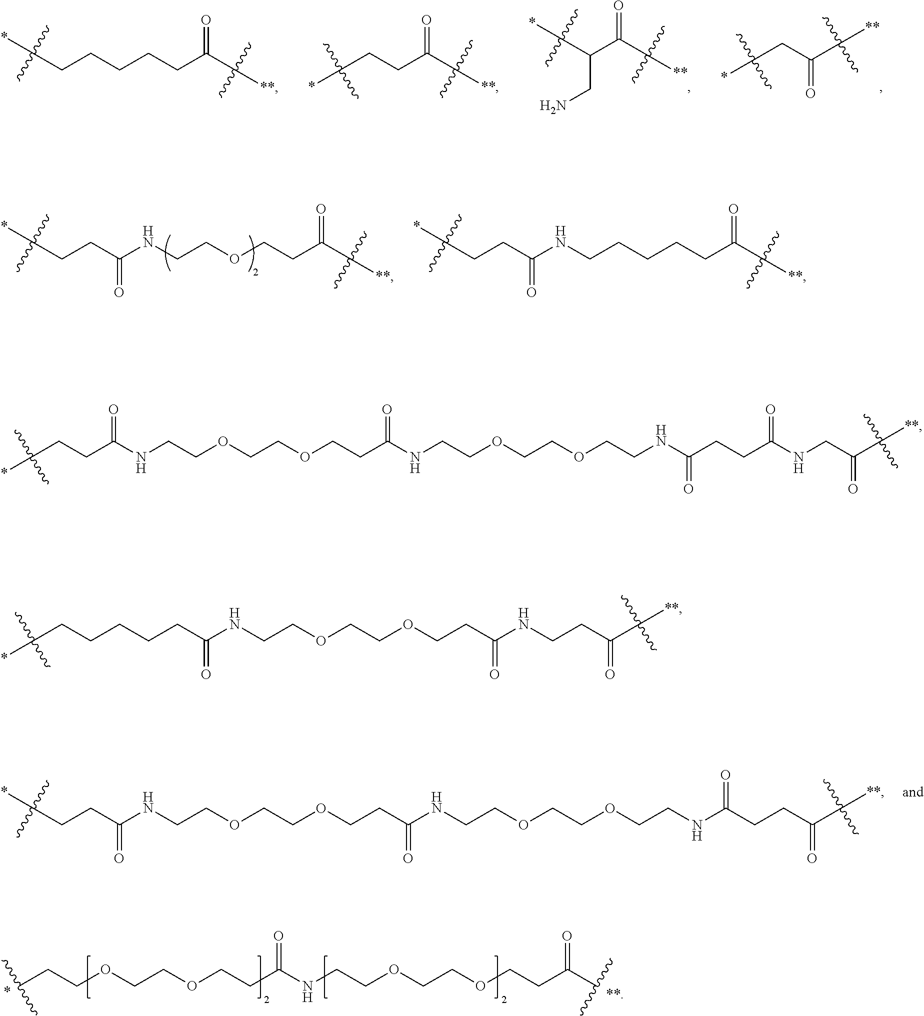

16. The polymeric scaffold of claim 1, wherein G¹ is selected from the group consisting of: 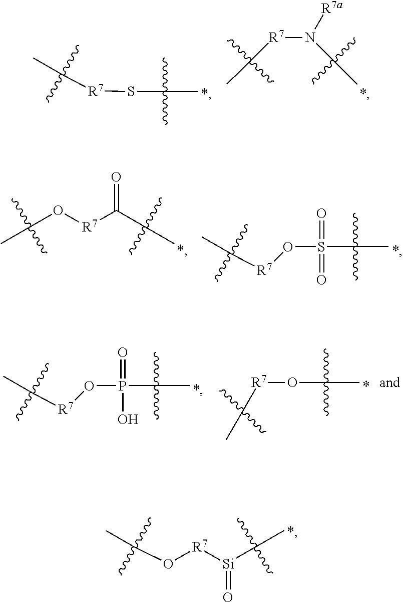
wherein * is the site covalently attached to L^{P}, each R⁷ is independently selected from a direct bond, alkyl, cycloalkyl, heterocycloalkyl, aryl, or heteroaryl, and R^{7a} is selected from hydrogen, an aliphatic, heteroaliphatic, cycloalkyl, or heterocycloalkyl moiety.

17. The polymeric scaffold of claim 1, wherein each L^{p} independently comprises a labile structure.

18. The polymeric scaffold of claim 17, wherein the labile structure is selected from redox labile structures, hydrolytically labile structures or enzymatic labile structures.

19. The polymeric scaffold of claim 18, wherein the labile structure is a redox labile structure having a structure of 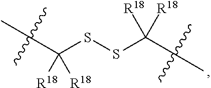
wherein each of R¹⁸ is independently selected from hydrogen, aliphatic, heteroaliphatic, cycloalkyl or heterocycloalkyl.

20. The polymeric scaffold of claim 18, wherein the hydrolytically labile structure is selected from the group consisting of: 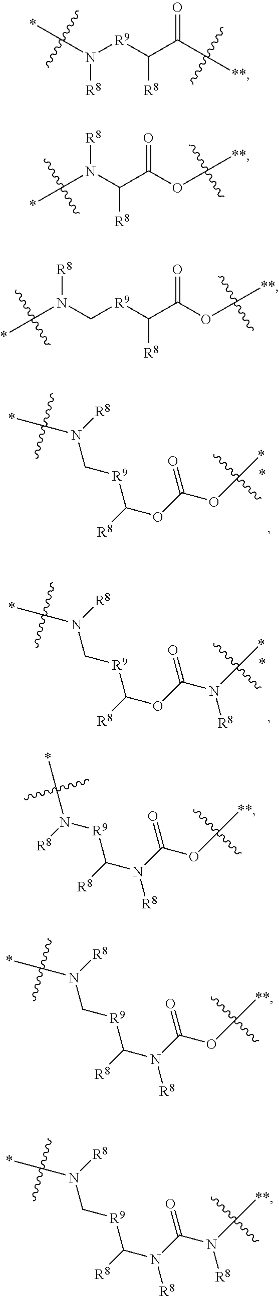 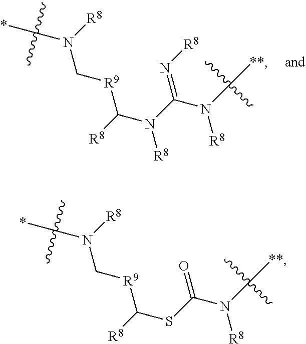
wherein * is the site covalently attached to G¹, ** is the site covalently attached to Z^{a}, R⁸ is selected from hydrogen, alkyl or aryl, R⁹ is selected from aliphatic, heteroaliphatic, cycloalkyl, heterocycloalkyl, aryl, or heteroaryl.

21. The polymeric scaffold of claim 20, wherein G¹ is 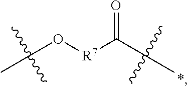
wherein R⁷ is alkyl.

22. The polymeric scaffold of claim 18, wherein the labile structure is an enzymatic labile structure is liable to enzymes selected from Cathepsin B, phosphatase, sulfatase, or glucuronidase.

23. The polymeric scaffold of claim 22, wherein the enzymatic labile structure is liable to cathepsin B and is selected from —Z— or 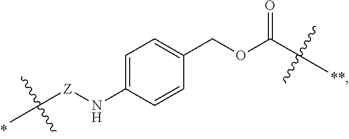
wherein Z is a substrate for cathepsin B comprising 2 to 4 amino acids.

24. The polymeric scaffold of claim 23, wherein G¹ is 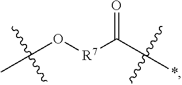
wherein R⁷ is alkyl.

25. The polymeric scaffold of claim 22, wherein the enzymatic labile structure is liable to glucuronidase and is 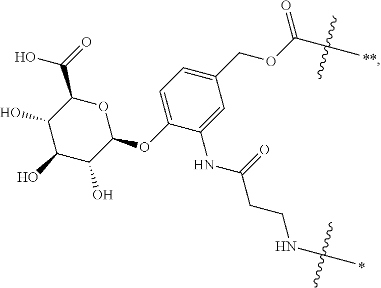 [SMILES: *NCCC(=O)Nc1cc(COC(*)=O)ccc1O[C@@H]1O[C@H](C(=O)O)[C@@H](O)[C@H](O)[C@H]1O]
wherein * is the site covalently attached to G¹, ** is the site covalently attached to Z^{a}.

26. The polymeric scaffold of claim 25, wherein G¹ is 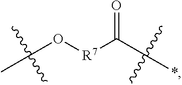
wherein R⁷ is alkyl.

27. The polymeric scaffold of claim 22, wherein the enzymatic labile structure is liable to phosphatase and is selected from 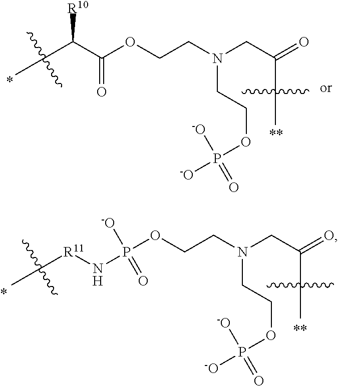
wherein each of R¹⁰ and R¹¹ is independently hydrogen, an aliphatic, heteroaliphatic, cycloalkyl, heterocycloalkyl, aryl or heteroaryl.

28. The polymeric scaffold of claim 27, wherein G¹ is 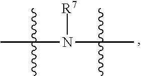
wherein R⁷ is selected from hydrogen, an aliphatic, heteroaliphatic, cycloalkyl, heterocycloalkyl, aryl or heteroaryl.

29. The polymeric scaffold of claim 22, wherein the enzymatic labile structures are liable to sulfatase and is 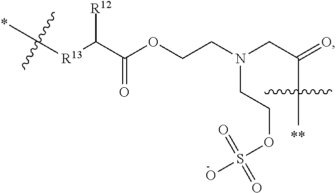
wherein each of R¹² and R¹³ is independently hydrogen, an aliphatic, heteroaliphatic, cycloalkyl, heterocycloalkyl, aryl or heteroaryl.

30. The polymeric scaffold of claim 29, wherein G¹ is 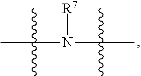
wherein R⁷ is selected from hydrogen, an aliphatic, heteroaliphatic, cycloalkyl, heterocycloalkyl, aryl or heteroaryl.

31. The polymeric scaffold of claim 1, wherein Z^{a} is a cell penetration peptide.

32. The polymeric scaffold of claim 31, wherein Z^{a} is selected from the group consisting of Tat, Penetratin, Pep-1, MPG, Polyarginine (R9, R8), R6/W3, SAP, SAP(E), CyLop-1, gH 625, GALA, TP10, CADY, L17E, MPPs, Ac-1, Ent Ac-1, Ac-2, Peptide 3, RR5-App, RR4-App, RR3-aPP, TATp-D, R4-R4, R5-R5, [WR]₄, Cyclic Tat, cFΦR₄, Danamide D, Pro-(Xaa)₄-Tyr, Cyclic sC18, RRRRΦF, BIM SAHB9, SAH-SOS1, 4-R, 4-W, and Sp-CC-PEG₂₀₀₀.

33. The polymeric scaffold of claim 1, wherein Z^{a} is an endosomal escape domain.

34. The polymeric scaffold of claim 33, wherein Z^{a} is selected from the group consisting of GG, GFFG, GFWFG, GWGGWG, GWG, GFWG, GWWG, and GWWG.

35. The polymeric scaffold of claim 1, wherein Z^{a} is a cationic polymer.

36. The polymeric scaffold of claim 35, wherein Z^{a} is selected from PEI, PLL, PLA, DEAE-DEX or PAMAM.

37. The polymeric scaffold of claim 1, wherein Z^{a} is a conjugation moiety.

38. The polymeric scaffold of claim 37, wherein Z^{a} is **—NH—(CH₂)ₙ₇—* or **—S—(CH₂)ₙ₇—*, wherein n7 is an integer from 1 to 6, * is the site covalently attached to ON, and ** is the site covalently attached to L^{p}.

39. The polymeric scaffold of claim 38, wherein Z^{a} is —NH—(CH₂)₆—.

40. The polymeric scaffold of claim 1, wherein -G¹-L^{p}-Z^{a}— is 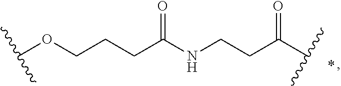 [SMILES: *OCCCC(=O)NCCC(*)=O]
wherein * is the site covalently attached to ON.

41. The polymeric scaffold of claim 1, wherein
n is an integer from 1 to 100;
m is an integer from 1 to 100; and
p is an integer from 1 to 50.

42. The polymeric scaffold of claim 1, wherein ON is selected from the group consisting of antisense oligonucleotides, mRNA, siRNA, saRNA, miRNA, shRNA and CRISPER/Cas9 complex.

43. The polymeric scaffold of claim 42, wherein ON silences the TGFβ.

44. The polymeric scaffold of claim 42, wherein ON is a double strand siRNA.

45. The polymeric scaffold of claim 44, wherein sense strand sequence of the siRNA is shown as SEQ ID NO.: 203, and antisense strand sequence of the siRNA is shown as SEQ ID NO.: 204.

46. The polymeric scaffold of claim 45, wherein Z^{a} is connected to ON at 5′ or 3′ end.

47. The polymeric scaffold of claim 1, wherein G² is selected from the group consisting of: 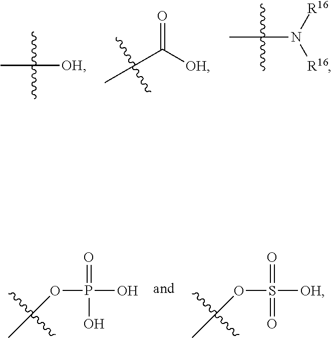
wherein each R¹⁶ is independently hydrogen, an aliphatic, heteroaliphatic, cycloalkyl, heterocycloalkyl, aryl or heteroaryl.

48. The polymeric scaffold of claim 1, wherein B^{a} is 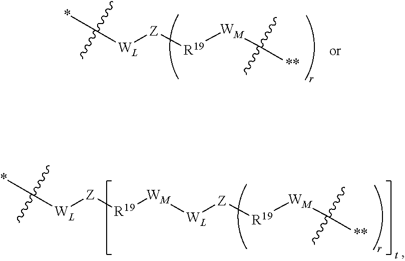
wherein W_{L} is a functional group connected to stretcher M^{a};
each Z is independently a branching point;
each R¹⁹ is independently a linker connecting Z to W_{M};
each W_{M} is independently a functional group connected to —NH—;
r is an integer from 1-3;
t is an integer from 1-3;
wherein * is the site covalently attached to M^{a}, ** is the site covalently attached to —NH-moiety.

49. The polymeric scaffold of claim 48, wherein the —NH— moiety is 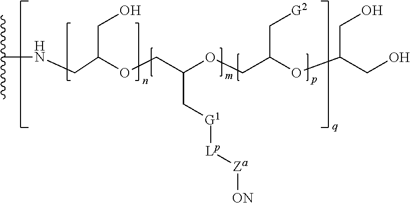
or —NH—(CH₂)ₙ₆—COOH, wherein G¹, L^{p}, Z^{a}, G², n, m, p and q are as defined in claim 1, and n⁶ is an integer from 1 to 6.

50. The polymeric scaffold of claim 48, wherein W_{L} is selected from a group consisting of:

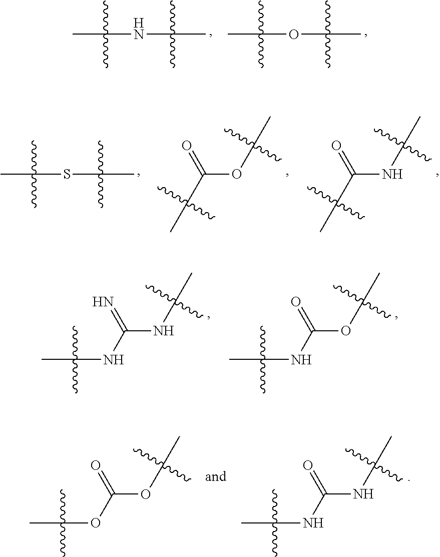

51. The polymeric scaffold of claim 48, wherein Z is —CH₍₃₋ᵣ₎-, -SiH₍₃₋ᵣ₎— or -NH₍₂₋ᵣ₎—.

52. The polymeric scaffold of claim 48, wherein R¹⁹ is selected from a group consisting of an aliphatic, heteroaliphatic, cycloalkyl, or heterocycloalkyl.

53. The polymeric scaffold of claim 48, wherein B^{a} is

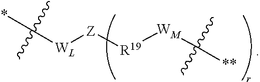

54. The polymeric scaffold of claim 53, wherein B^{a} is selected from the group consisting of

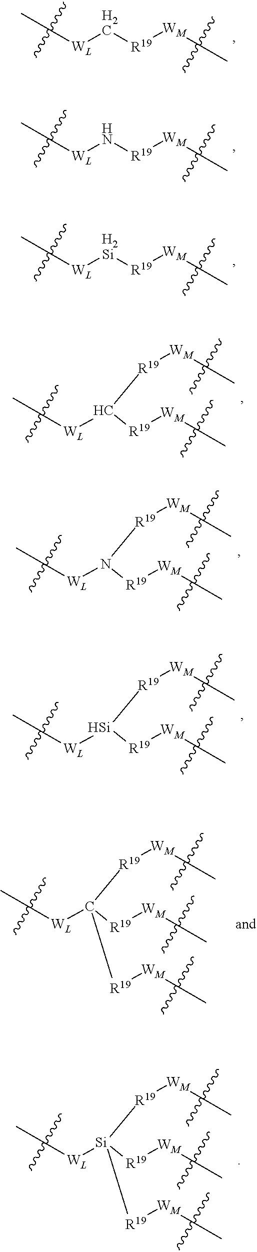

55. The polymeric scaffold of claim 48, wherein B^{a} is

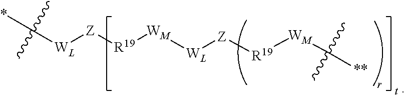

56. The polymeric scaffold of claim 55, wherein B^{a} is selected from the group consisting of:

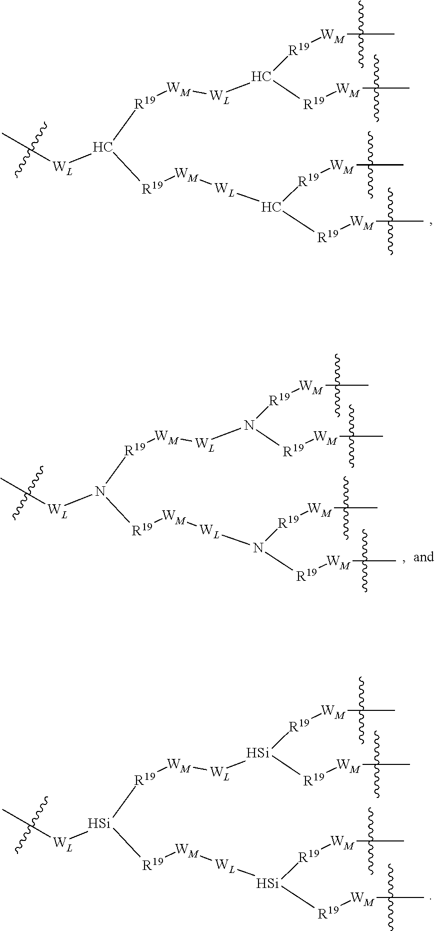

57. The polymeric scaffold of any one of claims 1-56, wherein B^{a} is

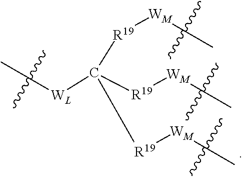

58. The polymeric scaffold of claim 57, wherein two of the W_{M} are connected to —NH—(CH₂)ₙ₆—COOH, and one of the W_{M} is connected to

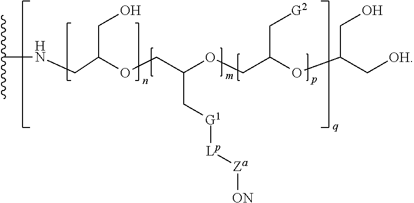

59. The polymeric scaffold of claim 57, wherein three W_{M} are connected to

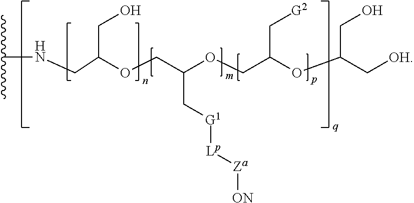

60. The polymeric scaffold of any one of claims 53-59, wherein W_{L} is 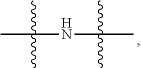 [SMILES: *N*]
and —R⁹—W_{M}— is 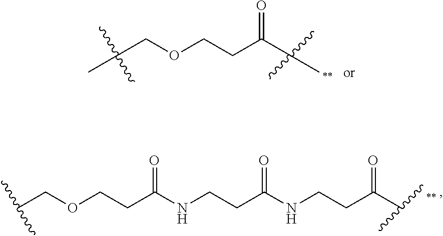 [SMILES: *COCCC(*)=O ; *COCCC(=O)NCCC(=O)NCCC(*)=O]
wherein ** is the site covalently attached to W_{L} or —NH— moiety.

61. The polymeric scaffold of claim 55, wherein B^{a} is

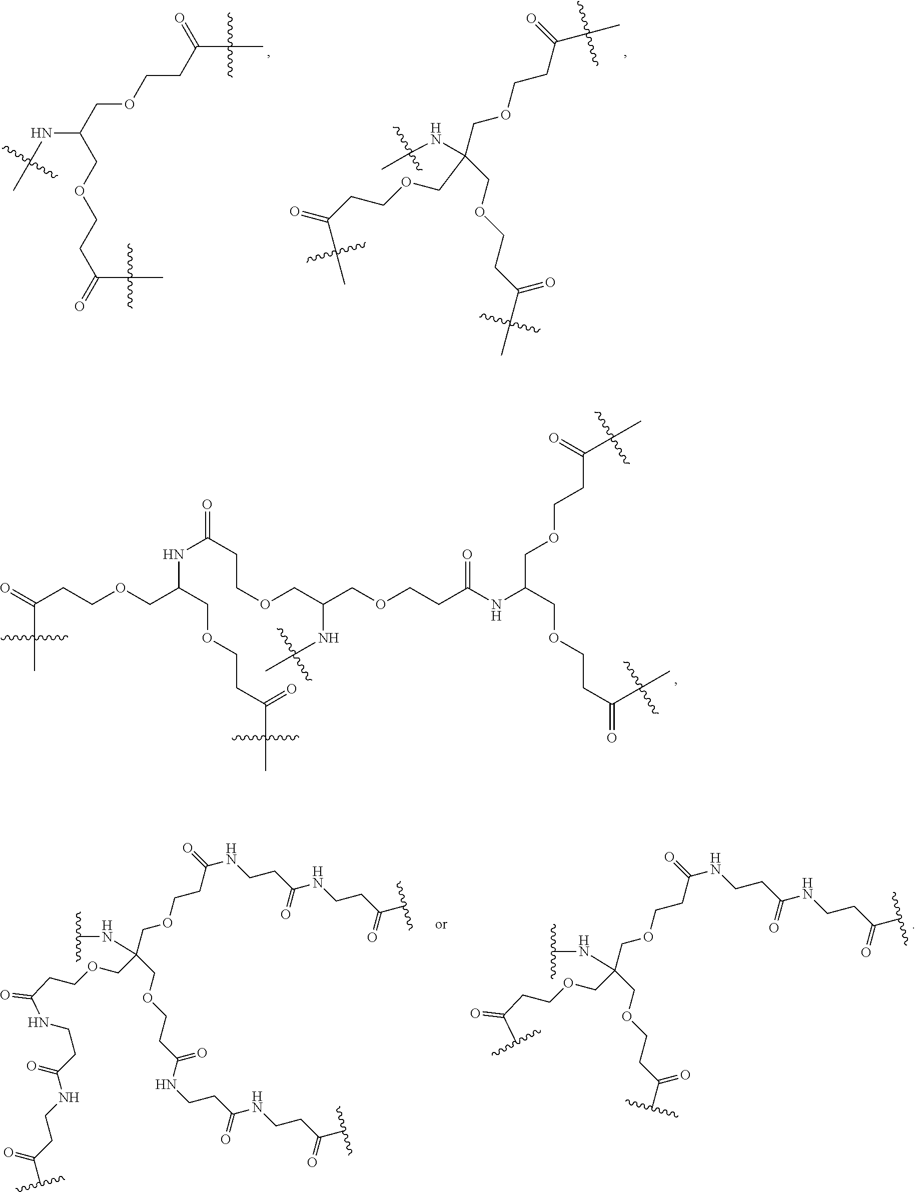 [SMILES: *NC(COCCC(*)=O)COCCC(*)=O ; *NC(COCCC(*)=O)(COCCC(*)=O)COCCC(*)=O ; *NC(COCCC(=O)NC(COCCC(*)=O)COCCC(*)=O)COCCC(=O)NC(COCCC(*)=O)COCCC(*)=O ; *NC(COCCC(=O)NCCC(=O)NCCC(*)=O)(COCCC(=O)NCCC(=O)NCCC(*)=O)COCCC(=O)NCCC(=O)NCCC(*)=O ; *NC(COCCC(*)=O)(COCCC(*)=O)COCCC(=O)NCCC(=O)NCCC(*)=O]

62. The polymeric scaffold of claim 1, selected from the group consisting of:

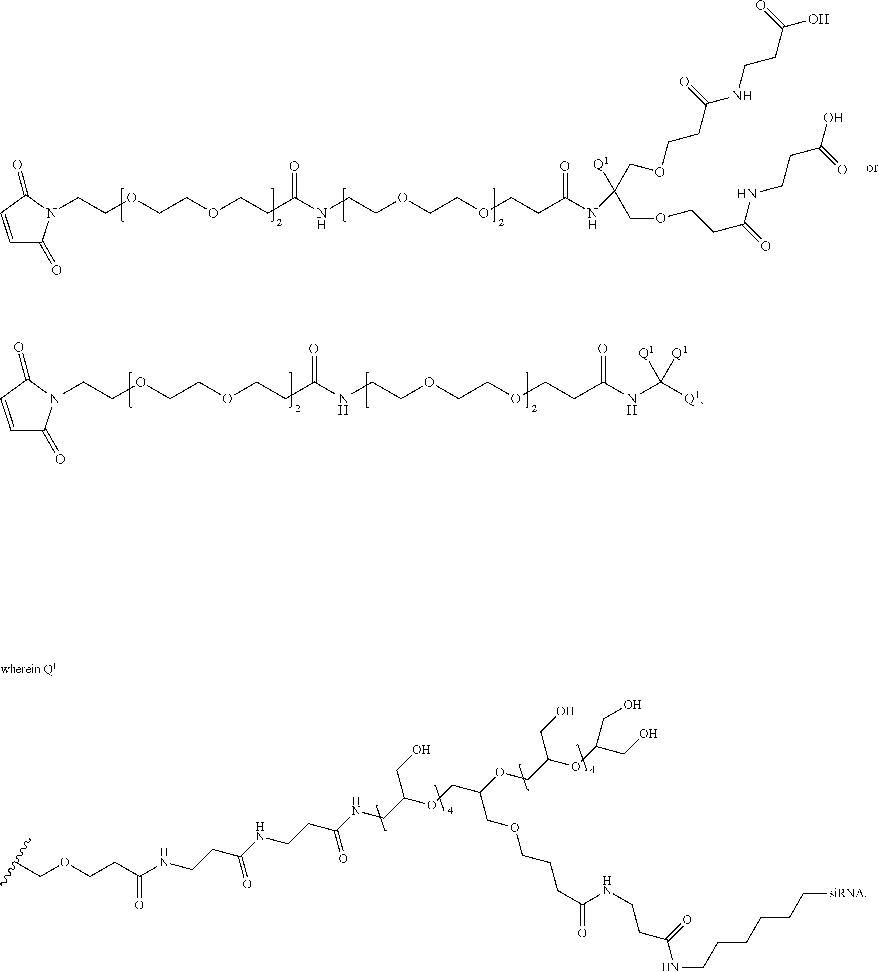

63. A polymeric scaffold of Formula (III) or Formula (IV): 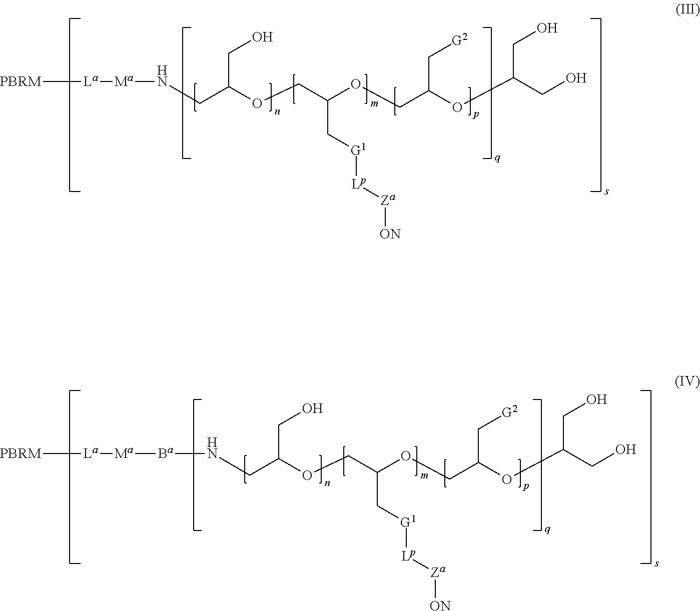
wherein,
the polymeric scaffold comprises linear polyglycerol;
PBRM is a targeting moiety;
each L^{a} is independently a divalent moiety connecting the targeting moiety to M^{a};
each M^{a} is independently a stretcher connecting L^{a} to B^{a} or —NH— moiety;
B^{a} is branching moiety comprising a functional group W_{M} connecting to —NH— moiety;
each G′ is independently a functional group connecting L^{P} to the linear polyglycerol;
each L^{P} is independently a drug release mechanism between G′ and Z^{a};
each Z^{a} is independently a lysosomal escaping moiety or a conjugation moiety;
each ON is independently an oligonucleotide;
each G² is independently a functional group capable of converting into a charged state;
n is an integer from 0 to 1000;
m is an integer from 0 to 1000;
p is an integer from 0 to 1000;
q is an integer from 0 to 1000; and
s is an integer from 1 to 8.

64. The polymeric scaffold of claim 63, wherein the targeting moiety is an antibody and/or fragment thereof.

65. The polymeric scaffold of claim 63, wherein the targeting moiety is an antibody IgG1, IgG2, IgG3, and IgG4.

66. The polymeric scaffold of claim 63, wherein the targeting moiety is selected from the group consisting of a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer, a bispecific antibody, a trispecific antibody, a bi-paratropic antibody, a multispecific antibody, a camelized single domain antibody, a nanobody, a domain antibody, a bivalent domain antibody, or VHH.

67. The polymeric scaffold of claim 63, wherein the targeting moiety is anti-αvβ6 antibody, or antigen-binding fragment thereof.

68. The polymeric scaffold of claim 63, wherein the polymeric scaffold is

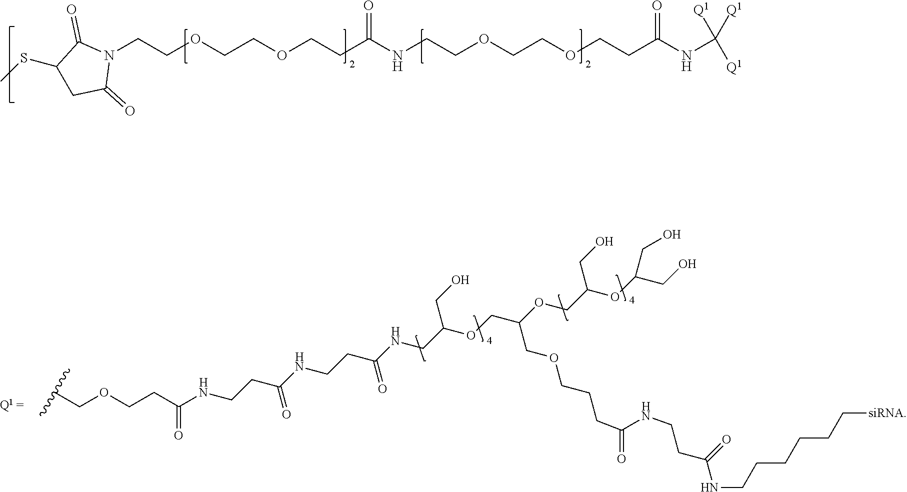

69. A pharmaceutical composition comprising one or more of the polymeric scaffold according to any one of claims 1-68 and an acceptable carrier.

70. A method of treating a disorder in a subject in need thereof, the method comprising administering to the subject a therapeutic effective amount of the polymeric scaffold according to any one of claims 1-68 or the pharmaceutical composition according to claim 69.


SEQUENCE LISTING (265 sequences)

SEQ ID NO: 1 - RNA, 21 nt, synthetic construct
  source 1..21: mol_type = other RNA
cacactgcaagtggacatcaa

SEQ ID NO: 2 - RNA, 21 nt, synthetic construct
  source 1..21: mol_type = other RNA
cacatcagagctccgagaagc

SEQ ID NO: 3 - RNA, 21 nt, synthetic construct
  source 1..21: mol_type = other RNA
cactactgtagttagatctat

SEQ ID NO: 4 - RNA, 21 nt, synthetic construct
  source 1..21: mol_type = other RNA
gcctgtctgcactattccttt

SEQ ID NO: 5 - RNA, 21 nt, synthetic construct
  source 1..21: mol_type = other RNA
gagacggatctctctccgacc

SEQ ID NO: 6 - RNA, 21 nt, synthetic construct
  source 1..21: mol_type = other RNA
ggccctgtacaaccagcataa

SEQ ID NO: 7 - RNA, 21 nt, synthetic construct
  source 1..21: mol_type = other RNA
gaacactactgtagttagatc

SEQ ID NO: 8 - RNA, 21 nt, synthetic construct
  source 1..21: mol_type = other RNA
gcccatctaggttatttccgt

SEQ ID NO: 9 - RNA, 21 nt, synthetic construct
  source 1..21: mol_type = other RNA
ggacaccaactattgcttcag

SEQ ID NO: 10 - RNA, 21 nt, synthetic construct
  source 1..21: mol_type = other RNA
gcggtacctgaacccgtgttg

SEQ ID NO: 11 - RNA, 21 nt, synthetic construct
  source 1..21: mol_type = other RNA
gcccctacatttggagcctgg

SEQ ID NO: 12 - RNA, 21 nt, synthetic construct
  source 1..21: mol_type = other RNA
gcaagactatcgacatggagc

SEQ ID NO: 13 - RNA, 21 nt, synthetic construct
  source 1..21: mol_type = other RNA
gactcgccagagtggttatct

SEQ ID NO: 14 - RNA, 21 nt, synthetic construct
  source 1..21: mol_type = other RNA
gccacagatcccctattcaag

SEQ ID NO: 15 - RNA, 21 nt, synthetic construct
  source 1..21: mol_type = other RNA
ggtggaaacccacaacgaaat

SEQ ID NO: 16 - RNA, 21 nt, synthetic construct
  source 1..21: mol_type = other RNA
gccgtggaggggaaattgagg

SEQ ID NO: 17 - RNA, 21 nt, synthetic construct
  source 1..21: mol_type = other RNA
gccccattaaagatggagaga

SEQ ID NO: 18 - RNA, 21 nt, synthetic construct
  source 1..21: mol_type = other RNA
gtggggaacactactgtagtt

SEQ ID NO: 19 - RNA, 21 nt, synthetic construct
  source 1..21: mol_type = other RNA
gcagctgtccaacatgatcgt

SEQ ID NO: 20 - RNA, 21 nt, synthetic construct
  source 1..21: mol_type = other RNA
gccctgtacaaccagcataac

SEQ ID NO: 21 - RNA, 21 nt, synthetic construct
  source 1..21: mol_type = other RNA
gagaggactgcggatctctgt

SEQ ID NO: 22 - RNA, 21 nt, synthetic construct
  source 1..21: mol_type = other RNA
gggaacactactgtagttaga

SEQ ID NO: 23 - RNA, 21 nt, synthetic construct
  source 1..21: mol_type = other RNA
gtggaaacccacaacgaaatc

SEQ ID NO: 24 - RNA, 21 nt, synthetic construct
  source 1..21: mol_type = other RNA
gcaacactctgagatgcaggg

SEQ ID NO: 25 - RNA, 21 nt, synthetic construct
  source 1..21: mol_type = other RNA
cggcagctgtacattgacttc

SEQ ID NO: 26 - RNA, 21 nt, synthetic construct
  source 1..21: mol_type = other RNA
gacaccaactattgcttcagc

SEQ ID NO: 27 - RNA, 21 nt, synthetic construct
  source 1..21: mol_type = other RNA
gcagtacagcaaggtcctggc

SEQ ID NO: 28 - RNA, 21 nt, synthetic construct
  source 1..21: mol_type = other RNA
gctgtccaacatgatcgtgcg

SEQ ID NO: 29 - RNA, 21 nt, synthetic construct
  source 1..21: mol_type = other RNA
ccatagcaacactctgagatg

SEQ ID NO: 30 - RNA, 21 nt, synthetic construct
  source 1..21: mol_type = other RNA
gttgtgcggcagtggttgagc

SEQ ID NO: 31 - RNA, 21 nt, synthetic construct
  source 1..21: mol_type = other RNA
gacctggccaccattcatggc

SEQ ID NO: 32 - RNA, 21 nt, synthetic construct
  source 1..21: mol_type = other RNA
gcagagtacacacagcatata

SEQ ID NO: 33 - RNA, 21 nt, synthetic construct
  source 1..21: mol_type = other RNA
gcggcagctgtacattgactt

SEQ ID NO: 34 - RNA, 21 nt, synthetic construct
  source 1..21: mol_type = other RNA
gtctgcactattcctttgccc

SEQ ID NO: 35 - RNA, 21 nt, synthetic construct
  source 1..21: mol_type = other RNA
gaggttaagggaggagttcct

SEQ ID NO: 36 - RNA, 21 nt, synthetic construct
  source 1..21: mol_type = other RNA
gcatcctagaccctttctcct

SEQ ID NO: 37 - RNA, 21 nt, synthetic construct
  source 1..21: mol_type = other RNA
gcaacaattcctggcgatacc

SEQ ID NO: 38 - RNA, 21 nt, synthetic construct
  source 1..21: mol_type = other RNA
gcagggataacacactgcaag

SEQ ID NO: 39 - RNA, 21 nt, synthetic construct
  source 1..21: mol_type = other RNA
gcactgttgaagtgccttaca

SEQ ID NO: 40 - RNA, 21 nt, synthetic construct
  source 1..21: mol_type = other RNA
cacaacgaaatctatgacaag

SEQ ID NO: 41 - RNA, 21 nt, synthetic construct
  source 1..21: mol_type = other RNA
gacctcagctttccctcgagg

SEQ ID NO: 42 - RNA, 21 nt, synthetic construct
  source 1..21: mol_type = other RNA
gaggactgcggatctctgtgt

SEQ ID NO: 43 - RNA, 21 nt, synthetic construct
  source 1..21: mol_type = other RNA
gagtggttatcttttgatgtc

SEQ ID NO: 44 - RNA, 21 nt, synthetic construct
  source 1..21: mol_type = other RNA
ggaacactactgtagttagat

SEQ ID NO: 45 - RNA, 21 nt, synthetic construct
  source 1..21: mol_type = other RNA
gatcgtgcgctcctgcaagtg

SEQ ID NO: 46 - RNA, 21 nt, synthetic construct
  source 1..21: mol_type = other RNA
gcaccttgggcactgttgaag

SEQ ID NO: 47 - RNA, 21 nt, synthetic construct
  source 1..21: mol_type = other RNA
gcttcagctccacggagaaga

SEQ ID NO: 48 - RNA, 21 nt, synthetic construct
  source 1..21: mol_type = other RNA
gcagctgtacattgacttccg

SEQ ID NO: 49 - RNA, 21 nt, synthetic construct
  source 1..21: mol_type = other RNA
gttcttcaacacatcagagct

SEQ ID NO: 50 - RNA, 21 nt, synthetic construct
  source 1..21: mol_type = other RNA
ctcctccaggagacggatctc

SEQ ID NO: 51 - RNA, 21 nt, synthetic construct
  source 1..21: mol_type = other RNA
ccgcgtgctaatggtggaaac

SEQ ID NO: 52 - RNA, 21 nt, synthetic construct
  source 1..21: mol_type = other RNA
cgcatcctagaccctttctcc

SEQ ID NO: 53 - RNA, 21 nt, synthetic construct
  source 1..21: mol_type = other RNA
ctggccaccattcatggcatg

SEQ ID NO: 54 - RNA, 21 nt, synthetic construct
  source 1..21: mol_type = other RNA
ctccaggagacggatctctct

SEQ ID NO: 55 - RNA, 21 nt, synthetic construct
  source 1..21: mol_type = other RNA
ccacagatcccctattcaaga

SEQ ID NO: 56 - RNA, 21 nt, synthetic construct
  source 1..21: mol_type = other RNA
ccaggagacggatctctctcc

SEQ ID NO: 57 - RNA, 21 nt, synthetic construct
  source 1..21: mol_type = other RNA
cctggcgatacctcagcaacc

SEQ ID NO: 58 - RNA, 21 nt, synthetic construct
  source 1..21: mol_type = other RNA
ggctgtatttaaggacacccg

SEQ ID NO: 59 - RNA, 21 nt, synthetic construct
  source 1..21: mol_type = other RNA
ggtgaggaaacaagcccagag

SEQ ID NO: 60 - RNA, 21 nt, synthetic construct
  source 1..21: mol_type = other RNA
ggatagtgaagaagacaataa

SEQ ID NO: 61 - RNA, 21 nt, synthetic construct
  source 1..21: mol_type = other RNA
ggagagaggactgcggatctc

SEQ ID NO: 62 - RNA, 21 nt, synthetic construct
  source 1..21: mol_type = other RNA
ggactatccacctgcaagact

SEQ ID NO: 63 - RNA, 21 nt, synthetic construct
  source 1..21: mol_type = other RNA
gagagaggactgcggatctct

SEQ ID NO: 64 - RNA, 21 nt, synthetic construct
  source 1..21: mol_type = other RNA
gatgcagggactctgataaca

SEQ ID NO: 65 - RNA, 21 nt, synthetic construct
  source 1..21: mol_type = other RNA
gactatccacctgcaagacta

SEQ ID NO: 66 - RNA, 21 nt, synthetic construct
  source 1..21: mol_type = other RNA
gacacgcagtacagcaaggtc

SEQ ID NO: 67 - RNA, 21 nt, synthetic construct
  source 1..21: mol_type = other RNA
ggggccccattaaagatggag

SEQ ID NO: 68 - RNA, 21 nt, synthetic construct
  source 1..21: mol_type = other RNA
gtgacagcagggataacacac

SEQ ID NO: 69 - RNA, 21 nt, synthetic construct
  source 1..21: mol_type = other RNA
gcagggactctgataacaccc

SEQ ID NO: 70 - RNA, 21 nt, synthetic construct
  source 1..21: mol_type = other RNA
cgcgtgctaatggtggaaacc

SEQ ID NO: 71 - RNA, 21 nt, synthetic construct
  source 1..21: mol_type = other RNA
gtggatccacgagcccaaggg

SEQ ID NO: 72 - RNA, 21 nt, synthetic construct
  source 1..21: mol_type = other RNA
gcgtgctaatggtggaaaccc

SEQ ID NO: 73 - RNA, 21 nt, synthetic construct
  source 1..21: mol_type = other RNA
ctgataacacccattttaaag

SEQ ID NO: 74 - RNA, 21 nt, synthetic construct
  source 1..21: mol_type = other RNA
aacccacaacgaaatctatga

SEQ ID NO: 75 - RNA, 21 nt, synthetic construct
  source 1..21: mol_type = other RNA
ctactgtagttagatctattt

SEQ ID NO: 76 - RNA, 21 nt, synthetic construct
  source 1..21: mol_type = other RNA
cctgaacccgtgttgctctcc

SEQ ID NO: 77 - RNA, 21 nt, synthetic construct
  source 1..21: mol_type = other RNA
caccatagcaacactctgaga

SEQ ID NO: 78 - RNA, 21 nt, synthetic construct
  source 1..21: mol_type = other RNA
cacccaccttctggtaccaga

SEQ ID NO: 79 - RNA, 21 nt, synthetic construct
  source 1..21: mol_type = other RNA
cactgcaagtggacatcaacg

SEQ ID NO: 80 - RNA, 21 nt, synthetic construct
  source 1..21: mol_type = other RNA
gcactattcctttgcccggca

SEQ ID NO: 81 - RNA, 21 nt, synthetic construct
  source 1..21: mol_type = other RNA
cgagaagcggtacctgaaccc

SEQ ID NO: 82 - RNA, 21 nt, synthetic construct
  source 1..21: mol_type = other RNA
tggccaccattcatggcatga

SEQ ID NO: 83 - RNA, 21 nt, synthetic construct
  source 1..21: mol_type = other RNA
tacctgaacccgtgttgctct

SEQ ID NO: 84 - RNA, 21 nt, synthetic construct
  source 1..21: mol_type = other RNA
cctcccgcaaagacttttccc

SEQ ID NO: 85 - RNA, 21 nt, synthetic construct
  source 1..21: mol_type = other RNA
ctgttgaagtgccttacatta

SEQ ID NO: 86 - RNA, 21 nt, synthetic construct
  source 1..21: mol_type = other RNA
caccattcatggcatgaaccg

SEQ ID NO: 87 - RNA, 21 nt, synthetic construct
  source 1..21: mol_type = other RNA
gtacattgacttccgcaagga

SEQ ID NO: 88 - RNA, 21 nt, synthetic construct
  source 1..21: mol_type = other RNA
tgacagcagggataacacact

SEQ ID NO: 89 - RNA, 21 nt, synthetic construct
  source 1..21: mol_type = other RNA
cctggacaccaactattgctt

SEQ ID NO: 90 - RNA, 21 nt, synthetic construct
  source 1..21: mol_type = other RNA
gacttccgcaaggacctcggc

SEQ ID NO: 91 - RNA, 21 nt, synthetic construct
  source 1..21: mol_type = other RNA
cacgtggagctgtaccagaaa

SEQ ID NO: 92 - RNA, 21 nt, synthetic construct
  source 1..21: mol_type = other RNA
ccagaaatacagcaacaattc

SEQ ID NO: 93 - RNA, 21 nt, synthetic construct
  source 1..21: mol_type = other RNA
cgcagtacagcaaggtcctgg

SEQ ID NO: 94 - RNA, 21 nt, synthetic construct
  source 1..21: mol_type = other RNA
ccatctaggttatttccgtgg

SEQ ID NO: 95 - RNA, 21 nt, synthetic construct
  source 1..21: mol_type = other RNA
cagagtggttatcttttgatg

SEQ ID NO: 96 - RNA, 21 nt, synthetic construct
  source 1..21: mol_type = other RNA
cctaccttttgccgggagacc

SEQ ID NO: 97 - RNA, 21 nt, synthetic construct
  source 1..21: mol_type = other RNA
cagcatatatatgttcttcaa

SEQ ID NO: 98 - RNA, 21 nt, synthetic construct
  source 1..21: mol_type = other RNA
gcggatctctgtgtcattggg

SEQ ID NO: 99 - RNA, 21 nt, synthetic construct
  source 1..21: mol_type = other RNA
cccctacatttggagcctgga

SEQ ID NO: 100 - RNA, 21 nt, synthetic construct
  source 1..21: mol_type = other RNA
cccacaacgaaatctatgaca

SEQ ID NO: 101 - RNA, 21 nt, synthetic construct
  source 1..21: mol_type = other RNA
gatgtccacttgcagtgtgtt

SEQ ID NO: 102 - RNA, 21 nt, synthetic construct
  source 1..21: mol_type = other RNA
ttctcggagctctgatgtgtt

SEQ ID NO: 103 - RNA, 21 nt, synthetic construct
  source 1..21: mol_type = other RNA
agatctaactacagtagtgtt

SEQ ID NO: 104 - RNA, 21 nt, synthetic construct
  source 1..21: mol_type = other RNA
aggaatagtgcagacaggcag

SEQ ID NO: 105 - RNA, 21 nt, synthetic construct
  source 1..21: mol_type = other RNA
tcggagagagatccgtctcct

SEQ ID NO: 106 - RNA, 21 nt, synthetic construct
  source 1..21: mol_type = other RNA
atgctggttgtacagggccag

SEQ ID NO: 107 - RNA, 21 nt, synthetic construct
  source 1..21: mol_type = other RNA
tctaactacagtagtgttccc

SEQ ID NO: 108 - RNA, 21 nt, synthetic construct
  source 1..21: mol_type = other RNA
ggaaataacctagatgggcgc

SEQ ID NO: 109 - RNA, 21 nt, synthetic construct
  source 1..21: mol_type = other RNA
gaagcaatagttggtgtccag

SEQ ID NO: 110 - RNA, 21 nt, synthetic construct
  source 1..21: mol_type = other RNA
acacgggttcaggtaccgctt

SEQ ID NO: 111 - RNA, 21 nt, synthetic construct
  source 1..21: mol_type = other RNA
aggctccaaatgtaggggcag

SEQ ID NO: 112 - RNA, 21 nt, synthetic construct
  source 1..21: mol_type = other RNA
tccatgtcgatagtcttgcag

SEQ ID NO: 113 - RNA, 21 nt, synthetic construct
  source 1..21: mol_type = other RNA
ataaccactctggcgagtcgc

SEQ ID NO: 114 - RNA, 21 nt, synthetic construct
  source 1..21: mol_type = other RNA
tgaataggggatctgtggcag

SEQ ID NO: 115 - RNA, 21 nt, synthetic construct
  source 1..21: mol_type = other RNA
ttcgttgtgggtttccaccat

SEQ ID NO: 116 - RNA, 21 nt, synthetic construct
  source 1..21: mol_type = other RNA
tcaatttcccctccacggctc

SEQ ID NO: 117 - RNA, 21 nt, synthetic construct
  source 1..21: mol_type = other RNA
tctccatctttaatggggccc

SEQ ID NO: 118 - RNA, 21 nt, synthetic construct
  source 1..21: mol_type = other RNA
ctacagtagtgttccccactg

SEQ ID NO: 119 - RNA, 21 nt, synthetic construct
  source 1..21: mol_type = other RNA
gatcatgttggacagctgctc

SEQ ID NO: 120 - RNA, 21 nt, synthetic construct
  source 1..21: mol_type = other RNA
tatgctggttgtacagggcca

SEQ ID NO: 121 - RNA, 21 nt, synthetic construct
  source 1..21: mol_type = other RNA
agagatccgcagtcctctctc

SEQ ID NO: 122 - RNA, 21 nt, synthetic construct
  source 1..21: mol_type = other RNA
taactacagtagtgttcccca

SEQ ID NO: 123 - RNA, 21 nt, synthetic construct
  source 1..21: mol_type = other RNA
tttcgttgtgggtttccacca

SEQ ID NO: 124 - RNA, 21 nt, synthetic construct
  source 1..21: mol_type = other RNA
ctgcatctcagagtgttgcta

SEQ ID NO: 125 - RNA, 21 nt, synthetic construct
  source 1..21: mol_type = other RNA
agtcaatgtacagctgccgca

SEQ ID NO: 126 - RNA, 21 nt, synthetic construct
  source 1..21: mol_type = other RNA
tgaagcaatagttggtgtcca

SEQ ID NO: 127 - RNA, 21 nt, synthetic construct
  source 1..21: mol_type = other RNA
caggaccttgctgtactgcgt

SEQ ID NO: 128 - RNA, 21 nt, synthetic construct
  source 1..21: mol_type = other RNA
cacgatcatgttggacagctg

SEQ ID NO: 129 - RNA, 21 nt, synthetic construct
  source 1..21: mol_type = other RNA
tctcagagtgttgctatggtg

SEQ ID NO: 130 - RNA, 21 nt, synthetic construct
  source 1..21: mol_type = other RNA
tcaaccactgccgcacaactc

SEQ ID NO: 131 - RNA, 21 nt, synthetic construct
  source 1..21: mol_type = other RNA
catgaatggtggccaggtcac

SEQ ID NO: 132 - RNA, 21 nt, synthetic construct
  source 1..21: mol_type = other RNA
tatgctgtgtgtactctgctt

SEQ ID NO: 133 - RNA, 21 nt, synthetic construct
  source 1..21: mol_type = other RNA
gtcaatgtacagctgccgcac

SEQ ID NO: 134 - RNA, 21 nt, synthetic construct
  source 1..21: mol_type = other RNA
gcaaaggaatagtgcagacag

SEQ ID NO: 135 - RNA, 21 nt, synthetic construct
  source 1..21: mol_type = other RNA
gaactcctcccttaacctctc

SEQ ID NO: 136 - RNA, 21 nt, synthetic construct
  source 1..21: mol_type = other RNA
gagaaagggtctaggatgcgc

SEQ ID NO: 137 - RNA, 21 nt, synthetic construct
  source 1..21: mol_type = other RNA
tatcgccaggaattgttgctg

SEQ ID NO: 138 - RNA, 21 nt, synthetic construct
  source 1..21: mol_type = other RNA
tgcagtgtgttatccctgctg

SEQ ID NO: 139 - RNA, 21 nt, synthetic construct
  source 1..21: mol_type = other RNA
taaggcacttcaacagtgccc

SEQ ID NO: 140 - RNA, 21 nt, synthetic construct
  source 1..21: mol_type = other RNA
tgtcatagatttcgttgtggg

SEQ ID NO: 141 - RNA, 21 nt, synthetic construct
  source 1..21: mol_type = other RNA
tcgagggaaagctgaggtcct

SEQ ID NO: 142 - RNA, 21 nt, synthetic construct
  source 1..21: mol_type = other RNA
acagagatccgcagtcctctc

SEQ ID NO: 143 - RNA, 21 nt, synthetic construct
  source 1..21: mol_type = other RNA
catcaaaagataaccactctg

SEQ ID NO: 144 - RNA, 21 nt, synthetic construct
  source 1..21: mol_type = other RNA
ctaactacagtagtgttcccc

SEQ ID NO: 145 - RNA, 21 nt, synthetic construct
  source 1..21: mol_type = other RNA
cttgcaggagcgcacgatcat

SEQ ID NO: 146 - RNA, 21 nt, synthetic construct
  source 1..21: mol_type = other RNA
tcaacagtgcccaaggtgctc

SEQ ID NO: 147 - RNA, 21 nt, synthetic construct
  source 1..21: mol_type = other RNA
ttctccgtggagctgaagcaa

SEQ ID NO: 148 - RNA, 21 nt, synthetic construct
  source 1..21: mol_type = other RNA
gaagtcaatgtacagctgccg

SEQ ID NO: 149 - RNA, 21 nt, synthetic construct
  source 1..21: mol_type = other RNA
ctctgatgtgttgaagaacat

SEQ ID NO: 150 - RNA, 21 nt, synthetic construct
  source 1..21: mol_type = other RNA
gatccgtctcctggaggagaa

SEQ ID NO: 151 - RNA, 21 nt, synthetic construct
  source 1..21: mol_type = other RNA
ttccaccattagcacgcgggt

SEQ ID NO: 152 - RNA, 21 nt, synthetic construct
  source 1..21: mol_type = other RNA
agaaagggtctaggatgcgcg

SEQ ID NO: 153 - RNA, 21 nt, synthetic construct
  source 1..21: mol_type = other RNA
tgccatgaatggtggccaggt

SEQ ID NO: 154 - RNA, 21 nt, synthetic construct
  source 1..21: mol_type = other RNA
agagatccgtctcctggagga

SEQ ID NO: 155 - RNA, 21 nt, synthetic construct
  source 1..21: mol_type = other RNA
ttgaataggggatctgtggca

SEQ ID NO: 156 - RNA, 21 nt, synthetic construct
  source 1..21: mol_type = other RNA
agagagatccgtctcctggag

SEQ ID NO: 157 - RNA, 21 nt, synthetic construct
  source 1..21: mol_type = other RNA
ttgctgaggtatcgccaggaa

SEQ ID NO: 158 - RNA, 21 nt, synthetic construct
  source 1..21: mol_type = other RNA
ggtgtccttaaatacagcccc

SEQ ID NO: 159 - RNA, 21 nt, synthetic construct
  source 1..21: mol_type = other RNA
ctgggcttgtttcctcacctt

SEQ ID NO: 160 - RNA, 21 nt, synthetic construct
  source 1..21: mol_type = other RNA
attgtcttcttcactatcccc

SEQ ID NO: 161 - RNA, 21 nt, synthetic construct
  source 1..21: mol_type = other RNA
gatccgcagtcctctctccat

SEQ ID NO: 162 - RNA, 21 nt, synthetic construct
  source 1..21: mol_type = other RNA
tcttgcaggtggatagtcccg

SEQ ID NO: 163 - RNA, 21 nt, synthetic construct
  source 1..21: mol_type = other RNA
agatccgcagtcctctctcca

SEQ ID NO: 164 - RNA, 21 nt, unidentified
  source 1..21: mol_type = other RNA
ttatcagagtccctgcatctc

SEQ ID NO: 165 - RNA, 21 nt, synthetic construct
  source 1..21: mol_type = other RNA
gtcttgcaggtggatagtccc

SEQ ID NO: 166 - RNA, 21 nt, synthetic construct
  source 1..21: mol_type = other RNA
ccttgctgtactgcgtgtcca

SEQ ID NO: 167 - RNA, 21 nt, synthetic construct
  source 1..21: mol_type = other RNA
ccatctttaatggggccccag

SEQ ID NO: 168 - RNA, 21 nt, synthetic construct
  source 1..21: mol_type = other RNA
gtgttatccctgctgtcacag

SEQ ID NO: 169 - RNA, 21 nt, synthetic construct
  source 1..21: mol_type = other RNA
gtgttatcagagtccctgcat

SEQ ID NO: 170 - RNA, 21 nt, synthetic construct
  source 1..21: mol_type = other RNA
tttccaccattagcacgcggg

SEQ ID NO: 171 - RNA, 21 nt, synthetic construct
  source 1..21: mol_type = other RNA
cttgggctcgtggatccactt

SEQ ID NO: 172 - RNA, 21 nt, synthetic construct
  source 1..21: mol_type = other RNA
gtttccaccattagcacgcgg

SEQ ID NO: 173 - RNA, 21 nt, synthetic construct
  source 1..21: mol_type = other RNA
ttaaaatgggtgttatcagag

SEQ ID NO: 174 - RNA, 21 nt, synthetic construct
  source 1..21: mol_type = other RNA
atagatttcgttgtgggtttc

SEQ ID NO: 175 - RNA, 21 nt, synthetic construct
  source 1..21: mol_type = other RNA
atagatctaactacagtagtg

SEQ ID NO: 176 - RNA, 21 nt, synthetic construct
  source 1..21: mol_type = other RNA
agagcaacacgggttcaggta

SEQ ID NO: 177 - RNA, 21 nt, synthetic construct
  source 1..21: mol_type = other RNA
tcagagtgttgctatggtgac

SEQ ID NO: 178 - RNA, 21 nt, synthetic construct
  source 1..21: mol_type = other RNA
tggtaccagaaggtgggtggt

SEQ ID NO: 179 - RNA, 21 nt, synthetic construct
  source 1..21: mol_type = other RNA
ttgatgtccacttgcagtgtg

SEQ ID NO: 180 - RNA, 21 nt, synthetic construct
  source 1..21: mol_type = other RNA
ccgggcaaaggaatagtgcag

SEQ ID NO: 181 - RNA, 21 nt, synthetic construct
  source 1..21: mol_type = other RNA
gttcaggtaccgcttctcgga

SEQ ID NO: 182 - RNA, 21 nt, synthetic construct
  source 1..21: mol_type = other RNA
atgccatgaatggtggccagg

SEQ ID NO: 183 - RNA, 21 nt, synthetic construct
  source 1..21: mol_type = other RNA
agcaacacgggttcaggtacc

SEQ ID NO: 184 - RNA, 21 nt, synthetic construct
  source 1..21: mol_type = other RNA
gaaaagtctttgcgggaggcc

SEQ ID NO: 185 - RNA, 21 nt, synthetic construct
  source 1..21: mol_type = other RNA
atgtaaggcacttcaacagtg

SEQ ID NO: 186 - RNA, 21 nt, synthetic construct
  source 1..21: mol_type = other RNA
gttcatgccatgaatggtggc

SEQ ID NO: 187 - RNA, 21 nt, synthetic construct
  source 1..21: mol_type = other RNA
cttgcggaagtcaatgtacag

SEQ ID NO: 188 - RNA, 21 nt, synthetic construct
  source 1..21: mol_type = other RNA
tgtgttatccctgctgtcaca

SEQ ID NO: 189 - RNA, 21 nt, synthetic construct
  source 1..21: mol_type = other RNA
gcaatagttggtgtccagggc

SEQ ID NO: 190 - RNA, 21 nt, synthetic construct
  source 1..21: mol_type = other RNA
cgaggtccttgcggaagtcaa

SEQ ID NO: 191 - RNA, 21 nt, synthetic construct
  source 1..21: mol_type = other RNA
tctggtacagctccacgtgct

SEQ ID NO: 192 - RNA, 21 nt, synthetic construct
  source 1..21: mol_type = other RNA
attgttgctgtatttctggta

SEQ ID NO: 193 - RNA, 21 nt, synthetic construct
  source 1..21: mol_type = other RNA
aggaccttgctgtactgcgtg

SEQ ID NO: 194 - RNA, 21 nt, synthetic construct
  source 1..21: mol_type = other RNA
acggaaataacctagatgggc

SEQ ID NO: 195 - RNA, 21 nt, synthetic construct
  source 1..21: mol_type = other RNA
tcaaaagataaccactctggc

SEQ ID NO: 196 - RNA, 21 nt, synthetic construct
  source 1..21: mol_type = other RNA
tctcccggcaaaaggtaggag

SEQ ID NO: 197 - RNA, 21 nt, synthetic construct
  source 1..21: mol_type = other RNA
gaagaacatatatatgctgtg

SEQ ID NO: 198 - RNA, 21 nt, synthetic construct
  source 1..21: mol_type = other RNA
caatgacacagagatccgcag

SEQ ID NO: 199 - RNA, 21 nt, synthetic construct
  source 1..21: mol_type = other RNA
caggctccaaatgtaggggca

SEQ ID NO: 200 - RNA, 21 nt, synthetic construct
  source 1..21: mol_type = other RNA
tcatagatttcgttgtgggtt

SEQ ID NO: 201-234 - skipped (34 entries)

SEQ ID NO: 235 - RNA, 23 nt, synthetic construct
  source 1..23: mol_type = other RNA
agggcgcgaagggcggcggcggc

SEQ ID NO: 236 - RNA, 17 nt, synthetic construct
  source 1..17: mol_type = other RNA
agggcggcatgggggag

SEQ ID NO: 237 - RNA, 17 nt, synthetic construct
  source 1..17: mol_type = other RNA
gagggcggcatggggga

SEQ ID NO: 238 - RNA, 18 nt, synthetic construct
  source 1..18: mol_type = other RNA
agggcggcatgggggagg

SEQ ID NO: 239 - RNA, 18 nt, synthetic construct
  source 1..18: mol_type = other RNA
gagggcggcatgggggag

SEQ ID NO: 240 - RNA, 18 nt, synthetic construct
  source 1..18: mol_type = other RNA
ggagggcggcatggggga

SEQ ID NO: 241 - RNA, 19 nt, synthetic construct
  source 1..19: mol_type = other RNA
agggcggcatgggggaggc

SEQ ID NO: 242 - RNA, 19 nt, synthetic construct
  source 1..19: mol_type = other RNA
gagggcggcatgggggagg

SEQ ID NO: 243 - RNA, 19 nt, synthetic construct
  source 1..19: mol_type = other RNA
ggagggcggcatgggggag

SEQ ID NO: 244 - RNA, 19 nt, synthetic construct
  source 1..19: mol_type = other RNA
cggagggcggcatggggga

SEQ ID NO: 245 - RNA, 20 nt, synthetic construct
  source 1..20: mol_type = other RNA
agggcggcatgggggaggcg

SEQ ID NO: 246 - RNA, 20 nt, synthetic construct
  source 1..20: mol_type = other RNA
gagggcggcatgggggaggc

SEQ ID NO: 247 - RNA, 20 nt, synthetic construct
  source 1..20: mol_type = other RNA
ggagggcggcatgggggagg

SEQ ID NO: 248 - RNA, 20 nt, synthetic construct
  source 1..20: mol_type = other RNA
cggagggcggcatgggggag

SEQ ID NO: 249 - RNA, 20 nt, synthetic construct
  source 1..20: mol_type = other RNA
ccggagggcggcatggggga

SEQ ID NO: 250 - RNA, 21 nt, synthetic construct
  source 1..21: mol_type = other RNA
agggcggcatgggggaggcgg

SEQ ID NO: 251 - RNA, 21 nt, synthetic construct
  source 1..21: mol_type = other RNA
gagggcggcatgggggaggcg

SEQ ID NO: 252 - RNA, 21 nt, synthetic construct
  source 1..21: mol_type = other RNA
ggagggcggcatgggggaggc

SEQ ID NO: 253 - RNA, 21 nt, synthetic construct
  source 1..21: mol_type = other RNA
cggagggcggcatgggggagg

SEQ ID NO: 254 - RNA, 21 nt, synthetic construct
  source 1..21: mol_type = other RNA
ccggagggcggcatgggggag

SEQ ID NO: 255 - RNA, 21 nt, synthetic construct
  source 1..21: mol_type = other RNA
cccggagggcggcatggggga

SEQ ID NO: 256 - RNA, 22 nt, synthetic construct
  source 1..22: mol_type = other RNA
agggcggcatgggggaggcggc

SEQ ID NO: 257 - RNA, 23 nt, synthetic construct
  source 1..23: mol_type = other RNA
cccggagggcggcatgggggagg

SEQ ID NO: 258 - RNA, 18 nt, synthetic construct
  source 1..18: mol_type = other RNA
gagggcggcatgggggag

SEQ ID NO: 259 - RNA, 19 nt, synthetic construct
  source 1..19: mol_type = other RNA
gagggcggcatgggggagg

SEQ ID NO: 260 - RNA, 20 nt, synthetic construct
  source 1..20: mol_type = other RNA
agggcggcatgggggaggcg

SEQ ID NO: 261 - RNA, 20 nt, synthetic construct
  source 1..20: mol_type = other RNA
gagggcggcatgggggaggc

SEQ ID NO: 262 - RNA, 20 nt, synthetic construct
  source 1..20: mol_type = other RNA
ggagggcggcatgggggagg

SEQ ID NO: 263 - RNA, 20 nt, synthetic construct
  source 1..20: mol_type = other RNA
cggagggcggcatgggggag

SEQ ID NO: 264 - protein, 446 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 265 - protein, 214 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC